(19) United States
(12) Patent Application Publication
(10) Pub. No.: US 2024/0360185 A1
(43) Pub. Date: Oct. 31, 2024

(54) AV3 MUTANT INSECTICIDAL POLYPEPTIDES AND METHODS FOR PRODUCING AND USING SAME

(71) Applicant: Vestaron Corporation, Durham, NC (US)

(72) Inventors: Robert M. KENNEDY, Dexter, MI (US); Lin BAO, Portage, MI (US)

(21) Appl. No.: 18/642,377

(22) Filed: Apr. 22, 2024

Related U.S. Application Data
(62) Division of application No. 17/276,050, filed on Mar. 12, 2021, now abandoned.
(60) Provisional application No. 62/731,387, filed on Sep. 14, 2018.

(51) Int. Cl. C07K 14/435; A01N 63/50; C12N 15/82

(52) U.S. Cl. CPC C07K 14/43504; A01N 63/50; C12N 15/8286

(57) ABSTRACT

New insecticidal proteins, nucleotides, peptides, their expression in plants, methods of producing the peptides, new processes, production techniques, new peptides, new formulations, and new organisms, a process which increases the insecticidal peptide production yield from yeast expression systems. The present disclosure is also related and discloses toxins called AVPs, which are modified from the Av3 toxin derived from sea anemone; here we describe the genes encoding the new polypeptide, as well various formulations and combinations; of both genes and peptides, useful for the control of insects.

### CROSS REFERENCE TO RELATED APPLICATIONS

This application is a divisional application of U.S. patent application Ser. No. 17/276,050, filed on Mar. 12, 2021, which is a 35 U.S.C. § 371 United States National Phase Application of PCT Application No. PCT/US2019/051093, filed on Sep. 13, 2019, which claims the benefit of, and priority to, U.S. Provisional Application Ser. No. 62/731,387, filed on Sep. 14, 2018, the disclosures of which are incorporated by reference herein in their entireties.

### SEQUENCE

This application incorporates by reference in its entirety the Sequence Listing XML entitled “277702-545673.xml” (79,493 bytes), which was created on Apr. 18, 2024, and filed electronically-herewith.

### TECHNICAL FIELD

New insecticidal proteins, nucleotides, peptides, their expression in plants, methods of producing the peptides, new processes, production techniques, new peptides, new formulations, and combinations of new and known organisms that produce greater yields than would be expected of related peptides for the control of insects are described and claimed.

### BACKGROUND

Arthropod vectors, for example, mosquitoes, flea, ticks, lice, and flies can transmit diseases to human and animals, and create great public health concern, However, most current vector insecticides have been in existence for over 40 years. Some of them have been banned or restricted by a regulatory agency. Several insect vectors have developed resistance to many classes of insecticides, including Bacillus thuringiensis (Bt) protein crystals, pyrethrins, etc. (See for example, Brogdon W G, McAllister J C, Emerg Infect Dis 1998, 4(4):605-613; Rose RI: Emerg. Infect. Dis. 2001, 7(1):17-23).

Numerous insects are vectors for disease. Mosquitoes in the genus Anopheles are the principle vectors of Zika virus, Chikungunya virus, and malaria, a disease caused by protozoa in the genus Trypanosoma. Aedes aegypti is the main vector of the viruses that cause Yellow fever and Dengue. Other viruses, the causal agents of various types of encephalitis, are also carried by Aedes spp. mosquitoes. Wuchereria bancrofti and Brugia malayi, parasitic roundworms that cause filariasis, are usually spread by mosquitoes in the genera Culex, Mansonia, and Anopheles.

Horse flies and deer flies may transmit the bacterial pathogens of tularemia (Pasteurella tularensis) and anthrax (Bacillus anthracis), as well as a parasitic roundworm (Loa loa) that causes loiasis in tropical Africa.

Eye gnats in the genus Hippelates can carry the spirochaete pathogen that causes yaws (Treponema pertenue), and may also spread conjunctivitis (pinkeye). Tsetse flies in the genus Glossina transmit the protozoan pathogens that cause African sleeping sickness (Trypanosoma gambiense and T. rhodesiense). Sand flies in the genus Phlebotomus are vectors of a bacterium (Bartonella bacilliformis) that causes Carrion's disease (oroyo fever) in South America. In parts of Asia and North Africa, they spread a viral agent that causes sand fly fever (pappataci fever) as well as protozoan pathogens (Leishmania spp.) that cause Leishmaniasis.

The global security of food produced by modern agriculture and horticulture is challenged by insect pests. Farmers rely on insecticides to suppress insect damage, yet commercial options for safe and functional insecticides available to farmers are diminishing through the removal of dangerous chemicals from the marketplace and the evolution of insect strains that are resistant to all major classes of chemical and biological insecticides. Accordingly, new insecticides are necessary for farmers to maintain crop protection.

Insecticidal polypeptides are polypeptides that are toxic to their targets, usually pests (e.g., insects or arachnids of some type). Such polypeptides may be delivered internally, for example by delivering the toxin directly to the insect's gut or internal organs by injection or by inducing the insect to consume the toxin from its food, for example an insect feeding upon a transgenic plant, and/or they may have the ability to inhibit the growth, impair the movement, or even kill an insect when the toxin is delivered to the insect by spreading the toxin to locus inhabited by the insect or to the insect's environment by spraying, or other means, and then the insect comes into some form of contact with the polypeptide.

Insecticidal polypeptides however have enormous problems reaching the commercial market, and to date there have been few if any insecticidal polypeptides approved and marketed for the commercial market (i.e., with the exception of peptides derived from Bacillis thuringiensis or Bt).

A polypeptide that has shown some promise as being an insecticidal polypeptide is an Av3 toxin from the sea anemone, Anemonia viridis. Av3 is a type III sea anemone toxin that inhibits the inactivation of voltage-gated sodium (Na⁺) channels at receptor site 3, resulting in contractile paralysis. (Blumenthal et al., Voltage-gated sodium channel toxins: poisons, probes, and future promise. Cell Biochem Biophys. 2003; 38(2):215-38). While Av3 shows to exhibit its effect on insect sodium channels, it does not show selectivity for mammalian sodium channels (Moran et al., Sea anemone toxins affecting voltage-gated sodium channels—molecular and evolutionary features, Toxicon. 2009 Dec. 15; 54(8): 1089-1101).

The ability to successfully produce insecticidal peptides on a commercial scale, with reproducible peptide formation and folding, at a reasonable and economical price, can be challenging. The wide variety, unique properties and special nature of insecticidal peptides, combined with the huge variety of possible production techniques, can present an overwhelming number of approaches to peptide application and production, but few, if any, are commercially successful.

There are several reasons why so few of the multitude insecticidal polypeptides that have been identified have ever made it to market. First, most insecticidal polypeptides are either too delicate, and/or not toxic enough to be commercially successful. Second, insecticidal polypeptides are difficult and costly to produce, rendering them economically unviable. Third, many insecticidal polypeptides degrade quickly, and have a short half-life. Fourth, very few insecticidal polypeptides fold properly when then are expressed by a plant, thus they lose their toxicity in genetically modified organisms (GMOs). Fifth, most of the identified insecticidal polypeptides are blocked from systemic distribution in the insect and/or lose their toxic nature when consumed by insects.

There is a need to provide solutions to these problems.

### SUMMARY

The present disclosure provides an Av3 variant polypeptide (AVP), compositions comprising an AVP, insecticidal proteins comprising one or more AVPs optionally with other proteins, and methods for their use to eradicate, kill, control, inhibit, injure, render sterile or combinations thereof, one or more insect species. The AVPs described herein have insecticidal activity against one or more insect species. AVPs of the present disclosure have at least one of the following mutations: (1) an N-terminal mutation replacing the amino terminal Arginine with Lysine (R1K) amino acid relative to SEQ ID NO:1; (2) a deletion of the C-terminal valine amino acid relative to SEQ ID NO: 1. The AVP described herein have been shown to have a knockdown of 50% of the population concentration (KD₅₀) of lower than 100 ppm against mosquitos at 3-hours post application.

In addition, the present disclosure provides for an AVP that further comprises a homopolymer or heteropolymer of two or more AVP polypeptides, wherein the amino acid sequence of each AVP is the same or different; an AVP comprising a fused protein comprising two or more AVP polypeptides separated by a cleavable or non-cleavable linker, and wherein the amino acid sequence of each AVP may be the same or different, wherein the linker is cleavable inside the gut or hemolymph of an insect; and compositions comprising one or more of the foregoing AVPs, and combinations thereof, and an excipient.

In addition, the present disclosure provides a composition comprising an AVP, and/or an insecticidal protein comprising one or more AVPs, and an excipient; a plant, plant tissue, plant cell, or plant seed comprising an AVP, and/or an insecticidal protein comprising one or more AVPs, or a polynucleotide encoding one or more AVPs, wherein the AVP described in the foregoing compositions, plant, plant cell, plant seed or methods, comprises at least one mutation selected from: an N-terminal mutation replacing the amino terminal arginine (R) amino acid with a lysine (K) amino acid (R1K) relative to SEQ ID NO:1; and a deletion of the C-terminal valine (v) amino acid, relative to SEQ ID NO:1. In some embodiments, an exemplary composition, insecticidal protein, plant, plant cell, or plant seed of any of the foregoing, comprise at least one AVP polypeptide or a polynucleotide encoding an AVP or a complementary nucleic acid operable to hybridize with a polynucleotide operable to encode an AVP, or insecticidal protein comprising at least one AVP, wherein the AVP, in any of the forgoing examples of compositions, insecticidal protein, plant, plant cell, plant seed or methods of making and methods of using thereof, comprises both mutations, i.e. an N-terminal mutation replacing the amino terminal arginine (R) amino acid with a lysine (K) amino acid (R1K) relative to SEQ ID NO:1, and a deletion of the C-terminal valine (v) amino acid, relative to SEQ ID NO:1.

In addition, the present disclosure provides a polynucleotide operable to encode an AVP, wherein the AVP comprises at least one mutation selected from an N-terminal mutation replacing the amino terminal arginine (R) amino acid with a lysine (K) amino acid (R1K) relative to SEQ ID NO: 1; and a deletion of the C-terminal valine (v) amino acid, relative to SEQ ID NO:1. Also, the present disclosure provides a polynucleotide wherein the polynucleotide encodes a AVP having an N-terminal mutation replacing the amino terminal arginine (R) amino acid with a lysine (K) amino acid (R1K) relative to SEQ ID NO:1, and a deletion of the C-terminal valine (v) amino acid, relative to SEQ ID NO:1.

The present disclosure provides a method of producing an AVP, the method comprising: preparing a vector comprising a SSI expression cassette comprising a polynucleotide operable to express a mutant Av3 polypeptide having at least one mutation selected from: an N-terminal mutation and a C-terminal mutation relative to the wild-type sequence of Av3 as set forth in SEQ ID NO:1; introducing the vector into a yeast strain; growing the yeast strain in a growth medium under conditions operable to enable expression of the AVP and secretion into the growth medium, and isolating the expressed AVP from the growth medium.

The present disclosure provides a method of producing an AVP, wherein the N-terminal mutation is an amino acid substitution of R1K relative to SEQ ID NO:1; an AVP wherein C-terminal mutation is an amino acid deletion of the C-terminal valine relative to SEQ ID NO:1; and an AVP wherein the polynucleotide encodes a mutant Av3 polypeptide having an N-terminal mutation comprising an amino acid substitution of R1K relative to SEQ ID NO:1, and a C-terminal mutation comprising an amino acid deletion of the C-terminal valine relative to SEQ ID NO:1.

In some embodiments of the present disclosure, an illustrative method is provided which utilizes a plasmid comprising an alpha-MF signal; a Kex 2 cleavage site, which is transformed into a yeast strain, such as Kluyveromyces lactis, Saccharomyces cerevisiae, Pichia pastoris and/or other species of yeast.

According to the methods and techniques taught in this disclosure, the expression of the AVP in a yeast culture provides a yield of at least: 70 mg/L, 80 mg/L, 90 mg/L, 100 mg/L, 110 mg/L, 120 mg/L, 130 mg/L, 140 mg/L, 150 mg/L, 160 mg/L, 170 mg/L, 180 mg/L, 190 mg/L 200 mg/L, 500 mg/L, 750 mg/L, 1,000 mg/L, 1,250 mg/L, 1,500 mg/L, 1,750 mg/L or at least 2,000 mg/L of AVP per liter of culture medium.

The present disclosure also provides an AVP with the following amino acid sequences: (1) an AVP comprising the amino acid sequence X₁-X₂-C-C-P-C-Y-W-G-G-C-P-W-G-X₃-X₄-C-Y-P-X₅-G-C-X₆-X₇-X₈-X₉-X₁₀ (SEQ ID NO: 35); wherein X₁ is H, K, D, E, S, T, N, Q, C, G, P, A, V, I, L, M, F, Y, or W; X₂ is R, H, K, D, E, S, T, N, Q, C, G, P, A, V, I, L, M, F, Y, or W; X₃ is R, H, K, D, E, S, T, N, Q, C, G, P, A, V, I, L, M, F, Y, or W; X₄ is R, H, K, D, E, S, T, N, Q, C, G, P, A, V, I, L, M, F, Y, or W; X₅ is R, H, K, D, E, S, T, N, Q, C, G, P, A, V, I, L, M, F, Y, or W; X₆ is R, H, K, D, E, S, T, N, Q, C, G, P, A, V, I, L, M, F, Y, or W; X₇ is R, H, K, D, E, S, T, N, Q, C, G, P, A, V, I, L, M, F, Y, or W; X₈ is R, H, K, D, E, S, T, N, Q, C, G, P, A, V, I, L, M, F, Y, or W; X₉ is R, H, K, D, E, S, T, N, Q, C, G, P, A, V, I, L, M, F, Y, or W; and X₁₀ is R, H, K, D, E, S, T, N, Q, C, G, P, A, V, I, L, M, F, Y, W or absent; and (2) an AVP comprising the amino acid sequence X₁-X₂-C-C-P-C-Y-W-G-G-C-P-W-G-X₃-X₄-C-Y-P-X₅-G-C-X₆-X₇-X₈-X₉-X₁₀ (SEQ ID NO: 36); wherein X₁ is R, H, K, D, E, S, T, N, Q, C, G, P, A, V, I, L, M, F, Y, or W; X₂ is R, H, K, D, E, S, T, N, Q, C, G, P, A, V, I, L, M, F, Y, or W; X₃ is R, H, K, D, E, S, T, N, Q, C, G, P, A, V, I, L, M, F, Y, or W; X₄ is R, H, K, D, E, S, T, N, Q, C, G, P, A, V, I, L, M, F, Y, or W; X₅ is R, H, K, D, E, S, T, N, Q, C, G, P, A, V, I, L, M, F, Y, or W; X₆ is R, H, K, D, E, S, T, N, Q, C, G, P, A, V, I, L, M, F, Y, or W; X₇ is R, H, K, D, E, S, T, N, Q, C, G, P, A, V, I, L, M, F, Y, or W; X₈ is R, H, K, D, E, S, T, N, Q, C, G, P, A, V, I, L, M, F, Y, or W; X₉ is R, H, K, D, E, S, T, N, Q, C, G, P, A, V, I, L, M, F, Y, or W; and X₁₀ is absent. In some embodiments, the AVP will have an amino acid sequence comprising KACCPCYWGGCPWGAACYPAGCAAAK of SEQ ID NO:30.

In addition, the present disclosure provides a plant, plant tissue, plant cell, or plant seed comprising an AVP or a polynucleotide encoding the same, wherein the AVP comprises an AVP polypeptide, and any combinations thereof. In some embodiments, the plant, plant tissue, plant cell or seed has an AVP that is selected from the group consisting of an AVP with the sequence (1) X₁-X₂-C-C-P-C-Y-W-G-G-C-P-W-G-X₃-X₄-C-Y-P-X₅-G-C-X₆-X₇-X₈-X₉-X₁₀ (SEQ ID NO: 35); wherein X₁ is H, K, D, E, S, T, N, Q, C, G, P, A, V, I, L, M, F, Y, or W; X₂ is R, H, K, D, E, S, T, N, Q, C, G, P, A, V, I, L, M, F, Y, or W; X₃ is R, H, K, D, E, S, T, N, Q, C, G, P, A, V, I, L, M, F, Y, or W; X₄ is R, H, K, D, E, S, T, N, Q, C, G, P, A, V, I, L, M, F, Y, or W; X₈ is R, H, K, D, E, S, T, N, Q, C, G, P, A, V, I, L, M, F, Y, or W; X₆ is R, H, K, D, E, S, T, N, Q, C, G, P, A, V, I, L, M, F, Y, or W; X₇ is R, H, K, D, E, S, T, N, Q, C, G, P, A, V, I, L, M, F, Y, or W; X₈ is R, H, K, D, E, S, T, N, Q, C, G, P, A, V, I, L, M, F, Y, or W; X₉ is R, H, K, D, E, S, T, N, Q, C, G, P, A, V, I, L, M, F, Y, or W; and X₁₀ is R, H, K, D, E, S, T, N, Q, C, G, P, A, V, I, L, M, F, Y, W or absent; and (2) an AVP comprising the amino acid sequence X₁-X₂-C-C-P-C-Y-W-G-G-C-P-W-G-X₃-X₄-C-Y-P-X₅-G-C-X₆-X₇-X₈-X₉-X₁₀ (SEQ ID NO: 36); wherein X₁ is R, H, K, D, E, S, T, N, Q, C, G, P, A, V, I, L, M, F, Y, or W; X₂ is R, H, K, D, E, S, T, N, Q, C, G, P, A, V, I, L, M, F, Y, or W; X₃ is R, H, K, D, E, S, T, N, Q, C, G, P, A, V, I, L, M, F, Y, or W; X₄ is R, H, K, D, E, S, T, N, Q, C, G, P, A, V, I, L, M, F, Y, or W; X₅ is R, H, K, D, E, S, T, N, Q, C, G, P, A, V, I, L, M, F, Y, or W; X₆ is R, H, K, D, E, S, T, N, Q, C, G, P, A, V, I, L, M, F, Y, or W; X₇ is R, H, K, D, E, S, T, N, Q, C, G, P, A, V, I, L, M, F, Y, or W; X₈ is R, H, K, D, E, S, T, N, Q, C, G, P, A, V, I, L, M, F, Y, or W; X₉ is R, H, K, D, E, S, T, N, Q, C, G, P, A, V, I, L, M, F, Y, or W; and X₁₀ is absent; and (3) any combinations thereof; the present disclosure also provides a polynucleotide operable to encode an AVP selected from any one of foregoing AVP sequences.

The present disclosure provides an exemplary method wherein the expression of an illustrative AVP in a culture medium, for example, a yeast fermentation medium fed by provision of a fermentable sugar, e.g. galactose, maltose, latotriose, sucrose, fructose or glucose and/or a sugar alcohol, for example, erythritol, hydrogenated starch hydrolysates, isomalt, lactitol, maltitol, mannitol, and xylitol results in the expression of a single AVP in the medium or at least a single polypeptide in an amount of at least 95%, or at least 96%, or at least 97%, or at least 98%, or at least 99% (w/w) when compared to the quantities of other mutants of Av3 polypeptide having a mutation other than those of AVPs in the amino acid sequence of SEQ ID NO:1 in the yeast culture medium. In one embodiment, the AVP expressed in said yeast culture comprises a single mutation, i.e. an N-terminal mutation replacing the amino terminal arginine (R) amino acid with a lysine (K) amino acid (R1K) relative to SEQ ID NO:1. In another embodiment, the AVP expressed in said yeast culture described above comprises a single mutation, wherein the AVP comprises a deletion of the C-terminal valine (v) amino acid, relative to SEQ ID NO: 1. In still further embodiments, the AVP expressed in said yeast culture described above comprises a dual mutation, namely an N-terminal mutation replacing the amino terminal arginine (R) amino acid with a lysine (K) amino acid (R1K) relative to SEQ ID NO: 1, and a deletion of the C-terminal valine (v) amino acid, relative to SEQ ID NO:1.

In some embodiments, illustrative methods of making an exemplary AVP polypeptide as described in the present disclosure provides a method of making an AVP recombinantly, wherein a suitable expression vector, for example, a yeast expression vector, comprises two or three expression cassettes, each expression cassette operable to encode the AVP of the SSI expression cassette.

### BRIEF DESCRIPTION OF THE DRAWINGS

FIG. 1 depicts an rpHPLC Chromatograph showing expressed Av3+2 peptide.

FIG. 2 depicts a Liquid chromatography/mass spectrometry (LC/MS) Waters/Micromass ESI-TOF mass spectrometer readings of “Peak 1” in FIG. 1.

FIG. 3 depicts a Liquid chromatography/mass spectrometry (LC/MS) Waters/Micromass ESI-TOF mass spectrometer readings of “Peak 2” in FIG. 1.

FIG. 4 depicts a bar graph showing the results of purified Av3+2 peptide incubated in the fermentation beer with untransformed K. lactis to detect C-terminal valine cleavage to form the polypeptide Av3+2−C1.

FIG. 5 depicts a schematic representation of a vector diagram of the Av3 peptide expression vector used to generate native Av3 polypeptide.

FIG. 6 depicts an rpHPLC Chromatograph showing four peaks in the native Av3 fermentation beer.

FIG. 7 depicts a Liquid chromatography/mass spectrometry (LC/MS) Waters/Micromass ESI-TOF mass spectrometer readings showing the three different isoforms present in native Av3 fermentation beer.

FIG. 8 depicts a Liquid chromatography/mass spectrometry (LC/MS) Waters/Micromass ESI-TOF mass spectrometer readings from the fermentation beer of the pLB103a-YCT strain, which produced the two peptides: Av3a and Av3a-C1.

FIG. 9 depicts a Liquid chromatography/mass spectrometry (LC/MS) Waters/Micromass ESI-TOF mass spectrometer readings from the fermentation beer of the pLB103b-YCT-3 strain, which produced the Av3b peptide, which is the only Av3 related peptide found in the fermentation beer by LC/MS.

FIG. 10 depicts a graph displaying results of a housefly injection assay showing knock-down activity at 4-hours post injection.

FIG. 11 depicts a bar graph showing post-topical knock-down effects in a mosquito topical assay.

FIG. 12 depicts a bar graph showing post-topical knock-down effects in a mosquito topical assay using native Av3 and Av3+2.

FIG. 13 depicts a graph displaying a dose response curve showing the effect of R1K and AC-Val, and native Av3 in mosquito topical assay.

FIG. 14 depicts a schematic representation of a vector diagram of the pLB103b-YCT-3 strain from with a single Av3b expression cassette K. lactis vector.

FIG. 15 depicts a schematic representation of a vector diagram of the double Av3b expression cassette K. lactis expression vector.

FIG. 16 depicts a schematic representation of a vector diagram of the triple Av3b expression cassette K. lactis expression vector.

FIG. 17 depicts a graph displaying the results of a housefly injection assay showing knock-down activity at 4 hours post injection with Av3+2 and Av3.

FIG. 18 depicts a graph displaying the results of a housefly injection assay showing knock-down activity at 4 hours post injection with native Av3 and Av3-C1.

FIG. 19 depicts a graph displaying the results of knock down oral toxicity analysis in housefly with 20 PPT Av3+2−C1 and control at 96 hours.

FIG. 20 depicts a graph displaying the results of mortality in oral toxicity analysis in housefly with 20 PPT Av3+2−C1 and control at 96 hours.

FIG. 21 depicts a photograph displaying the toxicity results of Manduca sexta larvae treated with Av3 fermentation beer from native Av3 strains and pLB103-YCT-3 strains.

FIG. 22 depicts a graph displaying the results of mosquito larva oral toxicity bioassay over 24 hours.

FIG. 23 depicts a graph displaying the results of adult mosquito topical toxicity assay with Av3+2 and Av3+2−C1.

FIG. 24 depicts a graph displaying the results of adult mosquito topical toxicity assay with native Av3 and AVP.

FIG. 25 depicts a graph displaying the synergistic effects of 50% AVPs and Bti proteins.

FIG. 26 depicts a graph displaying the synergistic effects of 25% AVPs and Bti proteins.

FIG. 27 depicts a graph displaying the synergistic effects of native Av3 with 500 ppb permethrin in mosquito feeding bioassay.

FIG. 28 depicts a graph displaying the synergistic effects of AVP with 500 ppb permethrin in mosquito feeding bioassay.

FIG. 29 depicts a graph displaying the housefly knock-down assay using Av3+2 peptide and AaIT1.

FIG. 30 depicts a graph displaying the 24 hour housefly knock-down assay using Av3 native polypeptide versus Av3b polypeptide and their resultant ED₅₀ concentrations.

FIG. 31 depicts an illustration of a 3D model of the tertiary structure of an AVP with the hydrophobic Nicotinic acetylcholine binding surface shown as a yellow-colored area.

FIG. 32 depicts a graph showing the Agilent HLPC validation of yeast-expressed Av3b compared to synthetic AVP polypeptides and AVP-Core polypeptides; here, the peak shift of AVP-core proteins indicates the lack of a disulfide bond.

FIG. 33 depicts a graph showing the results of a housefly injection assay using yeast-expressed Av3b and synthetic Av3b and showing KD50 at 3-hours post injection.

FIG. 34 depicts a graph showing KD50 at 3-hours for housefly injection assay when using yeast-expressed Av3b and AVP-Core synthetic polypeptides Core 5 and Core 4.

FIG. 35 depicts a graph showing polypeptide toxicity after 2.5 hours for AVP-Core synthetic polypeptides AVP-Core-1; AVP-Core-2; AVP-Core-3; and Av3b.

FIG. 36 depicts a graph showing polypeptide toxicity after 24 hours for AVP-Core synthetic polypeptides AVP-Core-1; AVP-Core-2; AVP-Core-3; and Av3b.

### DETAILED DESCRIPTION

### Definitions

The term “5′-end” and “3′-end” refers to the directionality, i.e., the end-to-end orientation of a nucleotide polymer (e.g., DNA). The 5′-end of a polynucleotide is the end of the polynucleotide that has the fifth carbon atom of the furanose oriented away from the center of strand; the 3′-end is the end of the polynucleotide is the end of the polynucleotide that has the third carbon atom of the furanose oriented away from the center of the strand. As used as a term of orientation, directions toward the 5′-end are referred to as “upstream”, and directions toward the 3′-end are referred to as “downstream.”

“Agroinfection” means a plant transformation method where DNA is introduced into a plant cell by using Agrobacteria A. tumefAciens or A. rhizogenes.

“ADN1 promoter” refers to the DNA segment comprised of the promoter sequence derived from the Schizosaccharomyces pombe adhesion defective protein 1 gene.

“Alpha-MF signal” or “aMF secretion signal” refers to a protein that directs nascent recombinant polypeptides to the secretory pathway.

“Agriculturally-acceptable carrier” covers all adjuvants, inert components, dispersants, surfactants, tackifiers, binders, etc. that are ordinarily used in pesticide formulation technology; these are well known to those skilled in pesticide formulation.

“Av3” refers to a polypeptide isolated from the sea anemone, Anemonia viridis, which can target receptor site 3 on α-subunit III of voltage-gated sodium channels. One example of an Av3 polypeptide is an Av3 polypeptide having the amino acid sequence of SEQ ID NO:1 (NCBI Accession No. P01535.1).

“Av3 variant polynucleotide” refers to the polynucleotide sequence that encodes any AVP. The term “Av3 variant polynucleotide” when used to describe the Av3 variant polynucleotide sequence contained in an AVP expression ORF, its inclusion in a vector, and/or when describing the polynucleotides encoding an insecticidal protein, is described as “avp” and/or “Avp”.

“AVP expression cassette” or “AVPs expression vector” refers to one or more regulatory elements such as promoters; enhancer elements; mRNA stabilizing polyadenylation signal; an internal ribosome entry site (IRES); introns; post-transcriptional regulatory elements; and a polynucleotide operable to express an AVP. For example, one example of an AVP expression cassette is one or more segments of DNA that contains a polynucleotide segment operable to express an AVP, an ADH1 promoter, a LAC4 terminator, and an alpha-MF secretory signal.

“AVP expression ORF” refers to a nucleotide encoding an AVP, and/or one or more stabilizing proteins, secretory signals, or target directing signals, for example, ERSP or STA, and is defined as the nucleotides in the ORF that has the ability to be translated.

“AVP expression ORF diagram” refers to the composition of one or more AVP expression ORF s, as written out in diagram or equation form. For example, an “AVP expression ORF diagram” can be written out as using acronyms or short-hand references to the DNA segments contained within the expression ORF. Accordingly, in one example, an “AVP expression ORF diagram” may describe the polynucleotide segments encoding the ERSP, LINKER, STA, and AVP, by diagramming in equation form the DNA segments as “ersp” (i.e., the polynucleotide sequence that encodes the ERSP polypeptide); “linker” or “L” (i.e., the polynucleotide sequence that encodes the LINKER polypeptide); “sta” (i.e., the polynucleotide sequence that encodes the STA polypeptide), and “avp” (i.e., the polynucleotide sequence encoding a AVP), respectively. An example of an AVP expression ORF diagram” is “ersp-sta-(linkerᵢ-avpⱼ)_{N},” or “ersp-(avpⱼ-linkerᵢ)_{N}-sta” and/or any combination of the DNA segments thereof.

“AVP” or “Av3 mutant polypeptides” or “mutant Av3 peptides” or “AVP-Core synthetic polypeptide” or “Core” (used here interchangeably) refer to an Av3 polypeptide sequence and/or a polypeptide encoded by a variant Av3 polynucleotide sequence that has been altered to produce a non-naturally occurring polypeptide and/or polynucleotide sequence. For example, in some embodiments, an “AVP” polypeptide comprises at least one mutation selected from: an N-terminal mutation replacing the amino terminal arginine (R) amino acid with a lysine (K) amino acid (R1K) relative to SEQ ID NO:1; and a deletion of the C-terminal valine (v) amino acid, relative to SEQ ID NO:1. In various embodiments, there are three AVPs, the first AVP comprises a polypeptide with an N-terminal mutation replacing the amino terminal arginine (R) amino acid with a lysine (K) amino acid (R1K) relative to SEQ ID NO: 1. The second AVP is a polypeptide with a deletion of the C-terminal valine (v) amino acid, relative to SEQ ID NO:1. The third AVP is a polypeptide with two mutations, an N-terminal mutation replacing the amino terminal arginine (R) amino acid with a lysine (K) amino acid (R1K) relative to SEQ ID NO:1; and a deletion of the C-terminal valine (v) amino acid, relative to SEQ ID NO:1. In yet other embodiments, an AVP can possess an amino acid sequence comprising X₁-X₂-C-C-P-C-Y-W-G-G-C-P-W-G-X₃-X₄-C-Y-P-X₅-G-C-X₆-X₇-X₈-X₉-X₁₀ (SEQ ID NO: 35); wherein X₁ is H, K, D, E, S, T, N, Q, C, G, P, A, V, I, L, M, F, Y, or W; X₂ is R, H, K, D, E, S, T, N, Q, C, G, P, A, V, I, L, M, F, Y, or W; X₃ is R, H, K, D, E, S, T, N, Q, C, G, P, A, V, I, L, M, F, Y, or W; X₄ is R, H, K, D, E, S, T, N, Q, C, G, P, A, V, I, L, M, F, Y, or W; X₅ is R, H, K, D, E, S, T, N, Q, C, G, P, A, V, I, L, M, F, Y, or W; X₆ is R, H, K, D, E, S, T, N, Q, C, G, P, A, V, I, L, M, F, Y, or W; X₇ is R, H, K, D, E, S, T, N, Q, C, G, P, A, V, I, L, M, F, Y, or W; X₈ is R, H, K, D, E, S, T, N, Q, C, G, P, A, V, I, L, M, F, Y, or W; X₉ is R, H, K, D, E, S, T, N, Q, C, G, P, A, V, I, L, M, F, Y, or W; and X₁₀ is R, H, K, D, E, S, T, N, Q, C, G, P, A, V, I, L, M, F, Y, W or absent. In yet other embodiments, an “AVP” may have an amino acid sequence comprising An AVP comprising the amino acid sequence X₁-X₂-C-C-P-C-Y-W-G-G-C-P-W-G-X₃-X₄-C-Y-P-X₅-G-C-X₆-X₇-X₈-X₉-X₁₀ (SEQ ID NO: 36); wherein X₁ is R, H, K, D, E, S, T, N, Q, C, G, P, A, V, I, L, M, F, Y, or W; X₂ is R, H, K, D, E, S, T, N, Q, C, G, P, A, V, I, L, M, F, Y, or W; X₃ is R, H, K, D, E, S, T, N, Q, C, G, P, A, V, I, L, M, F, Y, or W; X₄ is R, H, K, D, E, S, T, N, Q, C, G, P, A, V, I, L, M, F, Y, or W; X₅ is R, H, K, D, E, S, T, N, Q, C, G, P, A, V, I, L, M, F, Y, or W; X₆ is R, H, K, D, E, S, T, N, Q, C, G, P, A, V, I, L, M, F, Y, or W; X₇ is R, H, K, D, E, S, T, N, Q, C, G, P, A, V, I, L, M, F, Y, or W; X₈ is R, H, K, D, E, S, T, N, Q, C, G, P, A, V, I, L, M, F, Y, or W; X₉ is R, H, K, D, E, S, T, N, Q, C, G, P, A, V, I, L, M, F, Y, or W; and X₁₀ is absent. For example, an “AVP” can refer to a polypeptide with the amino acid sequence “KACCPCYWGGCPWGAACYPAGCAAAK” (e.g., SEQ ID NO:30). The term “AVP” refers to monomers or polymers of AVP, e.g., AVP refers to homopolymers or heteropolymers of two or more AVP polypeptides, wherein the amino acid sequence of each AVP is the same or different. And, AVP refers to a fused protein comprising two or more AVP polypeptides separated by a cleavable or non-cleavable linker, and wherein the amino acid sequence of each AVP may be the same or different.

“BAAS” means barley alpha-amylase signal peptide, and is an example of an ERSP. One example of a BAAS is a BAAS having the amino acid sequence of SEQ ID NO:5 (NCBI Accession No. AAA32925.1).

“Biomass” refers to any measured plant product.

“Binary vector” or “binary expression vector” means an expression vector which can replicate itself in both E. coli strains and Agrobacterium strains. Also, the vector contains a region of DNA (often referred to as t-DNA) bracketed by left and right border sequences that is recognized by virulence genes to be copied and delivered into a plant cell by Agrobacterium.

Bt proteins” and “Bt peptides” are used interchangeably and include peptides produced by Bt are collectively referred to herein as Bt toxic proteins or “Bt TPs”. Such peptides are frequently written as “cry”, “cyt” or “VIP” proteins encoded by the cry, cyt and vip genes. Bt TPs are more usually attributed to insecticidal crystal proteins encoded by the cry genes.

“C-terminal” refers to the free carboxyl group (i.e., —COOH) that is positioned on the terminal end of a polypeptide.

“Cleavable Linker” see Linker.

“Cloning” refers to the process and/or methods concerning the insertion of a DNA segment (e.g., usually a gene of interest, for example avp) from one source and recombining it with a DNA segment from another source (e.g., usually a vector, for example, a plasmid) and directing the recombined DNA, or “recombinant DNA” to replicate, usually by transforming the recombined DNA into a bacteria or yeast host.

“Conditioned medium” means the cell culture medium which has been used by cells and is enriched with cell derived materials but does not contain cells.

“Core” or “AVP-Core” or “AVP-Core polypeptide” or “AVP-Core synthetic polypeptide” refers to one or more AVP polypeptides with variable element residues at certain positions throughout the amino acid sequence. For example, in one embodiment an AVP-Core polypeptide is “AVP-Core-5” or “Core 5” comprising the amino acid sequence “KACCPCYWGGCPWGAACYPAGCAAAK” (SEQ ID NO:30). Another example of an AVP-Core polypeptide is “AVP-Core-4” or “Core 4,” comprising the amino acid sequence “KACCPCYWAACPWAAACYAAACAAAK” (SEQ ID NO:31). Yet another example of an AVP-Core polypeptide is “AVP-Core-3” or “Core 3,” comprising the amino acid sequence “KPYWPWYK” (SEQ ID NO:32). A further example of an AVP-Core polypeptide is “AVP-Core-2” or “Core 2,” comprising the amino acid sequence “KPYWPWYKV” (SEQ ID NO:33). In another example, “AVP-Core-1” or “Core 1” with the amino acid sequence “PYWPWY” (SEQ ID NO:34).

“Defined medium” means a medium that is composed of known chemical components but does not contain crude proteinaceous extracts or by-products such as yeast extract or peptone.

“Double expression cassette” refers to two AVP expression cassette s contained on the same vector.

“Disulfide bond” means a covalent bond between two cysteine amino acids derived by the coupling of two thiol groups on their side chains.

“Double transgene peptide expression vector” or “double transgene expression vector” means a yeast expression vector which contains two copies of the Av3 peptide expression cassette.

“DNA” refers to deoxyribonucleic acid, comprising a polymer of one or more deoxyribonucleotides or nucleotides (i.e., adenine [A], guanine [G], thymine [T], or cytosine [C]), which can be arranged in single-stranded or double-stranded form. For example, one or more nucleotides creates a polynucleotide.

“dNTPs” refers to the nucleoside triphosphates that compose DNA and RNA.

“ELISA” or “iELISA” means a molecular biology protocol in which the samples are fixed to the surface of a plate and then detected as follows: a primary antibody is applied followed by a secondary antibody conjugated to an enzyme which converts a colorless substrate to colored substrate which can be detected and quantified across samples. During the protocol, antibodies are washed away such that only those that bind to their epitopes remain for detection. The samples, in our hands, are proteins isolated from plants, and ELISA allows for the quantification of the amount of expressed transgenic protein recovered.

“Enhancer element” refers to a DNA sequence operably linked to a promoter, which can exert increased transcription activity on the promoter relative to the transcription activity that results from the promoter in the absence of the enhancer element.

“Expression cassette” refers to a segment of DNA that contains one or more (1) promoter and/or enhancer elements; (2) an appropriate mRNA stabilizing polyadenylation signal; and/or (3) the DNA sequence of interest, for example, an Av3 variant polynucleotide sequence. Additional elements that can included in an expression cassette are cis-acting elements such as an internal ribosome entry site (IRES); introns; and posttranscriptional regulatory elements.

“Expression ORF” means a nucleotide encoding a protein complex and is defined as the nucleotides in the ORF.

“ER” or “Endoplasmic reticulum” is a subcellular organelle common to all eukaryotes where some post translation modification processes occur.

“ERSP” or “Endoplasmic reticulum signal peptide” is an N-terminus sequence of amino acids that during protein translation of the mRNA molecule encoding an Av3 protein is recognized and bound by a host cell signal-recognition particle, which moves the protein translation ribosome/mRNA complex to the ER in the cytoplasm. The result is the protein translation is paused until it docks with the ER where it continues and the resulting protein is injected into the ER.

“ER trafficking” means transportation of a cell expressed protein into ER for post-translational modification, sorting and transportation.

“FECT” means a transient plant expression system using Foxtail mosaic virus with elimination of coating protein gene and triple gene block.

“GFP” means a green fluorescent protein from the jellyfish Aequorea victoria.

“Insect” includes all organisms in the class “Insecta”. The term “pre-adult” insects refers to any form of an organism prior to the adult stage, including, for example, eggs, larvae, and nymphs.

As used herein, the term “insecticidal” is generally used to refer to the ability of a polypeptide or protein used herein, to increase mortality or inhibit growth rate of insects. As used herein, the term “nematicidal” refers to the ability of a polypeptide or protein used herein, to increase mortality or inhibit the growth rate of nematodes. In general, the term “nematode” comprises eggs, larvae, juvenile and mature forms of said organism.

“Insecticidal protein” refers to any protein and/or polypeptide amino acid sequence, configuration, or arrangement, comprising one or more AVPs. For example, an insecticidal protein can refer to an Av3 variant peptide; or an AVP fused with one or more proteins such as a stabilizing domain (STA); an endoplasmic reticulum signaling protein (ERSP); an insect cleavable or insect non-cleavable linker, or an Av3 variant peptide fused to one or more Av3 variant peptides; and/or any other combination therefor.

“Insecticidal activity” means that on or after exposure of the insect to compounds or peptides, the insect either dies stops or slows its movement or its feeding, stops or slows its growth, fails to pupate, cannot reproduce or cannot produce fertile offspring.

“Integrative expression vector” or “integrative vector” means a yeast expression vector which can insert itself into a specific locus of the yeast cell genome and stably becomes a part of the yeast genome.

“Knockdown dose 50” or “KD₅₀” refers to the median dose required to cause paralysis or cessation of movement in 50% of a population, for example a population of Musca domestica (common housefly) and/or Aedes aegypti (mosquito).

“LAC4 promoter” refers to a DNA segment comprised of the promoter sequence derived from the K. lactis β-galactosidase gene. The LAC4 promoters is strong and inducible reporter that is used to drive expression of exogenous genes transformed into yeast.

“LAC4 terminator” refers to a DNA segment comprised of the transcriptional terminator sequence derived from the K. lactis β-galactosidase gene.

“LD₅₀” refers to lethal dose 50 which means the dose required to kill 50% of a population.

“Linker, Cleavable Linker, or Peptide Linker” means a short peptide sequence (a binary or tertiary peptide) that is the target site of at least two types of proteases one of which is an insect and/or nematode protease and the other one of which is a human protease such that the linker can be separated by both types of protease that can cleave and separate the protein into two parts or a short DNA sequence that is placed in the reading frame in the ORF and encoding a short peptide sequence in the protein that is the target site of an insect and/or nematode and an animal (e.g. human) protease that can cleave and separate the protein into two parts.

“Motif” refers to a polynucleotide or polypeptide sequence that is implicated in having some biological significance and/or exerts some effect or is involved in some biological process.

“Multiple cloning site” or “MCS” refers to a segment of DNA found on a vector that contains numerous restriction sites in which a DNA sequence of interest can be inserted.

“Mutant” refers to an organism, DNA sequence, or polypeptide sequence, that has an alteration (for example, in the DNA sequence), which causes said organism and/or sequence to be different from the naturally occurring or wild-type organism and/or sequence. For example, a mutant Av3 polypeptide can possess an alteration to its peptide composition resulting in a non-naturally occurring Av3 polypeptide.

“N-terminal” refers to the free amine group (i.e., —NH₂) that is positioned on beginning or start of a polypeptide.

“One letter code” means the peptide sequence which is listed in its one letter code to distinguish the various amino acids in the primary structure of a protein: alanine=A, arginine=R, asparagine=N, aspartic acid=D, asparagine or aspartic acid=B, cysteine=C, glutamic acid=E, glutamine=Q, glutamine or glutamic acid=Z, glycine=G, histidine=H, isoleucine=I, leucine=L, lysine=K, methionine=M, phenylalanine=F, proline=P, serine=S, threonine=T, tryptophan=W, tyrosine=Y, and valine=V.

“ORF” or “Open reading frame” or “peptide expression ORF” means that DNA sequence encoding a protein which begins with an ATG start codon and ends with a TGA, TAA or TAG stop codon. ORF can also mean the translated protein that the DNA encodes.

“Operably linked” means that the two adjacent DNA sequences are placed together such that the transcriptional activation of one can act on the other. “Operably linked” with regard to peptide and/or polypeptide molecules means that two or more peptide and/or polypeptide molecules are connected in such a way as to yield a single polypeptide chain, or connected in such a way inasmuch that one peptide exerts some effect on the other.

“Peptide expression cassette”, or “expression cassette” means a DNA sequence which is composed of all the DNA elements necessary to complete transcription of an insecticidal peptide in a biological expression system. In the described methods herein, it includes a transcription promoter, a DNA sequence to encode an α-mating factor signal sequence and a Kex2 cleavage site, an insecticidal peptide transgene, a stop codon and a transcription terminator.

“Peptide expression vector” means a host organism expression vector which contains a heterologous peptide transgene.

“Peptide expression yeast strain”, “peptide expression strain” or “peptide production strain” means a yeast strain which can produce a heterologous peptide.

“Peptide Linker” see Linker.

“Peptide transgene” or “insecticidal peptide transgene” or “Av3 peptide transgene” means a DNA sequence that encodes an Av3 peptide and can be translated in a biological expression system.

“Peptide yield” means the insecticidal peptide concentration in the conditioned medium which is produced from the cells of a peptide expression yeast strain. It can be represented by the mass of the produced peptide in a unit of volume, for example, mg per liter or mg/L, or by the UV absorbance peak area of the produced peptide in the HPLC chromatograph, for example, mAu·sec.

“Pest” includes, but is not limited to: insects, fungi, bacteria, nematodes, mites, ticks, and the like.

“Pesticidally-effective amount” refers to an amount of the pesticide that is able to bring about death to at least one pest, or to noticeably reduce pest growth, feeding, or normal physiological development. This amount will vary depending on such factors as, for example, the specific target pests to be controlled, the specific environment, location, plant, crop, or agricultural site to be treated, the environmental conditions, and the method, rate, concentration, stability, and quantity of application of the pesticidally-effective polypeptide composition. The formulations may also vary with respect to climatic conditions, environmental considerations, and/or frequency of application and/or severity of pest infestation.

“Plant transgenic protein” means a protein from a heterologous species that is expressed in a plant after the DNA or RNA encoding it was delivered into one or more of the plant cells.

“Plant” shall mean whole plants, plant organs (e.g., leaves, stems, roots, etc.), seeds, plant cells, propagules, embryos and progeny of the same. Plant cells can be differentiated or undifferentiated (e.g. callus, suspension culture cells, protoplasts, leaf cells, root cells, phloem cells, and pollen).

“Plasmid” refers to a DNA segment that acts as a carrier for a gene of interest (e.g., avp) and, when transformed or transfected into an organism, can replicate and express the DNA sequence contained within the plasmid independently of the host organism. Plasmids are a type of vector, and can be “cloning vectors” (i.e., simple plasmids used to clone a DNA fragment and/or select a host population carrying the plasmid via some selection indicator) or “expression plasmids” (i.e., plasmids used to produce large amounts of polynucleotides and/or polypeptides).

“Post-transcriptional regulatory elements” are DNA segments and/or mechanisms that affect mRNA after it has been transcribed. Mechanisms of post-transcriptional mechanisms include splicing events; capping, splicing, and addition of a Poly (A) tail, and other mechanisms known to those having ordinary skill in the art.

“Promoter” refers to a region of DNA to which RNA polymerase binds and initiates the transcription of a gene.

“Protein” has the same meaning as “Peptide” in this document.

“Recombinant DNA” or “rDNA” refers to DNA that is comprised of two or more different DNA segments.

“Recombinant vector” means a DNA plasmid vector into which foreign DNA has been inserted.

“Regulatory elements” refers to promoters; enhancers; internal ribosomal entry sites (IRES); polyadenylation signals; poly-U sequences; and/or other elements that influence gene expression, for example, in a tissue-specific manner; temporal-dependent manner; to increase or decrease expression; and/or to cause constitutive expression.

“Restriction enzyme” or “restriction endonuclease” refers to an enzyme that cleaves DNA at a specified restriction site. For example, a restriction enzyme can cleave a plasmid at an EcoRI, SacII or BstXI restriction site allowing the plasmid to be linearized, and the DNA of interest to be ligated.

“Restriction site” refers to a location on DNA comprising a sequence of 4 to 8 nucleotides, and whose sequence is recognized by a particular restriction enzyme.

“Selection gene” means a gene which confers an advantage for a genomically modified organism to grow under the selective pressure.

“Subcloning” or “subcloned” refers to the process of transferring DNA from one vector to another, usually advantageous vector. For example, polynucleotide encoding a mutant Av3 polypeptide can be subcloned into a pLB102 plasmid subsequent to selection of yeast colonies transformed with pKLAC1 plasmids.

“SSI” or “site-specific integration” refers to the process directing a transgene to a target site in a host-organism's genome; thus, SSI allows the integration of genes of interest into pre-selected genome locations of a host-organism.

“STA”, or “Translational stabilizing protein”, or “stabilizing domain”, or “stabilizing protein”, (used interchangeably herein) means a protein with sufficient tertiary structure that it can accumulate in a cell without being targeted by the cellular process of protein degradation. The protein can be between 5 and 50 amino acids (aa). The translational stabilizing protein is coded by a DNA sequence for a protein that is fused in frame with a sequence encoding an insecticidal protein or an Av3 peptide in the ORF. The fusion protein can either be upstream or downstream of the toxic protein and can have any intervening sequence between the two sequences as long as the intervening sequence does not result in a frame shift of either DNA sequence. The translational stabilizing protein can also have an activity which increases delivery of the AVP across the gut wall and into the hemolymph of the insect.

“Structural motif” refers to the three-dimensional arrangement of polypeptides, and/or the arrangement of operably linked polypeptide segments. For example, the polypeptide comprising ERSP-STA-L-AVP has an ERSP motif, an STA motif, a LINKER motif, and an AVP polypeptide.

“Transfection” refers to the process wherein exogenous DNA (e.g., a vector containing a polynucleotide that encodes an AVP) is inserted into a host cell (i.e., a eukaryote) resulting in a transgenic or genetically modified organism (GMO) that is able to express the exogenous DNA. As used herein, when neither bacterial nor eukaryotic host is indicated, the term “transfection” is used synonymously with “transformation.” As used herein, the term “transfected” or “transfection” may refer to the process of introducing foreign DNA into a host cell or organism, and the resulting organism may be referred to as being “transfected” or “transformed.” The term “transfected” as used herein refers mainly to the introduction of exogenous DNA to animal cells or yeast cells.

“Transformation” refers to the process wherein exogenous DNA (e.g., a vector containing a polynucleotide that encodes an AVP) is inserted into a host cell (i.e., a bacteria, or a yeast cell) resulting in a transgenic or genetically modified organism (GMO) that is able to express the exogenous DNA. As used herein, when neither bacterial nor eukaryotic host is indicated, the term “transformation” is used synonymously with “transfection.” The term “transformation” as used herein refers to the introduction of exogenous DNA to bacteria and non-animal eukaryotes.

“Transgene” means a heterologous DNA sequence encoding a protein which is transformed into a plant.

“Transgenic host cell” means a cell which is transformed with a gene and has been selected for its transgenic status via an additional selection gene.

“Transgenic plant” means a plant that has been derived from a single cell that was transformed with foreign DNA such that every cell in the plant contains that transgene.

“Transient expression system” means an Agrobacterium tumefaciens-based system which delivers DNA encoding a disarmed plant virus into a plant cell where it is expressed. The plant virus has been engineered to express a protein of interest at high concentrations, up to 40% of the TSP.

“Triple expression cassette refers to three AVP expression cassette s contained on the same vector.

“TRBO” means a transient plant expression system using Tobacco mosaic virus with removal of the viral coating protein gene.

“TSP” or “total soluble protein” means the total amount of protein that can be extracted from a plant tissue sample and solubilized into the extraction buffer.

“Vector” refers to the DNA segment that accepts a foreign gene of interest (e.g., avp). The gene of interest is known as an “insert” or “transgene”.

“Wild type” or “WT” refers to the phenotype and/or genotype (i.e., the appearance or sequence) of an organism, polynucleotide sequence, and/or polypeptide sequence, as it is found and/or observed in its naturally occurring state or condition.

“Yeast expression vector,” or “expression vector”, or “vector,” means a plasmid which can introduce a heterologous gene and/or expression cassette into yeast cells to be transcribed and translated.

“Yield” refers to the production of a peptide, and increased yields can mean increased amounts of production, increased rates of production, and an increased average or median yield and increased frequency at higher yields. The term “yield” when used in reference to plant crop growth and/or production, as in “yield of the plant” refers to the quality and/or quantity of biomass produced by the plant.

Throughout this specification, unless specifically stated otherwise or the context requires otherwise, reference to a single step, composition of matter, group of steps or group of compositions of matter shall be taken to encompass one and a plurality (i.e., one or more) of those steps, compositions of matter, groups of steps or group of compositions of matter.

The present disclosure is performed without undue experimentation using, unless otherwise indicated, conventional techniques of molecular biology, microbiology, virology, recombinant DNA technology, solid phase and liquid nucleic acid synthesis, peptide synthesis in solution, solid phase peptide synthesis, immunology, cell culture, and formulation. Such procedures are described, for example, in Sambrook, Fritsch & Maniatis, Molecular Cloning: A Laboratory Manual, Cold Spring Harbor Laboratories, New York, Second Edition (1989), whole of Vols I, II, and III; DNA Cloning: A Practical Approach, Vols. I and II (D. N. Glover, ed., 1985), IRL Press, Oxford, whole of text; Oligonucleotide Synthesis: A Practical Approach (M. J. Gait, ed, 1984) IRL Press, Oxford, whole of text, and particularly the papers therein by Gait, pp1-22; Atkinson et al, pp35-81; Sproat et al, pp 83-115; and Wu et al, pp 135-151; 4. Nucleic Acid Hybridization: A Practical Approach (B. D. Hames & S. J. Higgins, eds., 1985) IRL Press, Oxford, whole of text; Immobilized Cells and Enzymes: A Practical Approach (1986) IRL Press, Oxford, whole of text; Perbal, B., A Practical Guide to Molecular Cloning (1984); Methods In Enzymology (S. Colowick and N. Kaplan, eds., Academic Press, Inc.), whole of series; J. F. Ramalho Ortigao, “The Chemistry of Peptide Synthesis” In: Knowledge database of Access to Virtual Laboratory website (Interactiva, Germany); Sakakibara, D., Teichman, J., Lien, E. Land Fenichel, R. L. (1976). Biochem. Biophys. Res. Commun. 73 336-342; Merrifield, R. B. (1963). J. Am. Chem. Soc. 85, 2149-2154; Barany, G. and Merrifield, R. B. (1979) in The Peptides (Gross, E. and Meienhofer, 3. eds.), vol. 2, pp. 1-284, Academic Press, New York. 12. Wiinsch, E., ed. (1974) Synthese von Peptiden in Houben-Weyls Metoden der Organischen Chemie (Muler, E., ed.), vol. 15, 4th edn., Parts 1 and 2, Thieme, Stuttgart; Bodanszky, M. (1984) Principles of Peptide Synthesis, Springer-Verlag, Heidelberg; Bodanszky, M. & Bodanszky, A. (1984) The Practice of Peptide Synthesis, Springer-Verlag, Heidelberg; Bodanszky, M. (1985) Int. J. Peptide Protein Res. 25, 449-474; Handbook of Experimental Immunology, Vols. I-IV (D. M. Weir and C. C. Blackwell, eds., 1986, Blackwell Scientific Publications); and Animal Cell Culture: Practical Approach, Third Edition (John R. W. Masters, ed., 2000); each of these references are incorporated herein by reference in their entireties.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

### AVP

The sea anemone, Anemonia viridis, possesses a variety of toxins that it uses to defend itself: one of these toxins is the neurotoxin “Av3.” Av3 is a type III sea anemone toxin that inhibits the inactivation of voltage-gated sodium (Na⁺) channels at receptor site 3, resulting in contractile paralysis. The binding of an Av3 toxin to site 3 results in the inactivated state of the sodium channel to become destabilized, which in turn causes the channel to remain in the open position (see Blumenthal et al., Voltage-gated sodium channel toxins: poisons, probes, and future promise. Cell Biochem Biophys. 2003; 38(2):215-38). Av3 shows high selectivity for crustacean and insect sodium channels, and low selectivity for mammalian sodium channels (see Moran et al., Sea anemone toxins affecting voltage-gated sodium channels—molecular and evolutionary features, Toxicon. 2009 Dec. 15; 54(8): 1089-1101). An exemplary Av3 polypeptide from Anemonia viridis is provided having the amino acid sequence of SEQ ID NO:1.

In some embodiments, AVP Av3a, has an N-terminal amino acid substitution of R1K relative to SEQ ID NO:1, changing the polypeptide sequence from the wild-type “RSCCPCYWGGCPWGQNCYPEGCSGPKV” (SEQ ID NO: 1) to “KSCCPCYWGGCPWGQNCYPEGCSGPKV” (SEQ ID NO:2). The term “AVPa” or Av3a” refers to those embodiments of an AVP that have an N-terminal amino acid substitution of R1K relative to SEQ ID NO: 1.

In some embodiments, AVP Av3a-C1 has a C-terminal mutation. For example, in some embodiments, the C-terminal amino acid can be deleted relative to SEQ ID NO:1, changing the polypeptide sequence from the wild-type “RSCCPCYWGGCPWGQNCYPEGCSGPKV” (SEQ ID NO: 1) to “RSCCPCYWGGCPWGQNCYPEGCSGPK” (SEQ ID NO:3). The term “AVPa-C1” or Av3a-C1” refers to those embodiments of an AVP that have a C-terminal amino acid deletion relative to SEQ ID NO:1.

In some embodiments, an AVP can have an N-terminal mutation and a C-terminal mutation. For example, in some embodiments, the N-terminal amino acid can have a substitution of R1K relative to SEQ ID NO: 1, and the C-terminal amino acid can be deleted relative to SEQ ID NO:1, changing the polypeptide sequence from the wild-type “RSCCPCYWGGCPWGQNCYPEGCSGPKV” (SEQ ID NO: 1) to “KSCCPCYWGGCPWGQNCYPEGCSGPK” (SEQ ID NO:4). The term “AVPb” or Av3b” refers to those embodiments that have an N-terminal amino acid substitution of R1K relative to SEQ ID NO:1, and a C-terminal amino acid deleted relative to SEQ ID NO:1.

In some embodiments, an AVP can have an amino acid sequence comprising the following: X₁-X₂-C-C-P-C-Y-W-G-G-C-P-W-G-X₃-X₄-C-Y-P-X₅-G-C-X₆-X₇-X₈-X₉-X₁₀ (SEQ ID NO: 35); wherein X₁ is H, K, D, E, S, T, N, Q, C, G, P, A, V, I, L, M, F, Y, or W; X₂ is R, H, K, D, E, S, T, N, Q, C, G, P, A, V, I, L, M, F, Y, or W; X₃ is R, H, K, D, E, S, T, N, Q, C, G, P, A, V, I, L, M, F, Y, or W; X₄ is R, H, K, D, E, S, T, N, Q, C, G, P, A, V, I, L, M, F, Y, or W; X₅ is R, H, K, D, E, S, T, N, Q, C, G, P, A, V, I, L, M, F, Y, or W; X₆ is R, H, K, D, E, S, T, N, Q, C, G, P, A, V, I, L, M, F, Y, or W; X₇ is R, H, K, D, E, S, T, N, Q, C, G, P, A, V, I, L, M, F, Y, or W; X₈ is R, H, K, D, E, S, T, N, Q, C, G, P, A, V, I, L, M, F, Y, or W; X₉ is R, H, K, D, E, S, T, N, Q, C, G, P, A, V, I, L, M, F, Y, or W; and X₁₀ is R, H, K, D, E, S, T, N, Q, C, G, P, A, V, I, L, M, F, Y, W or absent.

In some embodiments, an AVP can have an amino acid sequence comprising the following: X₁-X₂-C-C-P-C-Y-W-G-G-C-P-W-G-X₃-X₄-C-Y-P-X₅-G-C-X₆-X₇-X₈-X₉-X₁₀ (SEQ ID NO: 36); wherein X₁ is R, H, K, D, E, S, T, N, Q, C, G, P, A, V, I, L, M, F, Y, or W; X₂ is R, H, K, D, E, S, T, N, Q, C, G, P, A, V, I, L, M, F, Y, or W; X₃ is R, H, K, D, E, S, T, N, Q, C, G, P, A, V, I, L, M, F, Y, or W; X₄ is R, H, K, D, E, S, T, N, Q, C, G, P, A, V, I, L, M, F, Y, or W; X₅ is R, H, K, D, E, S, T, N, Q, C, G, P, A, V, I, L, M, F, Y, or W; X₆ is R, H, K, D, E, S, T, N, Q, C, G, P, A, V, I, L, M, F, Y, or W; X₇ is R, H, K, D, E, S, T, N, Q, C, G, P, A, V, I, L, M, F, Y, or W; X₈ is R, H, K, D, E, S, T, N, Q, C, G, P, A, V, I, L, M, F, Y, or W; X₉ is R, H, K, D, E, S, T, N, Q, C, G, P, A, V, I, L, M, F, Y, or W; and X₁₀ is absent.

In some embodiments, an AVP can have an amino acid sequence of “KACCPCYWGGCPWGAACYPAGCAAAK” (e.g., SEQ ID NO:30).

In various embodiments, polynucleotides encoding insecticidal proteins can be used to transform plant cells. In some embodiments, the insecticidal transgenic proteins may be formulated into compositions that can be sprayed or otherwise applied in any manner known to those skilled in the art to the surface of plants or parts thereof. Accordingly, DNA constructs are provided herein, operable to encode one or more insecticidal transgenic proteins under the appropriate conditions in a host cell, for example, a plant cell. Methods for controlling a pest infection by a parasitic insect of a plant cell comprises administering or introducing a polynucleotide encoding an insecticidal transgenic protein as described herein to a plant, plant tissue, or a plant cell by recombinant techniques and growing said recombinantly altered plant, plant tissue or plant cell in a field exposed to the pest. Alternatively, the insecticidal transgenic protein can be formulated into a sprayable composition and applied directly to susceptible plants by direct application, such that upon ingestion of the insecticidal transgenic protein by the infectious insect, one or more copies or monomers of the insecticidal peptide is cleaved from the insecticidal protein ingested by the infectious insect and produces its effect to destroy the insect.

In some embodiments, an AVP can be a homopolymer or of two or more AVP polypeptides, wherein the amino acid sequence of each AVP is the same or different. For example, in some embodiments, an AVP can have one polypeptide comprising an N-terminal mutation replacing the amino terminal Arginine with Lysine (R1K) amino acid relative to SEQ ID NO:1, linked to another polypeptide comprising an N-terminal mutation replacing the amino terminal Arginine with Lysine (R1K) amino acid relative to SEQ ID NO:1.

In some embodiments, an AVP can be a heteropolymer or of two or more AVP polypeptides, wherein the amino acid sequence of each AVP is the same or different. For example, in some embodiments, a first AVP polymer can have an N-terminal mutation replacing the amino terminal Arginine with Lysine (R1K) amino acid relative to SEQ ID NO:1, or a deletion of the C-terminal valine amino acid relative to SEQ ID NO:1; and a second AVP polymer can have an amino acid sequence X₁-X₂-C-C-P-C-Y-W-G-G-C-P-W-G-X₃-X₄-C-Y-P-X₅-G-C-X₆-X₇-X₈-X₉-X₁₀ (SEQ ID NO: 35); wherein X₁ is H, K, D, E, S, T, N, Q, C, G, P, A, V, I, L, M, F, Y, or W; X₂ is R, H, K, D, E, S, T, N, Q, C, G, P, A, V, I, L, M, F, Y, or W; X₃ is R, H, K, D, E, S, T, N, Q, C, G, P, A, V, I, L, M, F, Y, or W; X₄ is R, H, K, D, E, S, T, N, Q, C, G, P, A, V, I, L, M, F, Y, or W; X₅ is R, H, K, D, E, S, T, N, Q, C, G, P, A, V, I, L, M, F, Y, or W; X₆ is R, H, K, D, E, S, T, N, Q, C, G, P, A, V, I, L, M, F, Y, or W; X₇ is R, H, K, D, E, S, T, N, Q, C, G, P, A, V, I, L, M, F, Y, or W; X₈ is R, H, K, D, E, S, T, N, Q, C, G, P, A, V, I, L, M, F, Y, or W; X₉ is R, H, K, D, E, S, T, N, Q, C, G, P, A, V, I, L, M, F, Y, or W; and X₁₀ is R, H, K, D, E, S, T, N, Q, C, G, P, A, V, I, L, M, F, Y, W or absent; or an amino acid sequence X₁-X₂-C-C-P-C-Y-W-G-G-C-P-W-G-X₃-X₄-C-Y-P-X₅-G-C-X₆-X₇-X₈-X₉-X₁₀ (SEQ ID NO: 36); wherein X₁ is R, H, K, D, E, S, T, N, Q, C, G, P, A, V, I, L, M, F, Y, or W; X₂ is R, H, K, D, E, S, T, N, Q, C, G, P, A, V, I, L, M, F, Y, or W; X₃ is R, H, K, D, E, S, T, N, Q, C, G, P, A, V, I, L, M, F, Y, or W; X₄ is R, H, K, D, E, S, T, N, Q, C, G, P, A, V, I, L, M, F, Y, or W; X₅ is R, H, K, D, E, S, T, N, Q, C, G, P, A, V, I, L, M, F, Y, or W; X₆ is R, H, K, D, E, S, T, N, Q, C, G, P, A, V, I, L, M, F, Y, or W; X₇ is R, H, K, D, E, S, T, N, Q, C, G, P, A, V, I, L, M, F, Y, or W; X₈ is R, H, K, D, E, S, T, N, Q, C, G, P, A, V, I, L, M, F, Y, or W; X₉ is R, H, K, D, E, S, T, N, Q, C, G, P, A, V, I, L, M, F, Y, or W; and X₁₀ is absent.

In some embodiments, the AVP can be a fused protein comprising two or more AVP polypeptides separated by a cleavable or non-cleavable linker, and wherein the amino acid sequence of each AVP may be the same or different. For example, in some embodiments, a first AVP polymer can have an N-terminal mutation replacing the amino terminal Arginine with Lysine (RIK) amino acid relative to SEQ ID NO:1, or a deletion of the C-terminal valine amino acid relative to SEQ ID NO: 1; that is fused a second AVP polymer can have an amino acid sequence X₁-X₂-C-C-P-C-Y-W-G-G-C-P-W-G-X₃-X₄-C-Y-P-X₅-G-C-X₆-X₇-X₈-X₉-X₁₀ (SEQ ID NO: 35); wherein X₁ is H, K, D, E, S, T, N, Q, C, G, P, A, V, I, L, M, F, Y, or W; X₂ is R, H, K, D, E, S, T, N, Q, C, G, P, A, V, I, L, M, F, Y, or W; X₃ is R, H, K, D, E, S, T, N, Q, C, G, P, A, V, I, L, M, F, Y, or W; X₄ is R, H, K, D, E, S, T, N, Q, C, G, P, A, V, I, L, M, F, Y, or W; X₈ is R, H, K, D, E, S, T, N, Q, C, G, P, A, V, I, L, M, F, Y, or W; X₆ is R, H, K, D, E, S, T, N, Q, C, G, P, A, V, I, L, M, F, Y, or W; X₇ is R, H, K, D, E, S, T, N, Q, C, G, P, A, V, I, L, M, F, Y, or W; X₈ is R, H, K, D, E, S, T, N, Q, C, G, P, A, V, I, L, M, F, Y, or W; X₉ is R, H, K, D, E, S, T, N, Q, C, G, P, A, V, I, L, M, F, Y, or W; and X₁₀ is R, H, K, D, E, S, T, N, Q, C, G, P, A, V, I, L, M, F, Y, W or absent; or an amino acid sequence X₁-X₂-C-C-P-C-Y-W-G-G-C-P-W-G-X₃-X₄-C-Y-P-X₅-G-C-X₆-X₇-X₈-X₉-X₁₀ (SEQ ID NO: 36); wherein X₁ is R, H, K, D, E, S, T, N, Q, C, G, P, A, V, I, L, M, F, Y, or W; X₂ is R, H, K, D, E, S, T, N, Q, C, G, P, A, V, I, L, M, F, Y, or W; X₃ is R, H, K, D, E, S, T, N, Q, C, G, P, A, V, I, L, M, F, Y, or W; X₄ is R, H, K, D, E, S, T, N, Q, C, G, P, A, V, I, L, M, F, Y, or W; X₅ is R, H, K, D, E, S, T, N, Q, C, G, P, A, V, I, L, M, F, Y, or W; X₆ is R, H, K, D, E, S, T, N, Q, C, G, P, A, V, I, L, M, F, Y, or W; X₇ is R, H, K, D, E, S, T, N, Q, C, G, P, A, V, I, L, M, F, Y, or W; X₈ is R, H, K, D, E, S, T, N, Q, C, G, P, A, V, I, L, M, F, Y, or W; X₉ is R, H, K, D, E, S, T, N, Q, C, G, P, A, V, I, L, M, F, Y, or W; and X₁₀ is absent; with said polypeptides separated by a cleavable linker.

In some embodiments, the AVP can be a fused protein comprising two or more AVP polypeptides separated by a cleavable or non-cleavable linker, and wherein the amino acid sequence of each AVP may be the same or different. For example, in some embodiments, a first AVP polymer can have an N-terminal mutation replacing the amino terminal Arginine with Lysine (RIK) amino acid relative to SEQ ID NO:1, or a deletion of the C-terminal valine amino acid relative to SEQ ID NO: 1; that is fused a second AVP polymer can have an amino acid sequence X₁-X₂-C-C-P-C-Y-W-G-G-C-P-W-G-X₃-X₄-C-Y-P-X₅-G-C-X₆-X₇-X₈-X₉-X₁₀ (SEQ ID NO: 35); wherein X₁ is H, K, D, E, S, T, N, Q, C, G, P, A, V, I, L, M, F, Y, or W; X₂ is R, H, K, D, E, S, T, N, Q, C, G, P, A, V, I, L, M, F, Y, or W; X₃ is R, H, K, D, E, S, T, N, Q, C, G, P, A, V, I, L, M, F, Y, or W; X₄ is R, H, K, D, E, S, T, N, Q, C, G, P, A, V, I, L, M, F, Y, or W; X₅ is R, H, K, D, E, S, T, N, Q, C, G, P, A, V, I, L, M, F, Y, or W; X₆ is R, H, K, D, E, S, T, N, Q, C, G, P, A, V, I, L, M, F, Y, or W; X₇ is R, H, K, D, E, S, T, N, Q, C, G, P, A, V, I, L, M, F, Y, or W; X₈ is R, H, K, D, E, S, T, N, Q, C, G, P, A, V, I, L, M, F, Y, or W; X₉ is R, H, K, D, E, S, T, N, Q, C, G, P, A, V, I, L, M, F, Y, or W; and X₁₀ is R, H, K, D, E, S, T, N, Q, C, G, P, A, V, I, L, M, F, Y, W or absent; or an amino acid sequence X₁-X₂-C-C-P-C-Y-W-G-G-C-P-W-G-X₃-X₄-C-Y-P-X₅-G-C-X₆-X₇-X₈-X₉-X₁₀ (SEQ ID NO: 36); wherein X₁ is R, H, K, D, E, S, T, N, Q, C, G, P, A, V, I, L, M, F, Y, or W; X₂ is R, H, K, D, E, S, T, N, Q, C, G, P, A, V, I, L, M, F, Y, or W; X₃ is R, H, K, D, E, S, T, N, Q, C, G, P, A, V, I, L, M, F, Y, or W; X₄ is R, H, K, D, E, S, T, N, Q, C, G, P, A, V, I, L, M, F, Y, or W; X₅ is R, H, K, D, E, S, T, N, Q, C, G, P, A, V, I, L, M, F, Y, or W; X₆ is R, H, K, D, E, S, T, N, Q, C, G, P, A, V, I, L, M, F, Y, or W; X₇ is R, H, K, D, E, S, T, N, Q, C, G, P, A, V, I, L, M, F, Y, or W; X₈ is R, H, K, D, E, S, T, N, Q, C, G, P, A, V, I, L, M, F, Y, or W; X₉ is R, H, K, D, E, S, T, N, Q, C, G, P, A, V, I, L, M, F, Y, or W; and X₁₀ is absent; with said polypeptides separated by a non-cleavable linker.

In some embodiments, the AVP can be a fused protein comprising two or more AVP polypeptides separated by a cleavable or non-cleavable linker, and wherein the amino acid sequence of each AVP may be the same or different, wherein the linker is cleavable inside the gut or hemolymph of an insect.

Exemplary methods for the generation of cleavable and non-cleavable linkers can be found in U.S. patent application Ser. No. 15/727,277, filed Oct. 6, 2017; and PCT Application No. PCT/US2013/030042, filed Mar. 8, 2013, the disclosure of which are incorporated by reference herein in their entirety.

### Methods For Producing An AVP

An AVP can be obtained by creating a mutation in the wild-type Av3 polynucleotide sequence; inserting that Av3 variant polynucleotide (AVP) sequence into the appropriate vector; transforming a host organism in such a way that the AVP is expressed; culturing the host organism to generate the desired amount of AVP; and then purifying the AVP from in and/or around host organism. As used herein, the term “wild-type Av3 polypeptide” and the term “native Av3 polypeptide” are used synonymously herein. A wild-type Av3 polypeptide or native Av3 polypeptide have the amino acid sequence as set forth in SEQ ID NO:1. A wild-type Av3 can be obtained by screening a genomic library using primer probes directed to Av3 polynucleotide sequence. Alternatively, wild-type Av3 polynucleotide sequence and/or Av3 variant polynucleotide sequence can be chemically synthesized. For example, a wild-type Av3 polynucleotide sequence and/or Av3 variant polynucleotide sequence can be generated using the oligonucleotide synthesis methods such as the phosphoramidite; triester, phosphite, or H-Phosphonate methods (see Engels, J. W. and Uhlmann, E. (1989), Gene Synthesis [New Synthetic Methods (77)]. Angew. Chem. Int. Ed. Engl., 28: 716-734, the disclosure of which is incorporated herein by reference in its entirety). In some embodiments, the polynucleotide sequence encoding the AVP can be chemically synthesized using commercially available polynucleotide synthesis services such as those offered by Genewiz® (e.g., TurboGENE™; PriorityGENE; and FragmentGENE), or Sigma-Aldrich® (e.g., Custom DNA and RNA Oligos Design and Order Custom DNA Oligos). Exemplary method for generating DNA and or custom chemically synthesized polynucleotides are well known in the art, and are illustratively provided in U.S. Pat. No. 5,736,135, Ser. No. 08/389,615, filed on Feb. 13, 1995, the disclosure of which is incorporated herein by reference in its entirety. See also Agarwal, et al., Chemical synthesis of polynucleotides. Angew Chem Int Ed Engl. 1972 June; 11(6):451-9; Ohtsuka et al., Recent developments in the chemical synthesis of polynucleotides. Nucleic Acids Res. 1982 Nov. 11; 10(21): 6553-6570; Sondek & Shortle. A general strategy for random insertion and substitution mutagenesis: substoichiometric coupling of trinucleotide phosphoramidites. Proc Natl Acad Sci USA. 1992 Apr. 15; 89(8): 3581-3585; Beaucage S. L., et al., Advances in the Synthesis of Oligonucleotides by the Phosphoramidite Approach. Tetrahedron, Elsevier Science Publishers, Amsterdam, NL, vol. 48, No. 12, 1992, pp. 2223-2311; Agrawal (1993) Protocols for Oligonucleotides and Analogs: Synthesis and Properties; Methods in Molecular Biology Vol. 20, the disclosure of which is incorporated herein by reference in its entirety.

Producing a mutation in wild-type Av3 can be achieved by various means that are well known to those having ordinary skill in the art. Methods of mutagenesis include Kunkel's method; cassette mutagenesis; PCR site-directed mutagenesis; the “perfect murder” technique (delitto perfetto); direct gene deletion and site-specific mutagenesis with PCR and one recyclable marker; direct gene deletion and site-specific mutagenesis with PCR and one recyclable marker using long homologous regions; transplacement “pop-in pop-out” method; and CRISPR-Cas 9. Exemplary methods of site-directed mutagenesis can be found in Ruvkun & Ausubel, A general method for site-directed mutagenesis in prokaryotes. Nature. 1981 Jan. 1; 289(5793):85-8; Wallace et al., Oligonucleotide directed mutagenesis of the human beta-globin gene: a general method for producing specific point mutations in cloned DNA. Nucleic Acids Res. 1981 Aug. 11; 9(15):3647-56; Dalbadie-McFarland et al., Oligonucleotide-directed mutagenesis as a general and powerful method for studies of protein function. Proc Natl Acad Sci USA. 1982 November; 79(21):6409-13; Bachman. Site-directed mutagenesis. Methods Enzymol. 2013; 529:241-8; Carey et al., PCR-mediated site-directed mutagenesis. Cold Spring Harb Protoc. 2013 Aug. 1; 2013(8):738-42; and Cong et al., Multiplex genome engineering using CRISPR/Cas systems. Science. 2013 Feb. 15; 339(6121):819-23, the disclosures of all of the aforementioned references are incorporated herein by reference in their entireties.

Chemically synthesizing polynucleotides allows for a DNA sequence to be generated that is tailored to produce a desired polypeptide based on the arrangement of nucleotides within said sequence (i.e., the arrangement of cytosine [C], guanine [G], adenine [A] or thymine [T] molecules); the mRNA sequence that is transcribed from the chemically synthesized DNA polynucleotide can be translated to a sequence of amino acids, each amino acid corresponding to a codon in the mRNA sequence. Accordingly, the amino acid composition of a polypeptide chain that is translated from an mRNA sequence can be altered by changing the underlying codon that determines which of the 20 amino acids will be added to the growing polypeptide; thus, mutations in the DNA such as insertions, substitutions, deletions, and frameshifts may cause amino acid insertions, substitutions, or deletions, depending on the underlying codon.

Obtaining an AVP from a chemically synthesized DNA polynucleotide sequence and/or a wild-type DNA polynucleotide sequence that has been altered via mutagenesis can be achieved by cloning the DNA sequence into an appropriate vector. There are a variety of expression vectors available, host organisms, and cloning strategies known to those having ordinary skill in the art. For example, the vector can be a plasmid, which can introduce a heterologous gene and/or expression cassette into yeast cells to be transcribed and translated. The term “vector” is used to refer to a carrier nucleic acid molecule into which a nucleic acid sequence can be inserted for introduction into a cell where it can be replicated. A vector may contain “vector elements” such as an origin of replication (ORI); a gene that confers antibiotic resistance to allow for selection; multiple cloning sites; a promoter region; a selection marker for non-bacterial transfection; and a primer binding site. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Sambrook et al., 1989 and Ausubel et al., 1996, both incorporated herein by reference. In addition to encoding an Av3 variant polynucleotide, a vector may encode a targeting molecule. A targeting molecule is one that directs the desired nucleic acid to a particular tissue, cell, or other location.

In some embodiments, the cloning strategy Av3 variant polynucleotide can be cloned into a vector using a variety of cloning strategies, and commercial cloning kits and materials readily available to those having ordinary skill in the art. For example, the Av3 variant polynucleotide can be cloned into a vector using such strategies as the SnapFast; Gateway; TOPO; Gibson; LIC; InFusionHD; or Electra strategies. There are numerous commercially available vectors that can be used to produce AVP. For example, an Av3 variant polynucleotide can be generated using polymerase chain reaction (PCR), and combined with a pCR™II_TOPO vector, or a PCR™ 2. 1-TOPO vector (commercially available as the TOPO® TA Cloning® Kit from Invitrogen) for 5 minutes at room temperature; the TOPO® reaction can then be transformed into competent cells, which can subsequently be selected based on color change (see Janke et al., A versatile toolbox for PCR-based tagging of yeast genes: new fluorescent proteins, more markers and promoter substitution cassettes. Yeast. 2004 August; 21(11):947-62; see also, Adams et al. Methods in Yeast Genetics. Cold Spring Harbor, NY, 1997, the disclosure of which is incorporated herein by reference in its entirety).

In some embodiments, a polynucleotide encoding an AVP can be cloned into a vector such as a plasmid, cosmid, virus (bacteriophage, animal viruses, and plant viruses), and/or artificial chromosome (e.g., YACs).

In some embodiments, a polynucleotide encoding an AVP can be inserted into a vector, for example, a plasmid vector using E. coli as a host, by performing the following: digesting about 2 to 5 μg of vector DNA using the restriction enzymes necessary to allow the DNA segment of interest to be inserted, followed by overnight incubation to accomplish complete digestion (alkaline phosphatase may be used to dephosphorylate the 5′-end in order to avoid self-ligation/recircularization); gel purify the digested vector. Next, amplify the DNA segment of interest, for example, a polynucleotide encoding an AVP, via PCR, and remove any excess enzymes, primers, unincorporated dNTPs, short-failed PCR products, and/or salts from the PCR reaction using techniques known to those having ordinary skill in the art (e.g., by using a PCR clean-up kit). Ligate the DNA segment of interest to the vector by creating a mixture comprising: about 20 ng of vector; about 100 to 1,000 ng or DNA segment of interest; 2 μL 10× buffer (i.e., 30 mM Tris-HCl 4 mM MgCl₂, 26 μM NAD, 1 mM DTT, 50 μg/ml BSA, pH 8, stored at 25° C.); 1 μL T4 DNA ligase; all brought to a total volume of 20 μL by adding H₂O. The ligation reaction mixture can then be incubated at room temperature for 2 hours, or at 16° C. for an overnight incubation. The ligation reaction (i.e., about 1 μL) can then be transformed to competent cell, for example, by using electroporation or chemical methods, and a colony PCR can then be performed to identify vectors containing the DNA segment of interest.

In some embodiments a polynucleotide encoding an AVP, along with other DNA segments together composing an AVP expression ORF can be designed for secretion from host yeast cells. An illustrative method of designing an AVP expression ORF is as follows: the ORF can begin with a signal peptide sequence, followed by a DNA sequence encoding a Kex2 cleavage site (Lysine-Arginine), followed by the AVP polynucleotide transgene with addition of glycine-serine codons at the 5′-end, and finally a stop codon at the 3′-end. All these elements will be expressed to a fusion peptide in yeast cells as a single open reading frame (ORF). An α-mating factor (aMF) signal sequence is most frequently used to facilitate metabolic processing of the recombinant insecticidal peptides through the endogenous secretion pathway of the recombinant yeast, i.e. the expressed fusion peptide will typically enter the Endoplasmic Reticulum, wherein the α-mating factor signal sequence is removed by signal peptidase activity, and then the resulting pro-insecticidal peptide will be trafficked to the Golgi Apparatus, in which the Lysine-Arginine dipeptide mentioned above is completely removed by Kex2 endoprotease, after which the mature, polypeptide (i.e., AVP), is secreted out of the cells.

In some embodiments, polypeptide expression levels in recombinant yeast cells can be enhanced by optimizing the codons based on the specific host yeast species. Naturally occurring frequencies of codons observed in endogenous open reading frames of a given host organism need not necessarily be optimized for high efficiency expression. Furthermore, different yeast species (for example, Kluyveromyces lactis, Pichia pastoris, Saccharomyces cerevisiae, etc.) have different optimal codons for high efficiency expression. Hence, codon optimization should be considered for the AVP expression ORF, including the sequence elements encoding the signal sequence, the Kex2 cleavage site and the AVP, since they are initially translated as one fusion peptide in the recombinant yeast cells.

In some embodiments, a codon-optimized AVP expression ORF can be ligated into a yeast-specific expression vectors for yeast expression. There are many expression vectors available for yeast expression, including episomal vectors and integrative vectors, and they are usually designed for specific yeast strains. One should carefully choose the appropriate expression vector in view of the specific yeast expression system which will be used for the peptide production. In some embodiments, integrative vectors can be used, which integrate into chromosomes of the transformed yeast cells and remain stable through cycles of cell division and proliferation. The integrative DNA sequences are homologous to targeted genomic DNA loci in the transformed yeast species, and such integrative sequences include pLAC4, 25S rDNA, pAOX1, and TRP2, etc. The locations of insecticidal peptide transgenes can be adjacent to the integrative DNA sequence (Insertion vectors) or within the integrative DNA sequence (replacement vectors).

In some embodiments, the expression vectors can contain E. coli elements for DNA preparation in E. coli, for example, E. coli replication origin, antibiotic selection marker, etc. In some embodiments, vectors can contain an array of the sequence elements needed for expression of the transgene of interest, for example, transcriptional promoters, terminators, yeast selection markers, integrative DNA sequences homologous to host yeast DNA, etc. There are many suitable yeast promoters available, including natural and engineered promoters, for example, yeast promoters such as pLAC4, pAOX1, pUPP, pADH1, pTEF, pGal1, etc., and others, can be used in some embodiments.

In some embodiments, selection methods such as acetamide prototrophy selection; zeocin-resistance selection; geneticin-resistance selection; nourseothricin-resistance selection; uracil deficiency selection; and/or other selection methods may be used.

In some embodiments, a polynucleotide encoding AVP can be inserted into a pKLAC1 plasmid. The pKLAC1 is commercially available from New England Biolabs® Inc., (item no. (NEB #E1000). The pKLAC1 is designed to accomplish high-level expression of recombinant protein (e.g., AVP) in the yeast Kluyveromyces lactis. The pKLAC1 plasmid can be ordered alone, or as part of a K. lactis Protein Expression Kit. The pKLAC1 plasmid can be linearized using the SacII or BstXI restriction enzymes, and possesses a MCS downstream of an aMF secretion signal. The aMF secretion signal directs recombinant proteins to the secretory pathway, which is then subsequently cleaved via Kex2 resulting in peptide of interest, for example, an AVP. Kex2 is a calcium-dependent serine protease, which is involved in activating proproteins of the secretory pathway, and is commercially available (PeproTech®; item no. 450-45).

In some embodiments, polynucleotide encoding AVP can be inserted into a pLB102 plasmid, or subcloned into a pLB102 plasmid subsequent to selection of yeast colonies transformed with pKLAC1 plasmids ligated with polynucleotide encoding an AVP. Yeast, for example K. lactis, transformed with a pKLAC1 plasmids ligated with polynucleotide encoding a AVP can be selected based on acetamidase (amdS), which allows transformed yeast cells to grow in YCB medium containing acetamide as its only nitrogen source. Once positive yeast colonies transformed with a pKLAC1 plasmids ligated with polynucleotide encoding an AVP are identified.

In addition to the DNA polynucleotide sequence that encodes an AVP, additional DNA segments known as regulatory elements can be cloned into a vector that allow for enhanced expression of the foreign DNA or transgene; examples of such additional DNA segments include (1) promoters and/or enhancer elements; (2) an appropriate mRNA stabilizing polyadenylation signal; (3) an internal ribosome entry site (IRES); (4) introns; and (5) post-transcriptional regulatory elements. The combination of a DNA segment of interest (e.g., avp) with any one of the foregoing cis-acting elements is called an “expression cassette.”

A single expression cassette can contain one or more of the aforementioned regulatory elements, and a polynucleotide operable to express an AVP. For example, in some embodiments, an AVP expression cassette can comprise polynucleotide operable to express an AVP, and an alpha-MF signal; Kex2 site; LAC4 terminator; ADN1 promoter; acetamidase (amdS); flanked by LAC4 promoters on the 5′-end and 3′-end.

In some embodiments, there can be numerous expression cassettes cloned into a vector. For example, in some embodiments, there can be a fSSIt expression cassette comprising a polynucleotide operable to express an AVP. In alternative embodiments, there are two expression cassettes operable to encode an AVP (i.e., a double expression cassette). In other embodiments, there are three expression cassettes operable to encode operable to encode the mutant Av3 polypeptide of the fSSIt expression cassette (i.e., a triple expression cassette).

In some embodiments, a double expression cassette can be generated by subcloning a second AVP expression cassette into a vector containing a fSSIt AVP expression cassette.

In some embodiments, a triple expression cassette can be generated by subcloning a third AVP expression cassette into a vector containing a fSSIt and a second AVP expression cassette.

In some embodiments, a yeast cell transformed with one or more AVP expression cassette s can produce AVP in a yeast culture with a yield of at least: 70 mg/L, 80 mg/L, 90 mg/L, 100 mg/L, 110 mg/L, 120 mg/L, 130 mg/L, 140 mg/L, 150 mg/L, 160 mg/L, 170 mg/L, 180 mg/L, 190 mg/L 200 mg/L, 500 mg/L, 750 mg/L, 1,000 mg/L, 1,250 mg/L, 1,500 mg/L, 1,750 mg/L or at least 2,000 mg/L of AVP per liter of yeast culture medium.

In some embodiments, one or more expression cassettes comprising a polynucleotide operable to express an AVP can be inserted into a vector, for example a pLB103b plasmid, resulting in a yield of about 100 mg/L of AVP (supernatant of yeast fermentation broth). For example, in some embodiments, two expression cassettes comprising a polynucleotide operable to express an AVP can be inserted into a vector, for example a pKS022 plasmid, resulting in a yield of about 2 g/L of AVP (supernatant of yeast fermentation broth). Alternatively, in some embodiments, three expression cassettes comprising a polynucleotide operable to express an AVP can be inserted into a vector, for example a pLB103bT plasmid.

In some embodiments, multiple AVP expression cassettes can be transfected into yeast in order to enable integration of more copies of the optimized AVP transgene into the K. lactis genome. An exemplary method of introducing multiple AVP expression cassettes into a K. lactis genome is as follows: an AVP expression cassette DNA sequence is synthesized, comprising an intact LAC4 promoter element, a codon-optimized AVP expression ORF element and a pLAC4 terminator element; the intact expression cassette is ligated into the pLB103b vector between Sal I and Kpn I restriction sites, downstream of the pLAC4 terminator of pLB10V5, resulting in the double transgene AVP expression vector, pKS022; the double transgene vectors, pKS022, are then linearized using Sac II restriction endonuclease and transformed into YCT306 strain of K. lactis by electroporation, developed in Vestaron; resulting yeast colonies are then grown on YCB agar plate supplemented with 5 mM acetamide, which only the acetamidase-expressing cells could use efficiently as a metabolic source of nitrogen. To evaluate the yeast colonies, about 100 to 400 colonies can be picked from the pKS022 yeast plates. Inoculate from the colonies are each cultured in 2.2 mL of the defined K. lactis media with 2% sugar alcohol added as a carbon source. Cultures are incubated at 23.5° C., with shaking at 280 rpm, for six days, at which point cell densities in the cultures will reach their maximum levels as indicated by light absorbance at 600 nm (OD600). Cells are then removed from the cultures by centrifugation at 4,000 rpm for 10 minutes, and the resulting supernatants (conditioned media) are filtered through 0.2 μM membranes for HPLC yield analysis.

Peptide synthesis or the chemical synthesis or peptides and/or polypeptides can be used to generate AVPs: these methods can be performed by those having ordinary skill in the art, and/or through the use of commercial vendors (e.g., GenScript®; Piscataway, New Jersey). For example, in some embodiments, chemical peptide synthesis can be achieved using Liquid phase peptide synthesis (LPPS), or solid phase peptide synthesis (SPPS). Exemplary methods of peptide synthesis can be found in Anderson G. W. and McGregor A. C. (1957) T-butyloxycarbonylamino acids and their use in peptide synthesis. Journal of the American Chemical Society. 79, 6180-3; Carpino L. A. (1957) Oxidative reactions of hydrazines. Iv. Elimination of nitrogen from 1, 1-disubstituted-2-arenesulfonhydrazidesl-4. Journal of the American Chemical Society. 79, 4427-31; McKay F. C. and Albertson N. F. (1957) New amine-masking groups for peptide synthesis. Journal of the American Chemical Society. 79, 4686-90; Merrifield R. B. (1963) Solid phase peptide synthesis. I. The synthesis of a tetrapeptide. Journal of the American Chemical Society. 85, 2149-54; Carpino L. A. and Han G. Y. (1972) 9-fluorenylmethoxycarbonyl amino-protecting group. The Journal of Organic Chemistry. 37, 3404-9; and A Lloyd-Williams P. et al. (1997) Chemical approaches to the synthesis of peptides and proteins. Boca Raton: CRC Press. 278, the disclosures of which are incorporated herein by reference in their entirety.

In some embodiments, peptide synthesis can generally be achieved by using a strategy wherein the coupling the carboxyl group of a subsequent amino acid to the N-terminus of a preceding amino acid generates the nascent polypeptide chain—a process that is opposite to the type of polypeptide synthesis that occurs in nature.

Peptide deprotection is an important first step in the chemical synthesis of polypeptides. Peptide deprotection is the process in which the reactive groups of amino acids are blocked through the use of chemicals in order to prevent said amino acid's functional group from taking part in an unwanted or non-specific reaction or side reaction; in other words, the amino acids are “protected” from taking part in these undesirable reactions.

Prior to synthesizing the peptide chain, the amino acids must be “deprotected” to allow the chain to form (i.e., amino acids to bind). Chemicals used to protect the N-termini include 9-fluorenylmethoxycarbonyl (Fmoc), and tert-butoxycarbonyl (Boc), each of which can be removed via the use of a mild base (e.g., piperidine) and a moderately strong acid (e.g., trifluoracetic acid (TFA)), respectively.

The C-terminus protectant required is dependent on the type of chemical peptide synthesis strategy used: e.g., LPPS requires protection of the C-terminal amino acid, whereas SPPS does not owing to the solid support which acts as the protecting group. Side chain amino acids require the use of several different protecting groups that vary based on the individual peptide sequence and N-terminal protection strategy; typically, however, the protecting group used for side chain amino acids are based on the tert-butyl (tBu) or benzyl (Bzl) protecting groups.

Amino acid coupling is the next step in a peptide synthesis procedure. To effectuate amino acid coupling, the incoming amino acid's C-terminal carboxylic acid must be activated: this can be accomplished using carbodiimides such as diisopropylcarbodiimide (DIC), or dicyclohexylcarbodiimide (DCC), which react with the incoming amino acid's carboxyl group to form an O-acylisourea intermediate. The O-acylisourea intermediate is subsequently displaced via nucleophilic attack via the primary amino group on the N-terminus of the growing peptide chain. The reactive intermediate generated by carbodiimides can result in the racemization of amino acids. To avoid racemization of the amino acids, reagents such as 1-hydroxybenzotriazole (HOBt) are added in order to react with the O-acylisourea intermediate. Other couple agents that may be used include 2-(1H-benzotriazol-1-yl)-1,1,3,3-tetramethyluronium hexafluorophosphate (HBTU), and benzotriazol-1-yl-oxy-tris(dimethylamino)phosphonium hexafluorophosphate (BOP), with the additional activating bases. Finally, following amino acid deprotection and coupling,

At the end of the synthesis process, removal of the protecting groups from the polypeptide must occur—a process that usually occurs through acidolysis. Determining which reagent is required for peptide cleavage is a function of the protection scheme used and overall synthesis method. For example, in some embodiments, hydrogen bromide (HBr); hydrogen fluoride (HF); or trifluoromethane sulfonic acid (TFMSA) can be used to cleave Bzl and Boc groups. Alternatively, in other embodiments, a less strong acid such as TFA can effectuate acidolysis of tBut and Fmoc groups. Finally, peptides can be purified based on the peptide's physiochemical characteristics (e.g., charge, size, hydrophobicity, etc.). Techniques that can be used to purify peptides include Purification techniques include Reverse-phase chromatography (RPC); Size-exclusion chromatography; Partition chromatography; High-performance liquid chromatography (HPLC); and Ion exchange chromatography (IEC).

### Culture and Transformation Techniques For Producing An AVP

Transformation describes the process of introducing exogenous DNA to a host organism. As used herein, when no organism is specified (i.e., bacteria or eukaryote), then the term “transformation” and “transfection” are used synonymously; otherwise, the term “transformation” refers to the introduction of exogenous DNA to bacteria and yeast, and the term “transfection” refers to the introduction of exogenous DNA to eukaryotic cells (e.g., plants).

In some embodiments, a host cell can be transformed using the following methods: electroporation; cell squeezing; microinjection; impalefection; the use of hydrostatic pressure; sonoporation; optical transfection; continuous infusion; lipofection; through the use of viruses such as adenovirus, adeno-associated virus, lentivirus, herpes simplex virus, and retrovirus; the chemical phosphate method; endocytosis via DEAE-dextran or polyethylenimine (PEI); protoplast fusion; hydrodynamic deliver; magnetofection; nucleoinfection; and/or others. Exemplary methods regarding transfection and/or transformation techniques can be found in Makrides (2003), Gene Transfer and Expression in Mammalian Cells, Elvesier; Wong, TK & Neumann, E. Electric field mediated gene transfer. Biochem. Biophys. Res. Commun. 107, 584-587 (1982); Potter & Heller, Transfection by Electroporation. Curr Protoc Mol Biol. 2003 May; CHAPTER: Unit-9.3; Kim & Eberwine, Mammalian cell transfection: the present and the future. Anal Bioanal Chem. 2010 August; 397(8): 3173-3178, each of these references are incorporated herein by reference in their entireties.

Electroporation is a technique in which electricity is applied to cells causing the cell membrane to become permeable; this in turn allows exogenous DNA to be introduced into the cells. Electroporation is readily known to those having ordinary skill in the art, and the tools and devices required to achieve electroporation are commercially available (e.g., Gene Pulser Xcell™ Electroporation Systems, Bio-Rad®; Neon® Transfection System for Electroporation, Thermo-Fisher Scientific; and other tools and/or devices). Exemplary methods of electroporation are illustrated in Potter & Heller, Transfection by Electroporation. Curr Protoc Mol Biol. 2003 May; CHAPTER: Unit-9.3; Saito (2015) Electroporation Methods in Neuroscience. Springer press; Pakhomov et al., (2017) Advanced Electroporation Techniques in Biology and Medicine. Taylor & Francis; the disclosure of which is incorporated herein by reference in its entirety.

In some embodiments, electroporation can be used to introduce a vector containing a polynucleotide encoding an AVP into yeast, for example, an AVP cloned into a pLB102 plasmid, and transformed into K. lactis cells via electroporation, can be accomplished by inoculating about 10-200 mL of yeast extract peptone dextrose (YEPD) with a suitable yeast species, for example, Kluyveromyces lactis, Saccharomyces cerevisiae, Pichia pastoris, etc., and incubate on a shaker at 30° C. until the early exponential phase of yeast culture (e.g. about 0.6 to 2×10⁸ cells/mL); harvesting the yeast in sterile centrifuge tube and centrifuging at 3000 rpm for 5 minutes at 4° C. (note: keep cells chilled during the procedure) washing cells with 40 mL of ice cold, sterile deionized water, and pelleting the cells a 23,000 rpm for 5 minutes; repeating the wash step, and the resuspending the cells in 20 mL of 1M fermentable sugar, e.g. galactose, maltose, latotriose, sucrose, fructose or glucose and/or sugar alcohol, for example, erythritol, hydrogenated starch hydrolysates, isomalt, lactitol, maltitol, mannitol, and xylitol, followed by spinning down at 3,000 rpm for 5 minutes; resuspending the cells with proper volume of ice cold 1M fermentable sugar, e.g. galactose, maltose, latotriose, sucrose, fructose or glucose and/or a sugar alcohol, for example, erythritol, hydrogenated starch hydrolysates, isomalt, lactitol, maltitol, mannitol, and xylitol to final cell density of 3×10⁹ cell/mL; mixing 40 μl of the yeast suspension with about 1-4 μl of the vector containing a linear polynucleotide encoding an AVP (˜1 μg) in a prechilled 0.2 cm electroporation cuvette (note: ensure the sample is in contact with both sides of the aluminum cuvette); providing a single pulse at 2000 V, for optimal time constant of 5 ms of the RC circuit, the cells was then let recovered in 0.5 ml YED and 0.5 mL 1M fermentable sugar, e.g. galactose, maltose, latotriose, sucrose, fructose or glucose and/or a sugar alcohol, for example, erythritol, hydrogenated starch hydrolysates, isomalt, lactitol, maltitol, mannitol, and xylitol mixture, and then spreading onto selective plates.

In some embodiments, electroporation can be used to introduce a vector containing a polynucleotide encoding an AVP into plant protoplasts by incubating sterile plant material in a protoplast solution (e.g., around 8 mL of 10 mM 2-[N-morpholino]ethanesulfonic acid (MES), pH 5.5; 0.01% (w/v) pectylase; 1% (w/v) macerozyme; 40 mM CaCl₂; and 0.4 M mannitol) and adding the mixture to a rotary shaker for about 3 to 6 hours at 30° C. to produce protoplasts; removing debris via 80-μm-mesh nylon screen filtration; rinsing the screen with about 4 ml plant electroporation buffer (e.g., 5 mM CaCl₂; 0.4 M mannitol; and PBS); combining the protoplasts in a sterile 15 mL conical centrifuge tube, and then centrifuging at about 300×g for about 5 minutes; subsequent to centrifugation, discarding the supernatant and washing with 5 mL of plant electroporation buffer; resuspending the protoplasts in plant electroporation buffer at about 1.5×10⁶ to 2×10⁶ protoplasts per mL of liquid; transferring about 0.5-mL of the protoplast suspension into one or more electroporation cuvettes, set on ice, and adding the vector (note: for stable transformation, the vector should be linearized using anyone of the restriction methods described above, and about 1 to 10 μg of vector may be used; for transient expression, the vector may be retained in its supercoiled state, and about 10 to 40 μg of vector may be used); mixing the vector and protoplast suspension; placing the cuvette into the electroporation apparatus, and shocking for one or more times at about 1 to 2 kV (a 3- to 25-μF capacitance may be used initially while optimizing the reaction); returning the cuvette to ice; diluting the transformed cells 20-fold in complete medium; and harvesting the protoplasts after about 48 hours.

The use of yeast cells as a host organism to generate recombinant AVP is an exceptional method, well known to those having ordinary skill in the art. In some embodiments, the methods and compositions described herein can be performed with any species of yeast, including but not limited to any species of the genus Saccharomyces, Pichia, Kluyveromyces, Hansenula, Yarrowia or Schizosaccharomyces and the species Saccharomyces includes any species of Saccharomyces, for example Saccharomyces cerevisiae species selected from following strains: INVSc1, YNN27, S150-2B, W303-1B, CG25, W3124, JRY188, BJ5464, AH22, GRF18, W303-1A and BJ3505. In some embodiments, members of the Pichia species including any species of Pichia, for example the Pichia species, Pichia pastoris, for example, the Pichia pastoris is selected from following strains: Bg08, Y-11430, X-33, GS115, GS190, JC220, JC254, GS200, JC227, JC300, JC301, JC302, JC303, JC304, JC305, JC306, JC307, JC308, YJN165, KM71, MC100-3, SMD1163, SMD1165, SMD1168, GS241, MS105, any pep4 knock-out strain and any prb1 knock-out strain, as well as Pichia pastoris selected from following strains: Bg08, X-33, SMD1168 and KM71. In some embodiments, any Kluyveromyces species can be used to accomplish the methods described here, including any species of Kluyveromyces, for example, Kluyveromyces lactis, and we teach that the stain of Kluyveromyces lactis can be but is not required to be selected from following strains: GG799, YCT306, YCT284, YCT389, YCT390, YCT569, YCT598, NRRL Y-1140, MW98-8C, MS1, CBS293.91, Y721, MD2/1, PM6-7A, WM37, K6, K7, 22AR1, 22A295-1, SD11, MG1/2, MSK110, JA6, CMK5, HP101, HP108 and PM6-3C, in addition to Kluyveromyces lactis species is selected from GG799, YCT306 and NRRL Y-1140.

In some embodiments, the procedures and methods described here can be accomplished with any species of yeast, including but not limited to any species of Hansenula species including any species of Hansenula and preferably Hansenula polymorpha. In some embodiments, the procedures and methods described here can be accomplished with any species of yeast, including but not limited to any species of Yarrowia species for example, Yarrowia lipolytica. In some embodiments, the procedures and methods described here can be accomplished with any species of yeast, including but not limited to any species of Schizosaccharomyces species including any species of Schizosaccharomyces and preferably Schizosaccharomyces pombe.

In some embodiments, yeast species such as Kluyveromyces lactis, Saccharomyces cerevisiae, Pichia pastoris, and others, can be used as a host organism. Yeast cell culture techniques are well known to those having ordinary skill in the art. Exemplary methods of yeast cell culture can be found in Evans, Yeast Protocols. Springer (1996); Bill, Recombinant Protein Production in Yeast. Springer (2012); Hagan et al., Fission Yeast: A Laboratory Manual, CSH Press (2016); Konishi et al., Improvement of the transformation efficiency of Saccharomyces cerevisiae by altering carbon sources in pre-culture. Biosci Biotechnol Biochem. 2014; 78(6):1090-3; Dymond, Saccharomyces cerevisiae growth media. Methods Enzymol. 2013; 533:191-204; Looke et al., Extraction of genomic DNA from yeasts for PCR-based applications. Biotechniques. 2011 May; 50(5):325-8; and Romanos et al., Culture of yeast for the production of heterologous proteins. Curt Protoc Cell Biol. 2014 Sep. 2; 64:20.9.1-16, the disclosure of which is incorporated herein by reference in its entirety.

Recipes for yeast cell fermentation media and stocks are described as follows: (1) MSM media recipe: 2 g/L sodium citrate dihydrate; 1 g/L calcium sulfate dihydrate (0.79 g/L anhydrous calcium sulfate); 42.9 g/L potassium phosphate monobasic; 5.17 g/L ammonium sulfate; 14.33 g/L potassium sulfate; 11.7 g/L magnesium sulfate heptahydrate; 2 mL/L PTM1trace salt solution; 0.4 ppm biotin (from 500×, 200 ppm stock); 1-2% pure glycerol or other carbon source. (2) PTM1 trace salts solution: Cupric sulfate-5H₂O 6.0 g; Sodium iodide 0.08 g; Manganese sulfate-H₂O 3.0 g; Sodium molybdate-2H₂O 0.2 g; Boric Acid 0.02 g; Cobalt chloride 0.5 g; Zinc chloride 20.0 g; Ferrous sulfate-7H₂O 65.0 g; Biotin 0.2 g; Sulfuric Acid 5.0 ml; add Water to a final volume of 1 liter. An illustrative composition for K. lactis defined medium (DMSor) is as follows: 11.83 g/L KH₂PO₄, 2.299 g/L K₂HPO₄, 20 g/L of a fermentable sugar, e.g., galactose, maltose, latotriose, sucrose, fructose or glucose and/or a sugar alcohol, for example, erythritol, hydrogenated starch hydrolysates, isomalt, lactitol, maltitol, mannitol, and xylitol, 1 g/L MgSO₄·7H₂O, 10 g/L (NH₄)SO₄, 0.33 g/L CaCl₂)·2H₂O, 1 g/L NaCl, 1 g/L KCl, 5 mg/L CuSO₄·5H₂O, 30 mg/L MnSO₄·H₂O, 10 mg/L, ZnCl₂, 1 mg/L KI, 2 mg/L CoCl₂·6H₂O, 8 mg/L Na₂MoO₄·2H₂O, 0.4 mg/L H₃BO₃, 15 mg/L FeCl₃·6H₂O, 0.8 mg/L biotin, 20 mg/L Ca-pantothenate, 15 mg/L thiamine, 16 mg/L myo-inositol, 10 mg/L nicotinic acid, and 4 mg/L pyridoxine.

Yeast cells can be cultured in 48-well Deep-well plates, sealed after inoculation with sterile, air-permeable cover. Colonies of yeast, for example, K. lactis cultured on plates can be picked and inoculated the deep-well plates with 2.2 mL media per well, composed of DMSor. Inoculated deep-well plates can be grown for 6 days at 23.5° C. with 280 rpm shaking in a refrigerated incubator-shaker. On day 6 post-inoculation, conditioned media should be harvested by centrifugation at 4000 rpm for 10 minutes, followed by filtration using filter plate with 0.22 μM membrane, with filtered media are subject to HPLC analyses.

### Yeast Transformation

An exemplary method of yeast transformation is as follows: the expression vectors carrying AVP expression ORF are transformed into yeast cells. FSSIt, the expression vectors are usually linearized by specific restriction enzyme cleavage to facilitate chromosomal integration via homologous recombination. The linear expression vector is then transformed into yeast cells by a chemical or electroporation method of transformation and integrated into the targeted locus of the yeast genome by homologous recombination. The integration can happen at the same chromosomal locus multiple times; therefore, the genome of a transfected yeast cell can contain multiple copies of AVP expression cassette s. The successfully transfected yeast cells can be identified using growth conditions that favor a selective marker engineered into the expression vector and co-integrated into yeast chromosomes with the AVP expression ORF; examples of such markers include, but are not limited to, acetamide prototrophy, zeocin resistance, geneticin resistance, nourseothricin resistance, and uracil prototrophy.

Due to the influence of unpredictable and variable factors-such as epigenetic modification of genes and networks of genes, and variation in the number of integration events that occur in individual cells in a population undergoing a transformation procedure-individual yeast colonies of a given transfection process will differ in their capacities to produce an AVP expression ORF. Therefore, transgenic yeast colonies carrying the AVP transgenes should be screened for high yield strains. Two effective methods for such screening, each dependent on growth of small-scale cultures of the transgenic yeast to provide conditioned media samples for subsequent analysis, use reverse-phase HPLC or housefly injection procedures to analyze conditioned media samples from the positive transgenic yeast colonies.

The transgenic yeast cultures can be performed using 14 mL round bottom polypropylene culture tubes with 5 to 10 mL defined medium added to each tube, or in 48-well deep well culture plates with 2.2 mL defined medium added to each well. The Defined medium, not containing crude proteinaceous extracts or by-products such as yeast extract or peptone, is used for the cultures to reduce the protein background in the conditioned media harvested for the later screening steps. The cultures are performed at the optimal temperature, for example, 23.5° C. for K. lactis, for about 5-6 days, until the maximum cell density is reached. AVPs will now be produced by the transformed yeast cells and secreted out of cells to the growth medium. To prepare samples for the screening, cells are removed from the cultures by centrifugation and the supernatants are collected as the conditioned media, which are then cleaned by filtration through 0.22 μm filter membrane and then made ready for strain screening.

In some embodiments, positive yeast colonies transfected with AVP can be screened via reverse-phase HPLC (rpHPLC) screening of putative yeast colonies. In this screening method, an HPLC analytic column with bonded phase of C18 can be used. Acetonitrile and water are used as mobile phase solvents, and a UV absorbance detector set at 220 nm is used for the peptide detection. Appropriate amounts of the conditioned medium samples are loaded into the rpHPLC system and eluted with a linear gradient of mobile phase solvents. The corresponding peak area of the insecticidal peptide in the HPLC chromatograph is used to quantify the AVP concentrations in the conditioned media. Known amounts of pure AVP are run through the same rpHPLC column with the same HPLC protocol to confirm the retention time of the peptide and to produce a standard peptide HPLC curve for the quantification.

An exemplary reverse-phase HPLC screening process of positive K. lactis cells is as follows: an AVP expression ORF can be inserted into the expression vector, pKLAC1, and transfected into the K. lactis strain, YCT306, from New England Biolabs, Ipswich, MA, USA. pKLAC1 vector is an integrative expression vector. Once the AVP transgenes were cloned into pKLAC1 and transformed into YCT306, their expression was controlled by the LAC4 promoter. The resulting transfected colonies produced pre-propeptides comprising an α-mating factor signal peptide, a Kex2 cleavage site and mature AVPs. The α-Mating factor signal peptide guides the pre-propeptides to enter the endogenous secretion pathway, and mature AVPs are released into the growth media.

In some embodiments, codon optimization for AVP expression can be performed in two rounds, for example, in the fSSIt round, based on some common features of high expression DNA sequences, 33 variants of the AVP expression ORF, expressing an α-Mating factor signal peptide, a Kex2 cleavage site and the AVP, are designed and their expression levels are evaluated in the YCT306 strain of K. lactis, resulting in an initial K. lactis expression algorithm; in a second round of optimization, five more variant AVP expression ORF s can be designed based on the initial K. lactis expression algorithm to further fine-tuned the K. lactis expression algorithm, and identify the best ORF for AVP expression in K. lactis. In some embodiments, the resulting DNA sequence from the foregoing optimization can have an open reading frame encoding an α-mating factor signal peptide, a Kex2 cleavage site and a AVP, which can be cloned into the pKLAC1 vector using Hind III and Not I restriction sites, resulting in Av3 variant expression vectors.

In some embodiments, the yeast, Pichia pastoris, can be transformed with an AVP expression cassette. An exemplary method for transforming P. pastoris is as follows: the vectors, pJUGaKR and pJUZaKR, can be used to transfect the AVP in P. pastoris. The pJUGaKR and pJUZaKR vectors are available from Biogrammatics, Carlsbad, California, USA. Both vectors are integrative vectors and use the uracil phosphoribosyltransferase promoter (pUPP) to enhance the heterologous transgene expression. The only difference between the vectors is that pJUGaKR provides G418 resistance to the host yeast, while pJUZaKR provides Zeocin resistance. PaSSI of complementary oligonucleotides, encoding the AVP are designed and synthesized for subcloning into the two yeast expression vectors. Hybridization reactions are performed by mixing the corresponding complementary oligonucleotides to a final concentration of 20 μM in 30 mM NaCl, 10 mM Tris-Cl (all final concentrations), pH 8, and then incubating at 95° C. for 20 min, followed by a 9-hour incubation starting at 92° C. and ending at 17° C., with 3° C. drops in temperature every 20 min. The hybridization reactions will result in DNA fragments encoding AVP. The two P. pastoris vectors are digested with BsaI-HF restriction enzymes, and the double stranded DNA products of the reactions are then subcloned into the linearized P. pastoris vectors using standard procedures. Following verification of the sequences of the subclones, plasmid aliquots are transfected by electroporation into the P. pastoris strain, Bg08. The resulting transfected yeast, selected based on resistance to Zeocin or G418 conferred by elements engineered into vectors pJUZaKR and pJUGaKR, respectively, can be cultured and screened as described herein.

### Yeast Peptide Yield

In some embodiments, AVP yield can be evaluated using an Agilent 1100 HPLC system equipped with an Onyx monolithic 4.5×100 mm, C18 reverse-phase analytical HPLC column and an auto-injector. An illustrative use of the Agilent 1100 HPLC system equipped with an Onyx monolithic 4.5×100 mm, C18 reverse-phase analytical HPLC column and an auto-injector is as follows: filtered conditioned media samples from transfected K. lactis cells are analyzed using Agilent 1100 HPLC system equipped with an Onyx monolithic 4.5×100 mm, C18 reverse-phase analytical HPLC column and an auto-injector by analyzing HPLC grade water and acetonitrile, both containing 0.1% trifluoroacetic acid, constituting the two mobile phase solvents used for the HPLC analyses; the peak areas of both the AVP are analyzed using HPLC chromatographs, and then used to calculate the peptide concentration in the conditioned media, which can be further normalized to the corresponding final cell densities (as determined by OD600 measurements) as normalized peptide yield.

In some embodiments, positive yeast colonies transfected with AVP can be screened using a housefly injection assay. AVP can paralyze/kill houseflies when injected in measured doses through the body wall of the dorsal thorax. The efficacy of the insecticidal peptide can be defined by the median paralysis/lethal dose of the peptide (PD₅₀/LD₅₀), which causes 50% knock-down ratio or mortality of the injected houseflies respectively. The pure AVP is normally used in the housefly injection assay to generate a standard dose-response curve, from which a PD50/LD₅₀ value can be determined. Using a PD₅₀/LD₅₀ value from the analysis of a standard dose-response curve of the pure AVP, quantification of the insecticidal peptide produced by the transfected yeast can be achieved using a housefly injection assay performed with serial dilutions of the corresponding conditioned media.

An exemplary housefly injection bioassay is as follows: conditioned media is serially diluted to generate full dose-response curves from the housefly injection bioassay. Before injection, adult houseflies (Musca domestica) are immobilized with CO₂, and 12-18 mg houseflies are selected for injection. A microapplicator, loaded with a 1 cc syringe and 30-gauge needle, is used to inject 0.5 μL per fly, doses of serially diluted conditioned media samples into houseflies through the body wall of the dorsal thorax. The injected houseflies are placed into closed containers with moist filter paper and breathing holes on the lids, and they are examined by knock-down ratio or by mortality scoring at 24 hours post-injection. Normalized yields are calculated. Peptide yield means the peptide concentration in the conditioned media in units of mg/L. However, peptide yields are not always sufficient to accurately compare the strain production rate. Individual strains may have different growth rates, hence when a culture is harvested, different cultures may vary in cell density. A culture with a high cell density may produce a higher concentration of the peptide in the media, even though the peptide production rate of the strain is lower than another strain which has a higher production rate. Accordingly, the term “normalized yield” is created by dividing the peptide yield with the cell density in the corresponding culture and this allows a better comparison of the peptide production rate between strains. The cell density is represented by the light absorbance at 600 nm with a unit of “A” (Absorbance unit).

Screening yeast colonies that have undergone a transformation with AVP can identify the high yield yeast strains from hundreds of potential colonies. These strains can be fermented in bioreactor to achieve at least up to 4 g/L or at least up to 3 g/L or at least up to 2 g/L yield of the AVP when using optimized fermentation media and fermentation conditions described herein. The higher rates of production (expressed in mg/L) can be anywhere from about 100 mg/L to about 4,000 mg/L, or from about 100 to about 3,000 mg/L, or 100 to 2,000 mg/L, or 100 to 1,500 mg/L, or 100 to 1,000 mg/L, or 100 to 750 mg/L, or 100 to 500 mg/L, or 150 to 4,000 mg/L, or 200 to 4,000 mg/L, or 300 to 4,000 mg/L, or 400 to 4,000 mg/L, or 500 to 4,000 mg/L, or 750 to 4,000 mg/L, or 1,000 to 4,000 mg/L, or 1,250 to 4,000 mg/L, or 1,500 to 4,000 mg/L, or 2,000 to 4,000 mg/L, or 2,500 to 4,000 mg/L, or 3,000 to 4,000 mg/L, or 3,500 to 4,000 mg/L mg/L, or any range of any value provided or even greater yields than can be achieved with a peptide before conversion, using the same or similar production methods that were used to produce the peptide before conversion.

### Compositions and Formulations

Examples of the three AVPs described herein, include the AVPs: (1) Av3a, (2) Av3-C1, and (3) Av3b polypeptides and genes, and include all of the peptides and their coding genes as described in the references provided above and herein. Specific examples of AVP and polypeptides disclosed for purposes of providing examples and not intended to be limiting in any way, are the peptides and their homologies as described above, and in particular peptides and nucleotides which are modified from the Av3 polypeptide originating from the sea anemone, Anemonia viridis, (see SEQ ID NO:1 [NCBI Accession No. P01535.1]). The AVP Av3a, has an amino acid sequence reflecting an N-terminal mutation replacing the amino terminal arginine (R) amino acid with a lysine (K) amino acid (RIK) relative to SEQ ID NO:1. The Av3-C1 polypeptide has a deletion of the C-terminal valine (v) amino acid, relative to SEQ ID NO:1. The third AVP, Av3b is an AVP polypeptide with two mutations, an N-terminal mutation replacing the amino terminal arginine (R) amino acid with a lysine (K) amino acid (R1K) relative to SEQ ID NO:1, and a deletion of the C-terminal valine (v) amino acid, relative to SEQ ID NO:1.

Described and incorporated by reference to the peptides identified herein are homologous variants of sequences mentioned, having homology to such sequences or referred to herein, which are also identified and claimed as suitable for making special according to the processes described herein, including all homologous sequences having at least any of the following percent identities to any of the sequences disclosed here or to any sequence incorporated by reference: 50%, 55%, 60%, 65%, 70%, 75%, 80%, 85%, 90%, 95%, 96%, 97%, 98%, or 99% or greater identity or 100% identity to any and all sequences identified in the sequences noted above, and to any other sequence identified herein, including each and every sequence in the sequence listing of this application. When the term homologous or homology is used herein with a number such as 50% or greater, then what is meant is percent identity or percent similarity between the two peptides. When homologous or homology is used without a numeric percent then it refers to two peptide sequences that are closely related in the evolutionary or developmental aspect in that they share common physical and functional aspects, like topical toxicity and similar size (i.e., the homolog being within 100% greater length or 50% shorter length of the peptide specifically mentioned herein or identified by reference herein as above).

### Exemplary AVP Compositions and Combinations

### Sprayable Compositions

Examples of spray products of the present invention can include field sprayable formulations for agricultural usage and indoor sprays for use in interior spaces in a residential or commercial space. In some embodiments, residual sprays or space sprays comprising an AVP or an insecticidal protein comprising one or more AVPs can be used to reduce or eliminate insect pests in an interior space. Surface spraying indoors (SSI) is the technique of applying a variable volume sprayable volume of an insecticide onto indoor surfaces where vectors rest, such as on walls, windows, floors and ceilings. The primary goal of variable volume sprayable volume is to reduce the lifespan of the insect pest, (for example, a fly, a flea, a tick, or a mosquito vector) and thereby reduce or interrupt disease transmission. The secondary impact is to reduce the density of insect pests within the treatment area. SSI can be used as a method for the control of insect pest vector diseases, such as Lyme disease, Salmonella, Chikungunya virus, Zika virus, and malaria, and can also be used in the management of parasites carried by insect vectors, such as Leishmaniasis and Chagas disease. Many mosquito vectors that harbor Zika virus, Chikungunya virus, and malaria include endophilic mosquito vectors, resting inside houses after taking a blood meal. These mosquitoes are particularly susceptible to control through surface spraying indoors (SSI) with a sprayable composition comprising an AVP or an insecticidal protein comprising one or more AVPs. As its name implies, SSI involves applying the composition onto the walls and other surfaces of a house with a residual insecticide. In one embodiment, the composition containing an AVP or insecticidal protein comprising one or more AVPs and one or more non-AVP peptides, polypeptides and proteins will knock down insect pests that come in contact with these surfaces. SSI does not directly prevent people from being bitten by mosquitoes. Rather, it usually controls insect pests after they have blood fed, if they come to rest on the sprayed surface. SSI thus prevents transmission of infection to other persons. To be effective, SSI must be applied to a very high proportion of households in an area (usually greater than 40-80 percent). Therefore, sprays in accordance with the invention having good residual efficacy and acceptable odor are particularly suited as a component of integrated insect pest vector management or control solutions.

In contrast to SSI, which requires that the active an AVP or an insecticidal protein comprising one or more AVPs is bound to surfaces of dwellings, such as walls, ceiling as with a paint, for example, space spray products of the invention rely on the production of a large number of small insecticidal droplets intended to be distributed through a volume of air over a given period of time. When these droplets impact on a target insect pest, they deliver a knockdown effective dose of the AVP or insecticidal protein comprising one or more AVPs effective to control the insect pest. The traditional methods for generating a space-spray include thermal fogging (whereby a dense cloud of an AVP composition comprising droplets is produced giving the appearance of a thick fog) and Ultra Low Volume (ULV), whereby droplets are produced by a cold, mechanical aerosol-generating machine. Ready-to-use aerosols such as aerosol cans may also be used.

Since large areas can be treated at any one time this method is a very effective way to rapidly reduce the population of flying insect pests in a specific area. Since there is very limited residual activity from the application it must be repeated at intervals of 5-7 days in order to be fully effective. This method can be particularly effective in epidemic situations where rapid reduction in insect pest numbers is required. As such, it can be used in urban dengue control campaigns.

Effective space-spraying is generally dependent upon the following specific principles. Target insects are usually flying through the spray cloud (or are sometimes impacted whilst resting on exposed surfaces). The efficiency of contact between the spray droplets and target insects is therefore crucial. This is achieved by ensuring that spray droplets remain airborne for the optimum period of time and that they contain the right dose of insecticide. These two issues are largely addressed through optimizing the droplet size. If droplets are too big they drop to the ground too quickly and don't penetrate vegetation or other obstacles encountered during application (limiting the effective area of application). If one of these big droplets impacts an individual insect then it is also ‘overkill’ since a high dose will be delivered per individual insect. If droplets are too small then they may either not deposit on a target insect (no impaction) due to aerodynamics or they can be carried upwards into the atmosphere by convection currents. The optimum size of droplets for space-spray application are droplets with a Volume Median Diameter (VMD) of 10-25 microns.

The active compositions of the present invention comprising at least one AVP or an insecticidal protein comprising one or more AVPs may be made available in a spray product as an aerosol-based application, including aerosolized foam applications. Pressurized cans are the typical vehicle for the formation of aerosols. An aerosol propellant that is compatible with the AVP or an insecticidal protein comprising one or more AVPs is used. Preferably, a liquefied-gas type propellant is used.

Suitable propellants include compressed air, carbon dioxide, butane and nitrogen. The concentration of the propellant in the active compound composition is from about 5 percent to about 40 percent by weight of the pyridine composition, preferably from about 15 percent to about 30 percent by weight of the AVP or an insecticidal protein comprising one or more AVPs containing composition.

In one embodiment, the AVP or insecticidal protein comprising one or more AVPs containing formulations of the invention can also include one or more foaming agents. Foaming agents that can be used include sodium laureth sulphate, cocamide DEA, and cocamidopropyl betaine. Preferably, the sodium laureth sulphate, cocamide DEA and cocamidopropyl are used in combination. The concentration of the foaming agent(s) in the active compound composition is from about 10 percent to about 25 percent by weight, more preferably 15 percent to 20 percent by weight of the composition.

When such formulations are used in an aerosol application not containing foaming agents, the active compositions of the present invention can be used without the need for mixing directly prior to use. However, aerosol formulations containing the foaming agents do require mixing (i.e., shaking) immediately prior to use. In addition, if the formulations containing foaming agents are used for an extended time, they may require additional mixing at periodic intervals during use.

In some embodiments, a dwelling area may also be treated with an active AVP or an insecticidal protein comprising one or more AVPs composition of the present invention by using a burning formulation, such as a candle, a smoke coil or a piece of incense containing the composition. For example, composition may be comprised in household products such as “heated” air fresheners in which insecticidal compositions are released upon heating, for example, electrically, or by burning. The active compound compositions of the present invention containing an AVP or an insecticidal protein comprising one or more AVPs may be made available in a spray product as an aerosol, a mosquito coil, and/or a vaporizer or fogger.

In some embodiments, fabrics and garments may be made containing a pesticidal effective composition comprising an AVP or an insecticidal protein of the present disclosure. In some embodiments, the concentration of the AVP or insecticidal protein comprising one or more AVPs in the polymeric material, fiber, yarn, weave, net, or substrate described herein, can be varied within a relatively wide concentration range from, for example 0.05 to 15 percent by weight, preferably 0.2 to 10 percent by weight, more preferably 0.4 to 8 percent by weight, especially 0.5 to 5, such as 1 to 3, percent by weight.

Similarly, the concentration of the an AVP or an insecticidal protein comprising one or more AVPs in the composition of the invention (whether for treating surfaces or for coating a fiber, yarn, net, weave) can be varied within a relatively wide concentration range from, for example 0.1 to 70 percent by weight, such as 0.5 to 50 percent by weight, preferably 1 to 40 percent by weight, more preferably 5 to 30 percent by weight, especially 10 to 20 percent by weight.

The concentration of the AVP or insecticidal protein comprising one or more AVPs may be chosen according to the field of application such that the requirements concerning knockdown efficacy, durability and toxicity are met. Adapting the properties of the material can also be accomplished and so custom-tailored textile fabrics are obtainable in this way.

Accordingly an effective amount of an AVP or an insecticidal protein comprising one or more AVPs can depend on the specific use pattern, the insect pest against which control is most desired and the environment in which an AVP or an insecticidal protein comprising one or more AVPs will be used. Therefore, an effective amount of an AVP or an insecticidal protein comprising one or more AVPs is sufficient that control of an insect pest is achieved.

In some embodiments, the present disclosure provides compositions or formulations for coating walls, floors and ceilings inside of buildings and for coating a substrate or non-living material, which comprise an AVP or an insecticidal protein comprising one or more AVPs. The inventive compositions can be prepared using known techniques for the purpose in mind, which could contain a binder to facilitate the binding of the compound to the surface or other substrate. Agents useful for binding are known in the art and tend to be polymeric in form. The type of binder suitable for composition to be applied to a wall surface having particular porosities, binding characteristics would be different to a fiber, yarn, weave or net—a skilled person, based on known teachings, would select a suitable binder.

Typical binders are poly vinyl alcohol, modified starch, poly vinyl acrylate, polyacrylic, polyvinyl acetate co polymer, polyurethane, and modified vegetable oils. Suitable binders can include latex dispersions derived from a wide variety of polymers and co-polymers and combinations thereof. Suitable latexes for use as binders in the inventive compositions comprise polymers and copolymers of styrene, alkyl styrenes, isoprene, butadiene, acrylonitrile lower alkyl acrylates, vinyl chloride, vinylidene chloride, vinyl esters of lower carboxylic acids and alpha, beta-ethylenically unsaturated carboxylic acids, including polymers containing three or more different monomer species copolymerized therein, as well as post-dispersed suspensions of silicones or polyurethanes. Also suitable may be a polytetrafluoroethylene (PTFE) polymer for binding the active ingredient to other surfaces.

In some exemplary embodiments, an insecticidal formulation according to the present disclosure may comprises at least one AVP, or insecticidal protein comprising one or more AVPs, (optionally with a secondary invertebrate pest control agent described herein) and a an excipient, diluent or carrier, such as water, and optionally a polymeric binder and optionally further components such as a dispersing agent, a polymerizing agent, an emulsifying agent, a thickener, an alcohol, a fragrance or any other inert excipients used in the preparation of sprayable insecticides known in the art.

The polymeric binder binds the pyridine compounds to the surface of the non-living material and ensures a long-term effect. Using the binder reduces the elimination of the pyridine pesticide out of the non-living material due to environmental effects such as rain or due to human impact on the non-living material such as washing and/or cleaning it. The further components can be an additional insecticide compound, a synergist, a UV stabilizer.

The inventive compositions can be in a number of different forms or formulation types, such as suspensions, capsules suspensions, and a person skilled in the art can prepare the relevant composition based on the properties of the particular AVP, or insecticidal protein comprising one or more AVPs, its uses and also application type. For example, the AVP, or insecticidal protein comprising one or more AVPs used in the methods, embodiments and other aspects of the present disclosure may be encapsulated in the formulation. An encapsulated AVP, or insecticidal protein comprising one or more AVPs can provide improved wash-fastness and also longer period of activity. The formulation can be organic based or aqueous based, preferably aqueous based.

Microencapsulated AVP, or insecticidal protein comprising one or more AVPs suitable for use in the compositions and methods according to the present disclosure may be prepared with any suitable technique known in the art. For example, various processes for microencapsulating material have been previously developed. These processes can be divided into three categories-physical methods, phase separation and interfacial reaction. In the physical methods category, microcapsule wall material and core particles are physically brought together and the wall material flows around the core particle to form the microcapsule. In the phase separation category, microcapsules are formed by emulsifying or dispersing the core material in an immiscible continuous phase in which the wall material is dissolved and caused to physically separate from the continuous phase, such as by coacervation, and deposit around the core particles. In the interfacial reaction category, microcapsules are formed by emulsifying or dispersing the core material in an immiscible continuous phase and then an interfacial polymerization reaction is caused to take place at the surface of the core particles. The concentration of the AVP, or insecticidal protein comprising one or more AVPs present in the microcapsules can vary from 0.1 to 60% by weight of the microcapsule.

The formulation used in the AVP, or insecticidal protein comprising one or more AVPs containing compositions, methods, embodiments and other aspects according to the present disclosure may be formed by mixing all ingredients together with water optionally using suitable mixing and/or dispersing aggregates. In general, such a formulation is formed at a temperature of from 10 to 70° C., preferably 15 to 50° C., more preferably 20 to 40° C. In general, it is possible to use an AVP or an insecticidal protein comprising one or more AVPs (as pesticide) (A), solid polymer (B) and optionally additional additives (D) and to disperse them in the aqueous component (C) If a binder is present in a composition of the present invention, it is preferred to use dispersions of the polymeric binder (B) in water as well as aqueous formulations of the AVP, or insecticidal protein comprising one or more AVPs (A) in water which have been separately prepared before. Such separate formulations may contain additional additives for stabilizing (A) and/or (B) in the respective formulations and are commercially available. In a second process step, such raw formulations and optionally additional water (component (C)) are added. Also combinations are possible, i.e., using a pre-formed dispersion of (A) and/or (B) and mixing it with solid (A) and/or (B). A dispersion of the polymeric binder (B) may be a pre-manufactured dispersion already made by a chemicals manufacturer.

However, it is also within the scope of the present invention to use “hand-made” dispersions, i.e., dispersions made in small-scale by an end-user. Such dispersions may be made by providing a mixture of about 20 percent of the binder (B) in water, heating the mixture to temperature of 90° C. to 100° C. and intensively stirring the mixture for several hours. It is possible to manufacture the formulation as a final product so that it can be readily used by the end-user for the process according to the present invention. However, it is of course also possible to manufacture a concentrate, which may be diluted by the end-user with additional water (C) to the desired concentration for use.

In an embodiment, a composition suitable for SSI application or a coating formulation containing an AVP or an insecticidal protein comprising one or more AVPs contains the active ingredient and a carrier, such as water, and may also one or more co-formulants selected from a dispersant, a wetter, an anti-freeze, a thickener, a preservative, an emulsifier and a binder or sticker.

In some embodiments, an exemplary solid formulation of an AVP or an insecticidal protein comprising one or more AVPs, is generally milled to a desired particle size, such as the particle size distribution d(0.5) is generally from 3 to 20, preferably 5 to 15, especially 7 to 12, μm.

Furthermore, it may be possible to ship the formulation to the end-user as a kit comprising at least a first component comprising an AVP or an insecticidal protein comprising one or more AVPs (A); and a second component comprising at least one polymeric binder (B). Further additives (D) may be a third separate component of the kit, or may be already mixed with components (A) and/or (B). The end-user may prepare the formulation for use by just adding water (C) to the components of the kit and mixing. The components of the kit may also be formulations in water. Of course it is possible to combine an aqueous formulation of one of the components with a dry formulation of the other component(s). As an example, the kit can comprise one formulation of an AVP or an insecticidal protein comprising one or more AVPs (A) and optionally water (C); and a second, separate formulation of at least one polymeric binder (B), water as component (C) and optionally components (D).

The concentrations of the components (A), (B), (C) and optionally (D) will be selected by the skilled artisan depending of the technique to be used for coating/treating. In general, the amount of an AVP or an insecticidal protein comprising one or more AVPs (A) may be up to 50, preferably 1 to 50, such as 10 to 40, especially 15 to 30, percent by weight, based on weight of the composition. The amount of polymeric binder (B) may be in the range of 0.01 to 30, preferably 0.5 to 15, more preferably 1 to 10, especially 1 to 5, percent by weight, based on weight of the composition. If present, in general the amount of additional components (D) is from 0.1 to 20, preferably 0.5 to 15, percent by weight, based on weight of the composition. If present, suitable amounts of pigments and/or dyestuffs and/or fragrances are in general 0.01 to 5, preferably 0.1 to 3, more preferably 0.2 to 2, percent by weight, based on weight of the composition. A typical formulation ready for use comprises 0.1 to 40, preferably 1 to 30, percent of components (A), (B), and optionally (D), the residual amount being water (C). A typical concentration of a concentrate to be diluted by the end-user may comprise 5 to 70, preferably 10 to 60, percent of components (A), (B), and optionally (D), the residual amount being water (C).

### Combination Compositions

One embodiment of an exemplary AVP composition can include a composition comprising a combination of one or more AVPs or one or more insecticidal protein for mixing with a secondary invertebrate pest control agent (SIPCA) that are known insecticides and/or acaricides, which may include one or more of the following SIPCAs selected from: sodium channel modulators such as bifenthrin, cypermethrin, cyhalothrin, lambda-cyhalothrin, cyfluthrin, beta-cyfluthrin, deltamethrin, dimefluthrin, esfenvalerate, fenvalerate, indoxacarb, metofluthrin, profluthrin, pyrethrin and tralomethrin; cholinesterase inhibitors such as chlorpyrifos, methomyl, oxamyl, thiodicarb and triazamate; neonicotinoids such as acetamiprid, clothianidin, dinotefuran, imidacloprid, nitenpyram, nithiazine, thiacloprid and thiamethoxam; insecticidal macrocyclic lactones such as spinetoram, spinosad, abamectin, avermectin and emamectin; GABA (gamma.-aminobutyric acid)-regulated chloride channel blockers such as endosulfan, ethiprole and fipronil; chitin synthesis inhibitors such as buprofezin, cyromazine, flufenoxuron, hexaflumuron, lufenuron, novaluron, noviflumuron and triflumuron; juvenile hormone mimics such as diofenolan, fenoxycarb, methoprene and pyriproxyfen; octopamine receptor ligands such as amitraz; ecdysone agonists such as azadirachtin, methoxyfenozide and tebufenozide; ryanodine receptor ligands such as ryanodine, anthranilic diamides such as chlorantraniliprole and flubendiamide; nereistoxin analogs such as cartap; mitochondrial electron transport inhibitors such as chlorfenapyr, hydramethylnon and pyridaben; lipid biosynthesis inhibitors such as spirodiclofen and spiromesifen; cyclodiene insecticides such as dieldrin; cyflumetofen; fenothiocarb; flonicamid; metaflumizone; pyrafluprole; pyridalyl; pyriprole; pymetrozine; spirotetramat; and thiosultap-sodium. One embodiment of an exemplary SIPCA for mixing with an AVP or insecticidal protein comprising an AVP of this invention can include nucleopolyhedrovirus such as HzNPV and AfNPV; Bacillus thuringiensis and encapsulated delta-endotoxins of Bacillus thuringiensis such as Cellcap, MPV and MPVII; as well as naturally occurring and genetically modified viral insecticides including members of the family Baculoviridae as well as entomophagous fungi.

In some embodiments, an AVP & SIPCA containing combination (an AVP or an insecticidal protein comprising at least one AVP) composition may include one or more SIPCAs selected from: abamectin, acephate, acetamiprid, acetoprole, aldicarb, amidoflumet, amitraz, avermectin, azadirachtin, azinphos-methyl, bifenthrin, bifenazate, bistrifluoron, buprofezin, carbofuran, cartap, chinomethionat, chlorfenapyr, chlorfluazuron, chlo-rantraniliprole, chlorpyrifos, chlorpyrifos-methyl, chlo-robenzilate, chromafenozide, clothianidin, cyflumetofen, cyfluthrin, beta-cyfluthrin, cyhalothrin, gamma-cyhalothrin, lambda-cyhalothrin, cyhexatin, cypermethrin, cyromazine, deltamethrin, diafenthiuron, diazinon, dicofol, dieldrin, dienochlor, diflubenzuron, dimefluthrin, dimethoate, dinote-furan, diofenolan, emamectin, endosulfan, esfenvalerate, ethiprole, etoxazole, fenamiphos, fenazaquin, fenbutatin oxide, fenothiocarb, fenoxycarb, fenpropathrin, fenpyroxi-mate, fenvalerate, fipronil, flonicamid, flubendiamide, flucythrinate, tau-fluvalinate, flufenerim, flufenoxuron, fono-phos, halofenozide, hexaflumuron, hexythiazox, hydrameth-ylnon, imicyafos, imidacloprid, indoxacarb, isofenphos, lufenuron, malathion, metaflumizone, metaldehyde, metha-midophos, methidathion, methomyl, methoprene, methoxy-chlor, methoxyfenozide, metofluthrin, monocrotophos, nitenpyram, nithiazine, novaluron, noviflumuron, oxamyl, parathion, parathion-methyl, permethrin, phorate, phosalone, phosmet, phosphamidon, pirimicarb, profenofos, profluthrin, propargite, protrifenbute, pymetrozine, pyrafluprole, pyre-thrin, pyridaben, pyridalyl, pyrifluquinazon, pyriprole, pyriproxyfen, rotenone, ryanodine, spinetoram, spinosad, spiridiclofen, spiromesifen, spirotetramat, sulprofos, tebufenozide, tebufenpyrad, teflubenzuron, tefluthrin, terbu-fos, tetrachlorvinphos, thiacloprid, thiamethoxam, thiodi-carb, thiosultap-sodium, tolfenpyrad, tralomethrin, triaz-amate, trichlorfon, triflumuron, Bacillus thuringiensis subsp. aizawai, Bacillus thuringiensis subsp. kurstaki, nucleopoly-hedrovirus, an encapsulated delta-endotoxin of Bacillus thuringiensis, baculovirus, entomopathogenic bacteria, ento-mopathogenic virus and entomopathogenic fungi.

Of note is an exemplary combination composition of the present disclosure wherein the combination comprises an AVP or an insecticidal protein comprising at least one AVP in combination with at least one SIPCA selected from a Bacillus thuringiensis biological agent known in the art as an insecticide and/or acaricide. Also of note is an exemplary combination composition of the present disclosure wherein the combination comprises an AVP or an insecticidal protein comprising at least one AVP in combination with at least one SIPCA selected from the group consisting of cypermethrin, cyhalothrin, cyfluthrin, beta-cyfluthrin, esfenvalerate, fenvalerate, tralomethrin, fenothiocarb, methomyl, oxamyl, thiodicarb, acetamiprid, clothianidin, imidacloprid, thiamethoxam, thiacloprid, indoxacarb, spinosad, abamectin, avermectin, emamectin, endosulfan, ethiprole, fipronil, flufenoxuron, triflumuron, diofenolan, pyriproxyfen, pymetrozine, amitraz, Bacillus thuringiensis aisawai, Bacillus thuringiensis kurstaki, Bacillus thuringiensis delta endotoxin and entomophagous fungi.

The weight ratios of a combination composition comprising an AVP or an insecticidal protein comprising at least one AVP in combination with at least one SIPCA, typically are between 1000:1 and 1:1000, with one embodiment being between 500:1 and 1:500, another embodiment being between 250:1 and 1:200 and another embodiment being between 100:1 and 1:50.

In some embodiments, a combination composition comprising an AVP or an insecticidal protein comprising at least one AVP in combination with a biologically effective amount of at least one SIPCA having a similar spectrum of control but a different site of action. In some embodiments, contacting a plant genetically modified to express a SIPCA protein (e.g., a Bt protein) or the locus of the plant with a biologically effective amount of an AVP of this invention can also provide a broader spectrum of plant protection and be advantageous for resistance management.

Table 1 lists specific combinations of an AVP or an insecticidal protein comprising at least one AVP in combination with one or more SIPCAs illustrative of the mixtures, combination compositions and methods of the present disclosure. The First column of Table 1 lists the specific SIPCA (e.g., “Abamectin” in the First line). The second column of Table 1 lists the mode of action (if known) or chemical class of the SIPCA. The third column of Table 1 lists embodiment(s) of ranges of weight ratios for rates at which the SIPCA can be applied relative to an AVP polypeptide or an insecticidal protein comprising at least one AVP, (e.g., “50:1 to 1:50” of abamectin relative to an AVP by weight). Thus, for example, the first line of Table 1 specifically discloses the combination of an AVP with the SIPCA abamectin can be applied in a weight ratio between 50:1 to 1:50. The remaining lines of Table 1 are to be construed similarly. Of further note, Table 1 lists specific combinations of an AVP or an insecticidal protein comprising at least one AVP with other SIPCAs illustrative of the mixtures, combination compositions and methods of the present disclosure and includes additional embodiments of weight ratio ranges for application rates.

TABLE 1
Exemplary Combination Mixtures of an AVP or an
insecticidal protein comprising an AVP and a secondary
invertebrate pest control agent (SIPCA).

| Secondary | | |
|---|---|---|
| Invertebrate Pest | Mode of | Typical |
| Control Agent | Action or Chemical Class | Weight Ratio |
| Abamectin | Macrocyclic lactones | 50:1 to 1:50 |
| Acetamiprid | Neonicotinoids | 150:1 to 1:200 |
| Arnitraz | Octopamine receptor ligands | 200:1 to 1:100 |
| Avermectin | Macrocyclic lactones | 50:1 to 1:50 |
| Azadirachtin | Ecdysone agonists | 100:1 to 1:120 |
| Beta-cyfluthrin | Sodium channel modulators | 150:1 to 1:200 |
| Bifenthrin | Sodium channel modulators | 100:1 to 1:10 |
| Buprofezin | Chitin synthesis inhibitors | 500:1 to 1:50 |
| Cartap | Nereistoxin analogs | 100:1 to 1:200 |
| Chlorantraniliprole | Ryanodine receptor ligands | 100:1 to 1:120 |
| Chlorfenapyr | Mitochondrial electron | 300:1 to 1:200 |
| | transport inhibitors | |
| Chlorpyrifos | Cholinesterase inhibitors | 500:1 to 1:200 |
| Clothianidin | Neonicotinoids | 100:1 to 1:400 |
| Cythathrin | Sodium channel modulators | 150:1 to 1:220 |
| Cyhalothrin | Sodium channel modulators | 150:1 to 1:200 |
| Cypermethrin | Sodium channel modulators | 150:1 to 1:200 |
| Cyromazine | Chitin synthesis inhibitors | 400:1 to 1:50 |
| Deltamethrin | Sodium channel modulators | 50:1 to 1:400 |
| Dieldrin | Cyclodiene insecticides | 200:1 to 1:100 |
| Dinotefuran | Neonicotinoids | 150:1 to 1:200 |
| Diofenolan | Molting inhibitor | 150:1 to 1:200 |
| Emamectin | Macrocyclic lactones | 50:1 to 1:10 |
| Endosulfan | Cyclodiene insecticides | 200:1 to 1:100 |
| Esfenvalerate | Sodium channel modulators | 100:1 to 1:400 |
| Ethiprole | GABA-regulated chloride | 200:1 to 1:100 |
| | channel blockers | |
| Fenothiocarb | Non-systemic | 150:1 to 1:200 |
| Fenoxycarb | Juvenile hormone mimics | 500:1 to 1:100 |
| Fenvalerate | Sodium channel modulators | 150:1 to 1:200 |
| Fipronil | GABA-regulated chloride | 150:1 to 1:100 |
| | channel blockers | |
| Flonicamid | Chordotonal disruptor | 200:1 to 1:100 |
| Flubendiamide | Ryanodine receptor ligands | 100:1 to 1:120 |
| Flufenoxuron | Chitin synthesis inhibitors | 200:1 to 1:100 |
| Hexaflumuron | Chitin synthesis inhibitors | 300:1 to 1:50 |
| Hydramethylnon | Mitochondrial electron | 150:1 to 1:250 |
| | transport inhibitors | |
| Imidacloprid | Neonicotinoids | 1000:1 to 1:1000 |
| Indoxacarb | Sodium channel modulators | 200:1 to 1:50 |
| Lambda-cyhalothrin | Sodium channel modulators | 50:1 to 1:250 |
| Lufenuron | Chitin synthesis inhibitors | 500:1 to 1:250 |
| Metaflumizone | Sodium channel modulators | 200:1 to 1:200 |
| Methomyl | Cholinesterase inhibitors | 500:1 to 1:100 |
| Methoprene | Juvenile hormone mimics | 500:1 to 1:100 |
| Methoxyfenozied | Ecdysone agonists | 50:1 to 1:50 |
| Nitenpyram | Neonicotinoids | 150:1 to 1:200 |
| Nithiazine | Neonicotinoids | 150:1 to 1:200 |
| Novaluron | Chitin synthesis inhibitors | 500:1 to 1:150 |
| Oxamyl | Cholinesterase inhibitors | 200:1 to 1:200 |
| Pymetrozine | Feeding inhibition | 200:1 to 1:100 |
| Pyrethrin | Sodium channel modulators | 100:1 to 1:10 |
| Pyridaben | Mitochondrial electron | 200:1 to 1:100 |
| | transport inhibitors | |
| Pyridalyl | Protein synthesis inhibitor | 200:1 to 1:100 |
| Pyriproxyfen | Juvenile hormone mimics | 500:1 to 1:100 |
| Ryanodine | Ryanodine receptor ligands | 100:1 to 1:120 |
| Spinertoram | Macrocyclic lactones | 150:1 to 1:100 |
| Spinosad | Macrocyclic lactones | 500:1 to 1:10 |
| Spirodiclofen | Lipid biosynthesis inhibitors | 200:1 to 1:200 |
| Spiromesifen | Lipid biosynthesis inhibitors | 200:1 to 1:200 |
| Tebufenozide | Ecdysone agonists | 500:1 to 1:250 |
| Thiacloprid | Neonicotinoids | 100:1 to 1:200 |
| Thiamethoxam | Neonicotinoids | 1250:1 to 1:1000 |
| Thiodicarb | Cholinesterase inhibitors | 500:1 to 1:400 |
| Thiosultap-sodium | Sodium channel modulators | 150:1 to 1:100 |
| Tralomethrin | Sodium channel modulators | 150:1 to 1:200 |
| Triazamate | Cholinesterase inhibitors | 250:1 to 1:100 |
| Triflumuron | Chitin synthesis inhibitors | 200:1 to 1:100 |
| Bacillus thuringiensis | Biological agents | 50:1 to 1:10 |
| Bacillus thuringiensis | Biological agents | 50:1 to 1:10 |
| delta-endotoxin | | |
| NPV (e.g., Gemstar) | Biological agents | 50:1 to 1:10 |

AVP Incorporation into Plants or Parts Thereof

The foregoing AVPs can be incorporated into plants, plant tissues, plant cells, and/or plant seeds, for either stable or transient expression of an AVP and/or the polynucleotide sequence that encodes for an AVP. In some embodiments, the AVP incorporated into a plant using recombinant techniques known in the art, may be in the form of an insecticidal protein which may comprise one or more AVP monomers, in addition to one or more non-AVP polypeptides or proteins, e.g. an endoplasmic reticulum signal peptide operably linked to one or more AVPs. As used herein with respect to transgenic plants, plant cells and plant seeds, the term “AVP” also encompasses an insecticidal protein comprising one or more AVPs in addition to one or more non-AVP peptides, polypeptides or proteins, and an “AVP polynucleotide” is similarly also used to encompass a polynucleotide or group of polynucleotides operable to express and/or encode an insecticidal protein comprising one or more AVPs in addition to one or more non-AVP polypeptides or proteins. The goal of incorporating an AVP into plants (i.e., to make transgenic plants that express Av3 variant polynucleotide and/or an AVP) is to deliver AVP containing insecticidal proteins to the pest via the insect's consumption of the transgenic AVP expressed in a plant tissue consumed by the insect. Upon this consumption of the AVP from its food, for example an insect feeding upon a transgenic plant, the consumed AVP may have the ability to inhibit the growth, impair the movement, or even kill an insect. Accordingly, transgenic plants expressing an AVP polynucleotide and/or polypeptide may express said AVP in a variety of tissues, including the epidermis (e.g., mesophyll); periderm; phloem; xylem; parenchyma; collenchyma; sclerenchyma; and primary and secondary meristematic tissues. For example, in some embodiments, a polynucleotide sequence encoding an AVP can be operably linked to a regulatory region containing a phosphoenolpyruvate carboxylase promoter, resulting in the expression of an AVP in a plant's mesophyll tissue.

Transgenic plants expressing an AVP and/or a polynucleotide operable to express AVP can be generated by any one of the various methods and protocols well known to those having ordinary skill in the art; such methods of the invention do not require that a particular method for introducing a nucleotide construct to a plant be used, only that the nucleotide construct gains access to the interior of at least one cell of the plant. Methods for introducing nucleotide constructs into plants are known in the art including, but not limited to, stable transformation methods, transient transformation methods, and virus-mediated methods. “Transgenic plants” or “transformed plants” or “stably transformed” plants or cells or tissues refers to plants that have incorporated or integrated exogenous nucleic acid sequences or DNA fragments into the plant cell. These nucleic acid sequences include those that are exogenous, or not present in the untransformed plant cell, as well as those that may be endogenous, or present in the untransformed plant cell. “Heterologous” generally refers to the nucleic acid sequences that are not endogenous to the cell or part of the native genome in which they are present, and have been added to the cell by infection, transfection, microinjection, electroporation, microprojection, or the like.

Transformation of plant cells can be accomplished by one of several techniques known in the art. Typically, a construct that expresses such a protein, for example, an AVP, would contain a promoter to drive transcription of the gene, as well as a 3′ untranslated region to allow transcription termination and polyadenylation. The design and organization of such constructs is well known in the art. In some embodiments, a gene can be engineered such that the resulting peptide is secreted, or otherwise targeted within the plant cell to a specific region and/or organelle. For example, the gene can be engineered to contain a signal peptide to facilitate transfer of the peptide to the endoplasmic reticulum. It may also be preferable to engineer the plant expression cassette to contain an intron, such that mRNA processing of the intron is required for expression.

Typically, a plant expression cassette can be inserted into a plant transformation vector. This plant transformation vector may be comprised of one or more DNA vectors needed for achieving plant transformation. For example, it is a common practice in the art to utilize plant transformation vectors that are comprised of more than one contiguous DNA segment. These vectors are often referred to in the art as “binary vectors.” Binary vectors as well as vectors with helper plasmids are most often used for Agrobacterium-mediated transformation, where the size and complexity of DNA segments needed to achieve efficient transformation is quite large, and it is advantageous to separate functions onto separate DNA molecules. Binary vectors typically contain a plasmid vector that contains the cis-acting sequences required for T-DNA transfer (such as left border and right border), a selectable marker that is engineered to be capable of expression in a plant cell, and a “gene of interest” (a gene engineered to be capable of expression in a plant cell for which generation of transgenic plants is desired). Also present on this plasmid vector are sequences required for bacterial replication. The cis-acting sequences are arranged in a fashion to allow efficient transfer into plant cells and expression therein. For example, the selectable marker gene and the AVP are located between the left and right borders. Often a second plasmid vector contains the trans-acting factors that mediate T-DNA transfer from Agrobacterium to plant cells. This plasmid often contains the virulence functions (Vir genes) that allow infection of plant cells by Agrobacterium, and transfer of DNA by cleavage at border sequences and vir-mediated DNA transfer, as is understood in the art (Hellens and Mullineaux (2000) Trends in Plant Science 5:446-451). Several types of Agrobacterium strains (e.g. LBA4404, GV3101, EHA101, EHA105, etc.) can be used for plant transformation. The second plasmid vector is not necessary for transforming the plants by other methods such as microprojection, microinjection, electroporation, polyethylene glycol, etc.

In general, plant transformation methods involve transferring heterologous DNA into target plant cells (e.g. immature or mature embryos, suspension cultures, undifferentiated callus, protoplasts, etc.), followed by applying a maximum threshold level of appropriate selection (depending on the selectable marker gene) to recover the transformed plant cells from a group of untransformed cell mass. Explants are typically transferred to a fresh supply of the same medium and cultured routinely. Subsequently, the transformed cells are differentiated into shoots after placing on regeneration medium supplemented with a maximum threshold level of selecting agent. The shoots are then transferred to a selective rooting medium for recovering rooted shoot or plantlet. The transgenic plantlet then grows into a mature plant and produces fertile seeds (e.g. Hiei et al. (1994) The Plant Journal 6:271-282; Ishida et al. (1996) Nature Biotechnology 14:745-750). Explants are typically transferred to a fresh supply of the same medium and cultured routinely. A general description of the techniques and methods for generating transgenic plants are found in Ayres and Park (1994) Critical Reviews in Plant Science 13:219-239 and Bommineni and Jauhar (1997) Maydica 42:107-120. Since the transformed material contains many cells; both transformed and non-transformed cells are present in any piece of subjected target callus or tissue or group of cells. The ability to kill non-transformed cells and allow transformed cells to proliferate results in transformed plant cultures. Often, the ability to remove non-transformed cells is a limitation to rapid recovery of transformed plant cells and successful generation of transgenic plants.

Transformation protocols as well as protocols for introducing nucleotide sequences into plants may vary depending on the type of plant or plant cell, i.e., monocot or dicot, targeted for transformation. Generation of transgenic plants may be performed by one of several methods, including, but not limited to, microinjection, electroporation, direct gene transfer, introduction of heterologous DNA by Agrobacterium into plant cells (Agrobacterium-mediated transformation), bombardment of plant cells with heterologous foreign DNA adhered to particles, ballistic particle acceleration, aerosol beam transformation (U.S. Published Application No. 20010026941; U.S. Pat. No. 4,945,050; International Publication No. WO 91/00915; U.S. Published Application No. 2002015066), Lee1 transformation, and various other non-particle direct-mediated methods to transfer DNA.

Methods for transformation of chloroplasts are known in the art. See, for example, Svab et al. (1990) Proc. Natl. Acad. Sci. USA 87:8526-8530; Svab and Maliga (1993) Proc. Natl. Acad. Sci. USA 90:913-917; Svab and Maliga (1993) EMBO J. 12:601-606. The method relies on particle gun delivery of DNA containing a selectable marker and targeting of the DNA to the plastid genome through homologous recombination. Additionally, plastid transformation can be accomplished by transactivation of a silent plastid-borne transgene by tissue-preferred expression of a nuclear-encoded and plastid-directed RNA polymerase. Such a system has been reported in McBride et al. (1994) Proc. Natl. Acad. Sci. USA 91:7301-7305.

Following integration of heterologous foreign DNA into plant cells, one then applies a maximum threshold level of appropriate selection in the medium to kill the untransformed cells and separate and proliferate the putatively transformed cells that survive from this selection treatment by transferring regularly to a fresh medium. By continuous passage and challenge with appropriate selection, one identifies and proliferates the cells that are transformed with the plasmid vector. Molecular and biochemical methods can then be used to confirm the presence of the integrated heterologous gene of interest into the genome of the transgenic plant.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present disclosure provides transformed seed (also referred to as “transgenic seed”) having a nucleotide construct of the invention, for example, an expression cassette of the invention, stably incorporated into their genome.

In various embodiments, the present disclosure provides an insecticidal protein comprising at least one AVP, that act as substrates for insect proteinases, proteases and peptidases (collectively referred to herein as “proteases”) as described above.

In some embodiments, transgenic plants or parts thereof, that may be receptive to the expression of AVPs and/or compositions comprising an AVP as described herein, can include: alfalfa, banana, barley, bean, broccoli, cabbage, canola, carrot, cassava, castor, cauliflower, celery, chickpea, Chinese cabbage, citrus, coconut, coffee, corn, clover, cotton, a cucurbit, cucumber, Douglas fir, eggplant, eucalyptus, flax, garlic, grape, hops, leek, lettuce, Loblolly pine, millets, melons, nut, oat, olive, onion, ornamental, palm, pasture grass, pea, peanut, pepper, pigeonpea, pine, potato, poplar, pumpkin, Radiata pine, radish, rapeseed, rice, rootstocks, rye, safflower, shrub, sorghum, Southern pine, soybean, spinach, squash, strawberry, sugar beet, sugarcane, sunflower, sweet corn, sweet gum, sweet potato, switchgrass, tea, tobacco, tomato, triticale, turf grass, watermelon, and a wheat plant. In some embodiments the transgenic plant may be grown from cells that were initially transformed with the DNA constructs described herein. In other embodiments, the transgenic plant may express the encoded AVP compositions in a specific tissue, or plant part, for example, a leaf, a stem a flower, a sepal, a fruit, a root, or a seed or combinations thereof.

In some embodiments, the plant, plant tissue, plant cell, or plant seed can be transformed with an AVP or a polynucleotide encoding the same, wherein the AVP comprises an AVP polypeptide with an the amino acid sequence X₁-X₂-C-C-P-C-Y-W-G-G-C-P-W-G-X₃-X₄-C-Y-P-X₅-G-C-X₆-X₇-X₈-X₉-X₁₀ (SEQ ID NO: 35); wherein X₁ is H, K, D, E, S, T, N, Q, C, G, P, A, V, I, L, M, F, Y, or W; X₂ is R, H, K, D, E, S, T, N, Q, C, G, P, A, V, I, L, M, F, Y, or W; X₃ is R, H, K, D, E, S, T, N, Q, C, G, P, A, V, I, L, M, F, Y, or W; X₄ is R, H, K, D, E, S, T, N, Q, C, G, P, A, V, I, L, M, F, Y, or W; X₅ is R, H, K, D, E, S, T, N, Q, C, G, P, A, V, I, L, M, F, Y, or W; X₆ is R, H, K, D, E, S, T, N, Q, C, G, P, A, V, I, L, M, F, Y, or W; X₇ is R, H, K, D, E, S, T, N, Q, C, G, P, A, V, I, L, M, F, Y, or W; X₈ is R, H, K, D, E, S, T, N, Q, C, G, P, A, V, I, L, M, F, Y, or W; X₉ is R, H, K, D, E, S, T, N, Q, C, G, P, A, V, I, L, M, F, Y, or W; and X₁₀ is R, H, K, D, E, S, T, N, Q, C, G, P, A, V, I, L, M, F, Y, W or absent.

In some embodiments, the plant, plant tissue, plant cell, or plant seed can be transformed with an AVP or a polynucleotide encoding the same, wherein the AVP comprises an AVP polypeptide with an the amino acid sequence X₁-X₂-C-C-P-C-Y-W-G-G-C-P-W-G-X₃-X₄-C-Y-P-X₅-G-C-X₆-X₇-X₈-X₉-X₁₀ (SEQ ID NO: 36); wherein X₁ is R, H, K, D, E, S, T, N, Q, C, G, P, A, V, I, L, M, F, Y, or W; X₂ is R, H, K, D, E, S, T, N, Q, C, G, P, A, V, I, L, M, F, Y, or W; X₃ is R, H, K, D, E, S, T, N, Q, C, G, P, A, V, I, L, M, F, Y, or W; X₄ is R, H, K, D, E, S, T, N, Q, C, G, P, A, V, I, L, M, F, Y, or W; X₅ is R, H, K, D, E, S, T, N, Q, C, G, P, A, V, I, L, M, F, Y, or W; X₆ is R, H, K, D, E, S, T, N, Q, C, G, P, A, V, I, L, M, F, Y, or W; X₇ is R, H, K, D, E, S, T, N, Q, C, G, P, A, V, I, L, M, F, Y, or W; X₈ is R, H, K, D, E, S, T, N, Q, C, G, P, A, V, I, L, M, F, Y, or W; X₉ is R, H, K, D, E, S, T, N, Q, C, G, P, A, V, I, L, M, F, Y, or W; and X₁₀ is absent.

In some embodiments, the plant, plant tissue, plant cell, or plant seed can be transformed with an AVP or a polynucleotide encoding the same, wherein the AVP comprises an AVP polypeptide with an the amino acid sequence “KACCPCYWGGCPWGAACYPAGCAAAK” (e.g., SEQ ID NO:30).

In some embodiments, a plant, plant tissue, plant cell, or plant seed can be transformed with an AVP or a polynucleotide encoding AVP, wherein the AVP comprises an AVP polypeptide with an amino acid sequence of X₁-X₂-C-C-P-C-Y-W-G-G-C-P-W-G-X₃—X₄-C-Y-P-X₅-G-C-X₆-X₇-X₈-X₉-X₁₀ (SEQ ID NO: 35); wherein X₁ is H, K, D, E, S, T, N, Q, C, G, P, A, V, I, L, M, F, Y, or W; X₂ is R, H, K, D, E, S, T, N, Q, C, G, P, A, V, I, L, M, F, Y, or W; X₃ is R, H, K, D, E, S, T, N, Q, C, G, P, A, V, I, L, M, F, Y, or W; X₄ is R, H, K, D, E, S, T, N, Q, C, G, P, A, V, I, L, M, F, Y, or W; X₅ is R, H, K, D, E, S, T, N, Q, C, G, P, A, V, I, L, M, F, Y, or W; X₆ is R, H, K, D, E, S, T, N, Q, C, G, P, A, V, I, L, M, F, Y, or W; X₇ is R, H, K, D, E, S, T, N, Q, C, G, P, A, V, I, L, M, F, Y, or W; X₈ is R, H, K, D, E, S, T, N, Q, C, G, P, A, V, I, L, M, F, Y, or W; X₉ is R, H, K, D, E, S, T, N, Q, C, G, P, A, V, I, L, M, F, Y, or W; and X₁₀ is R, H, K, D, E, S, T, N, Q, C, G, P, A, V, I, L, M, F, Y, W or absent.

In some embodiments, a plant, plant tissue, plant cell, or plant seed can be transformed with an AVP or a polynucleotide encoding AVP, wherein the AVP comprises an AVP polypeptide with an amino acid sequence of X₁-X₂-C-C-P-C-Y-W-G-G-C-P-W-G-X₃—X₄-C-Y-P-X₅-G-C-X₆-X₇-X₈-X₉-X₁₀ (SEQ ID NO: 36); wherein X₁ is R, H, K, D, E, S, T, N, Q, C, G, P, A, V, I, L, M, F, Y, or W; X₂ is R, H, K, D, E, S, T, N, Q, C, G, P, A, V, I, L, M, F, Y, or W; X₃ is R, H, K, D, E, S, T, N, Q, C, G, P, A, V, I, L, M, F, Y, or W; X₄ is R, H, K, D, E, S, T, N, Q, C, G, P, A, V, I, L, M, F, Y, or W; X₅ is R, H, K, D, E, S, T, N, Q, C, G, P, A, V, I, L, M, F, Y, or W; X₆ is R, H, K, D, E, S, T, N, Q, C, G, P, A, V, I, L, M, F, Y, or W; X₇ is R, H, K, D, E, S, T, N, Q, C, G, P, A, V, I, L, M, F, Y, or W; X₈ is R, H, K, D, E, S, T, N, Q, C, G, P, A, V, I, L, M, F, Y, or W; X₉ is R, H, K, D, E, S, T, N, Q, C, G, P, A, V, I, L, M, F, Y, or W; and X₁₀ is absent.

In some embodiments, a plant, plant tissue, plant cell, or plant seed can be transformed with an AVP or a polynucleotide encoding AVP, wherein the AVP comprises an AVP polypeptide with an amino acid sequence of X₁-X₂-C-C-P-C-Y-W-G-G-C-P-W-G-X₃—X₄-C-Y-P-X₅-G-C-X₆-X₇-X₈-X₉-X₁₀ (SEQ ID NO: 35); wherein X₁ is H, K, D, E, S, T, N, Q, C, G, P, A, V, I, L, M, F, Y, or W; X₂ is R, H, K, D, E, S, T, N, Q, C, G, P, A, V, I, L, M, F, Y, or W; X₃ is R, H, K, D, E, S, T, N, Q, C, G, P, A, V, I, L, M, F, Y, or W; X₄ is R, H, K, D, E, S, T, N, Q, C, G, P, A, V, I, L, M, F, Y, or W; X₅ is R, H, K, D, E, S, T, N, Q, C, G, P, A, V, I, L, M, F, Y, or W; X₆ is R, H, K, D, E, S, T, N, Q, C, G, P, A, V, I, L, M, F, Y, or W; X₇ is R, H, K, D, E, S, T, N, Q, C, G, P, A, V, I, L, M, F, Y, or W; X₈ is R, H, K, D, E, S, T, N, Q, C, G, P, A, V, I, L, M, F, Y, or W; X₉ is R, H, K, D, E, S, T, N, Q, C, G, P, A, V, I, L, M, F, Y, or W; and X₁₀ is R, H, K, D, E, S, T, N, Q, C, G, P, A, V, I, L, M, F, Y, W or absent; X₁-X₂-C-C-P-C-Y-W-G-G-C-P-W-G-X₃-X₄-C-Y-P-X₅-G-C-X₆-X₇-X₈-X₉-X₁₀ (SEQ ID NO: 36); wherein X₁ is R, H, K, D, E, S, T, N, Q, C, G, P, A, V, I, L, M, F, Y, or W; X₂ is R, H, K, D, E, S, T, N, Q, C, G, P, A, V, I, L, M, F, Y, or W; X₃ is R, H, K, D, E, S, T, N, Q, C, G, P, A, V, I, L, M, F, Y, or W; X₄ is R, H, K, D, E, S, T, N, Q, C, G, P, A, V, I, L, M, F, Y, or W; X₅ is R, H, K, D, E, S, T, N, Q, C, G, P, A, V, I, L, M, F, Y, or W; X₆ is R, H, K, D, E, S, T, N, Q, C, G, P, A, V, I, L, M, F, Y, or W; X₇ is R, H, K, D, E, S, T, N, Q, C, G, P, A, V, I, L, M, F, Y, or W; X₈ is R, H, K, D, E, S, T, N, Q, C, G, P, A, V, I, L, M, F, Y, or W; X₉ is R, H, K, D, E, S, T, N, Q, C, G, P, A, V, I, L, M, F, Y, or W; and X₁₀ is absent; and/or any combinations thereof.

(e.g., SEQ ID NO: 30)
“KACCPCYWGGCPWGAACYPAGCAAAK”.

In some embodiments, a plant, plant tissue, plant cell, or plant seed can be transformed with an one or more AVPs, e.g., in some embodiments, the plant, plant tissue, plant cell, or plant seed can be transformed with one or more AVPs with the amino acid sequence

In some embodiments, the plant, plant tissue, plant cell or seed may be transformed with a combinations of AVPs, e.g., a group consisting of an AVP with the amino acid sequence of X₁-X₂-C-C-P-C-Y-W-G-G-C-P-W-G-X₃-X₄-C-Y-P-X₅-G-C-X₆-X₇-X₈-X₉-X₁₀ (SEQ ID NO: 35); wherein X₁ is H, K, D, E, S, T, N, Q, C, G, P, A, V, I, L, M, F, Y, or W; X₂ is R, H, K, D, E, S, T, N, Q, C, G, P, A, V, I, L, M, F, Y, or W; X₃ is R, H, K, D, E, S, T, N, Q, C, G, P, A, V, I, L, M, F, Y, or W; X₄ is R, H, K, D, E, S, T, N, Q, C, G, P, A, V, I, L, M, F, Y, or W; X₅ is R, H, K, D, E, S, T, N, Q, C, G, P, A, V, I, L, M, F, Y, or W; X₆ is R, H, K, D, E, S, T, N, Q, C, G, P, A, V, I, L, M, F, Y, or W; X₇ is R, H, K, D, E, S, T, N, Q, C, G, P, A, V, I, L, M, F, Y, or W; X₈ is R, H, K, D, E, S, T, N, Q, C, G, P, A, V, I, L, M, F, Y, or W; X₉ is R, H, K, D, E, S, T, N, Q, C, G, P, A, V, I, L, M, F, Y, or W; and X₁₀ is R, H, K, D, E, S, T, N, Q, C, G, P, A, V, I, L, M, F, Y, W or absent; X₁-X₂-C-C-P-C-Y-W-G-G-C-P-W-G-X₃-X₄-C-Y-P-X₅-G-C-X₆-X₇-X₈-X₉-X₁₀ (SEQ ID NO: 36); wherein X₁ is R, H, K, D, E, S, T, N, Q, C, G, P, A, V, I, L, M, F, Y, or W; X₂ is R, H, K, D, E, S, T, N, Q, C, G, P, A, V, I, L, M, F, Y, or W; X₃ is R, H, K, D, E, S, T, N, Q, C, G, P, A, V, I, L, M, F, Y, or W; X₄ is R, H, K, D, E, S, T, N, Q, C, G, P, A, V, I, L, M, F, Y, or W; X₅ is R, H, K, D, E, S, T, N, Q, C, G, P, A, V, I, L, M, F, Y, or W; X₆ is R, H, K, D, E, S, T, N, Q, C, G, P, A, V, I, L, M, F, Y, or W; X₇ is R, H, K, D, E, S, T, N, Q, C, G, P, A, V, I, L, M, F, Y, or W; X₈ is R, H, K, D, E, S, T, N, Q, C, G, P, A, V, I, L, M, F, Y, or W; X₉ is R, H, K, D, E, S, T, N, Q, C, G, P, A, V, I, L, M, F, Y, or W; and X₁₀ is absent; and/or any combinations thereof.

In some embodiments, the plant, plant tissue, plant cell or seed may be transformed with a polynucleotide operable to encode an AVP selected from any one of the following amino acid sequences: X₁-X₂-C-C-P-C-Y-W-G-G-C-P-W-G-X₃-X₄-C-Y-P-X₅-G-C-X₆-X₇-X₈-X₉-X₁₀ (SEQ ID NO: 35); wherein X₁ is H, K, D, E, S, T, N, Q, C, G, P, A, V, I, L, M, F, Y, or W; X₂ is R, H, K, D, E, S, T, N, Q, C, G, P, A, V, I, L, M, F, Y, or W; X₃ is R, H, K, D, E, S, T, N, Q, C, G, P, A, V, I, L, M, F, Y, or W; X₄ is R, H, K, D, E, S, T, N, Q, C, G, P, A, V, I, L, M, F, Y, or W; X₅ is R, H, K, D, E, S, T, N, Q, C, G, P, A, V, I, L, M, F, Y, or W; X₆ is R, H, K, D, E, S, T, N, Q, C, G, P, A, V, I, L, M, F, Y, or W; X₇ is R, H, K, D, E, S, T, N, Q, C, G, P, A, V, I, L, M, F, Y, or W; X₈ is R, H, K, D, E, S, T, N, Q, C, G, P, A, V, I, L, M, F, Y, or W; X₉ is R, H, K, D, E, S, T, N, Q, C, G, P, A, V, I, L, M, F, Y, or W; and X₁₀ is R, H, K, D, E, S, T, N, Q, C, G, P, A, V, I, L, M, F, Y, W or absent; X₁-X₂-C-C-P-C-Y-W-G-G-C-P-W-G-X₃-X₄-C-Y-P-X₅-G-C-X₆-X₇-X₈-X₉-X₁₀ (SEQ ID NO: 36); wherein X₁ is R, H, K, D, E, S, T, N, Q, C, G, P, A, V, I, L, M, F, Y, or W; X₂ is R, H, K, D, E, S, T, N, Q, C, G, P, A, V, I, L, M, F, Y, or W; X₃ is R, H, K, D, E, S, T, N, Q, C, G, P, A, V, I, L, M, F, Y, or W; X₄ is R, H, K, D, E, S, T, N, Q, C, G, P, A, V, I, L, M, F, Y, or W; X₅ is R, H, K, D, E, S, T, N, Q, C, G, P, A, V, I, L, M, F, Y, or W; X₆ is R, H, K, D, E, S, T, N, Q, C, G, P, A, V, I, L, M, F, Y, or W; X₇ is R, H, K, D, E, S, T, N, Q, C, G, P, A, V, I, L, M, F, Y, or W; X₈ is R, H, K, D, E, S, T, N, Q, C, G, P, A, V, I, L, M, F, Y, or W; X₉ is R, H, K, D, E, S, T, N, Q, C, G, P, A, V, I, L, M, F, Y, or W; and X₁₀ is absent; and/or any combinations thereof.

In some embodiments, the insecticidal protein can have a cleavable peptide fused in frame with the AVP. In another embodiment, the insecticidal protein can have two or more cleavable peptides, wherein the insecticidal protein comprises an insect cleavable linker (L), the insect cleavable linker being fused in frame with a construct comprising (AVP-L)ₙ, wherein n is an integer ranging from 1 to 200, or from 1 to 100, or from 1 to 10. In another embodiment, the insecticidal protein described herein comprises an endoplasmic reticulum signal peptide (ERSP) fused in frame with an AVP, which is fused in frame with an insect cleavable linker (L) and/or a repeat construct (L-AVP)ₙ or (AVP-L)ₙ, wherein n is an integer ranging from 1 to 200, or from 1 to 100, or from 1 to 10. In various embodiments, an exemplary insecticidal protein can include a protein construct comprising: (ERSP)-(AVP-L)ₙ, or (ERSP)-(L)-(AVP-L)ₙ, or (ERSP)-(L-AVP)ₙ, or (ERSP)-(L-AVP)ₙ-(L), wherein n is an integer ranging from 1 to 200 or from 1 to 100, or from 1 to 10. In various related embodiments described above, an AVP is the aforementioned Av3 variant polypeptide, L is an insect cleavable peptide, and n is an integer ranging from 1 to 200, preferably an integer ranging from 1 to 100, and more preferably an integer ranging from 1 to 10. In some embodiments, the insecticidal protein may contain AVP peptides that are the same or different, and insect cleavable peptides that are the same or different. In some embodiments, the C-terminal AVP is fused or unfused at its C-terminus with an insect cleavable peptide. In some embodiments, the N-terminal AVP is fused or unfused at its N-terminus with an insect cleavable peptide.

Some of the available proteases and peptidases found in insect gut environment are dependent on the life-stage of the insect as these enzymes are often spatially and temporally expressed. The digestive system of the insect is composed of the alimentary canal and associated glands. Food enters the mouth and is mixed with secretions that may or may not contain digestive proteases and peptidases. The foregut and the hind gut are ectodermal in origin. The foregut serves generally as a storage depot for raw food. From the foregut discrete packages of food pass into the midgut (mesenteron or ventriculus). The midgut is the site of digestion and absorption of food nutrients. Generally, the presence of certain proteases and peptidases in the midgut follow the pH of the gut. Certain proteases and peptidases in the human gastrointestinal system may include: pepsin, trypsin, chymotrypsin, elastase, carboxypeptidase, aminopeptidase, and dipeptidase. Insect gut environment, include the regions of the digestive system in the herbivore species where peptides and proteins are degraded during digestion. Some of the available proteases and peptidases found in insect gut environments may include: (1) serine proteases; (2) cysteine proteases; (3) aspartic proteases, and (4) metalloproteases.

The two predominant protease classes in the digestive systems of phytophagous insects are the serine and cysteine proteases. Murdock et al. (1987) carried out an elaborate study of the midgut enzymes of various pests belonging to Coleoptera, while Srinivasan et al. (2008) have reported on the midgut enzymes of various pests belonging to Lepidoptera. Serine proteases are known to dominate the larval gut environment and contribute to about 95% of the total digestive activity in Lepidoptera, whereas the Coleopteran species have a wider range of dominant gut proteases, including cysteine proteases. The papain family contains peptidases with a wide variety of activities, including endopeptidases with broad specificity (such as papain), endopeptidases with very narrow specificity (such as glycyl endopeptidases), aminopeptidases, dipeptidyl-peptidase, and peptidases with both endopeptidase and exopeptidase activities (such as cathepsins B and H). Other exemplary proteinases found in the midgut of various insects include trypsin-like enzymes, e.g. trypsin and chymotrypsin, pepsin, carboxypeptidase-B and aminotripeptidases.

Serine proteases are widely distributed in nearly all animals and microorganisms (Joanitti et al., 2006). In higher organisms, nearly 2% of genes code for these enzymes (Barrette-Ng et al., 2003). Being essentially indispensable to the maintenance and survival of their host organism, serine proteases play key roles in many biological processes. Serine proteases are classically categorized by their substrate specificity, notably by whether the residue at P1: trypsin-like (Lys/Arg preferred at P1), chymotrypsin-like (large hydrophobic residues such as Phe/Tyr/Leu at P1), or elastase-like (small hydrophobic residues such as Ala/Val at P1) (revised by Tyndall et. al., 2005). Serine proteases are a class of proteolytic enzymes whose central catalytic machinery is composed of three invariant residues, an aspartic acid, a histidine and a uniquely reactive serine, the latter giving rise to their name, the “catalytic triad”. The Asp-His-Ser triad can be found in at least four different structural contexts (Hedstrom, 2002). These four clans of serine proteases are typified by chymotrypsin, subtilisin, carboxypeptidase Y, and Clp protease. The three serine proteases of the chymotrypsin-like clan that have been studied in greatest detail are chymotrypsin, trypsin, and elastase. More recently, serine proteases with novel catalytic triads and dyads have been discovered for their roles in digestion, including Ser-His-Glu, Ser-Lys/His, His-Ser-His, and N-terminal Ser.

One class of well-studied digestive enzymes found in the gut environment of insects is the class of cysteine proteases. The term “cysteine protease” is intended to describe a protease that possesses a highly reactive thiol group of a cysteine residue at the catalytic site of the enzyme. There is evidence that many phytophagous insects and plant parasitic nematodes rely, at least in part, on midgut cysteine proteases for protein digestion. These include but are not limited to Hemiptera, especially squash bugs (Anasa tristis); green stink bug (Acrosternum hilare); Riptortus clavatus; and almost all Coleoptera examined to date, especially, Colorado potato beetle (Leptinotarsa deaemlineata); three-lined potato beetle (Lema trilineata); asparagus beetle (Crioceris asparagi); Mexican bean beetle (Epilachna varivestis); red flour beetle (Triolium castaneum); confused flour beetle (Tribolium confusum); the flea beetles (Chaetocnema spp., Haltica spp., and Epitrix spp.); corn rootworm (Diabrotica Spp.); cowpea weevil (Callosobruchus aculatue); boll weevil (Antonomus grandis); rice weevil (Sitophilus oryza); maize weevil (Sitophilus zeamais); granary weevil (Sitophilus granarius); Egyptian alfalfa weevil (Hypera postica); bean weevil (Acanthoseelides obtectus); lesser grain borer (Rhyzopertha dominica); yellow meal worm (Tenebrio molitor); Thysanoptera, especially, western flower thrips (Franklini ella occidentalis); Diptera, especially, leafminer spp. (Liriomyza trifolii); plant parasitic nematodes especially the potato cyst nematodes (Globodera spp.), the beet cyst nematode (Heterodera schachtii) and root knot nematodes (Meloidogyne spp.).

Another class of digestive enzymes is the aspartic proteases. The term “aspartic protease” is intended to describe a protease that possesses two highly reactive aspartic acid residues at the catalytic site of the enzyme and which is most often characterized by its specific inhibition with pepstatin, a low molecular weight inhibitor of nearly all known aspartic proteases. There is evidence that many phytophagous insects rely, in part, on midgut aspartic proteases for protein digestion most often in conjunction with cysteine proteases. These include but are not limited to Hemiptera especially (Rhodnius prolixus) and bedbug (Cimex spp.) and members of the families Phymatidae, Pentatomidae, Lygaeidae and Belostomatidae; Coleoptera, in the families of the Meloidae, Chrysomelidae, Coccinelidae and Bruchidae all belonging to the series Cucujiformia, especially, Colorado potato beetle (Leptinotarsa decemlineata) three-lined potato beetle (Lematri lineata); southern and western corn rootworm (Diabrotica undecimpunctata and D. virgifera), boll weevil (Anthonomus grandis), squash bug (Anasatristis); flea beetle (Phyllotreta crucifera), bruchid beetle (Callosobruchus maculatus), Mexican bean beetle (Epilachna varivestis), soybean leafminer (Odontota horni), margined blister beetle (Epicauta pestifera) and the red flour beetle (Triolium castaneum); Diptera, especially housefly (Musca domestica) (Terra and Ferreira (1994) Comn. Biochem. Physiol. 109B: 1-62; Wolfson and Murdock (1990) J. Chem. Ecol. 16: 1089-1102).

In some embodiments, the present disclosure comprises a method for controlling an invertebrate pest in agronomic and/or nonagronomic applications, comprising contacting the invertebrate pest or its environment, a solid surface, including a plant surface or part thereof, with a biologically effective amount of one or more of the AVPs of the invention, or with an insecticidal protein comprising at least one AVP or a composition comprising at least one or more of the AVPs of the invention, or an insecticidal protein comprising at least one AVP, and a biologically effective amount of at least one SIPCA. Examples of suitable compositions comprising at least one or more of the AVPs of the invention, or an insecticidal protein comprising at least one AVP, or at least one or more of the AVPs of the invention, or an insecticidal protein comprising at least one AVP, and a biologically effective amount of at least one SIPCA, include a liquid solution, an emulsion, a powder, a granule, a nanoparticle, a microparticle, or a combination of the above formulated into a compositions wherein the additional SIPCA is present on or in the same composition as the AVP or insecticidal protein comprising an AVP of the invention, for example, a part of a granular composition or on granules separate from those of the AVP polypeptide or insecticidal of the invention.

In some embodiments, to achieve contact with a compound or composition of the invention to protect a field crop from invertebrate pests, the compound or composition is typically applied to the seed of the crop before planting, to the foliage (e.g., leaves, stems, flowers, fruits) of crop plants, or to the soil or other growth medium before or after the crop is planted.

One embodiment of a method of contact is by spraying. Alternatively, a granular composition comprising an AVP or insecticidal protein comprising one or more AVPs of the invention can be applied to the plant foliage or the soil. Compounds of this invention can also be effectively delivered through plant uptake by contacting the plant with a composition comprising a compound of this invention applied as a soil drench of a liquid formulation, a granular formulation to the soil, a nursery box treatment or a dip of transplants. Of note is a composition of the present disclosure in the form of a soil drench liquid formulation. Also of note is a method for controlling an invertebrate pest comprising contacting the invertebrate pest or its environment with a biologically effective amount of an AVP or insecticidal protein comprising one or more AVPs of the invention of the present disclosure, or with a composition comprising a biologically effective amount of an AVP or insecticidal protein comprising one or more AVPs of the invention of the present disclosure. Of further note, in some illustrative embodiments, the illustrative method includes wherein the environment is soil and the composition is applied to the soil as a soil drench formulation. Of further note is that an AVP or insecticidal protein comprising one or more AVPs of the invention are also effective by localized application to the locus of infestation. Other methods of contact include application of a compound or a composition of the invention by direct and residual sprays, aerial sprays, gels, seed coatings, microencapsulations, systemic uptake, baits, ear tags, boluses, foggers, fumigants, aerosols, dusts and many others. One embodiment of a method of contact is a dimensionally stable fertilizer granule, stick or tablet comprising a compound or composition of the invention. The compounds of this invention can also be impregnated into materials for fabricating invertebrate control devices (e.g., insect netting, application onto clothing, application into candle formulations and the like).

In some embodiments, an AVP or insecticidal protein comprising one or more AVPs of the invention are also useful in seed treatments for protecting seeds from invertebrate pests. In the context of the present disclosure and claims, treating a seed means contacting the seed with a biologically effective amount of an AVP or insecticidal protein comprising one or more AVPs of the invention of this invention, which is typically formulated as a composition of the invention. This seed treatment protects the seed from invertebrate soil pests and generally can also protect roots and other plant parts in contact with the soil of the seedling developing from the germinating seed. The seed treatment may also provide protection of foliage by translocation of the AVP or insecticidal protein comprising one or more AVPs of the invention and/or a SIPCA within the developing plant. Seed treatments can be applied to all types of seeds, including those from which plants genetically transformed to express specialized traits will germinate. In addition, an AVP or insecticidal protein comprising one or more AVPs of the invention can be transformed into a plant or part thereof, for example a plant cell, or plant seed, that is already transformed with proteins toxic to invertebrate pests, such as Bacillus thuringiensis toxins or protein crystals or those expressing herbicide resistance such as glyphosate acetyltransferase, which provides resistance to glyphosate. Representative examples include those expressing proteins toxic to invertebrate pests, such as Bacillus thuringiensis toxins or protein crystals or those expressing herbicide resistance such as glyphosate acetyltransferase, which provides resistance to glyphosate.

One method of seed treatment is by spraying or dusting the seed with an AVP or insecticidal protein comprising one or more AVPs of the invention (i.e. as a formulated composition) before sowing the seeds. Compositions formulated for seed treatment generally comprise a film former or adhesive agent. Therefore typically a seed coating composition of the present disclosure comprises a biologically effective amount of an AVP or insecticidal protein comprising one or more AVPs of the invention, and a film former or adhesive agent. Seed can be coated by spraying a flowable suspension concentrate directly into a tumbling bed of seeds and then drying the seeds. Alternatively, other formulation types such as wetted powders, solutions, suspoemulsions, emulsifiable concentrates and emulsions in water can be sprayed on the seed. This process is particularly useful for applying film coatings on seeds. Various coating machines and processes are available to one skilled in the art. Suitable processes include those listed in P. Kosters et al., Seed Treatment: Progress and Prospects, 1994 BCPC Monograph No. 57, and references listed therein.

The treated seed typically comprises an AVP or insecticidal protein comprising one or more AVPs of the invention in an amount ranging from about 0.01 g to 1 kg per 100 kg of seed (i.e. from about 0.00001 to 1% by weight of the seed before treatment). A flowable suspension formulated for seed treatment typically comprises from about 0.5 to about 70% of the active ingredient, from about 0.5 to about 30% of a film-forming adhesive, from about 0.5 to about 20% of a dispersing agent, from 0 to about 5% of a thickener, from 0 to about 5% of a pigment and/or dye, from 0 to about 2% of an antifoaming agent, from 0 to about 1% of a preservative, and from 0 to about 75% of a volatile liquid diluent.

A challenge regarding the expression of heterogeneous polypeptides in transgenic plants is maintaining the desired effect (e.g., insecticidal activity) of the introduced polypeptide upon expression in the host organism; one way to maintain such an effect is to increase the chance of proper protein folding through the use of an operably linked Endoplasmic Reticulum Signal Peptide (ERSP). Another method to maintain the effect of a transgenic protein is to incorporate a Translational Stabilizing Protein (STA). In some embodiments, a peptide comprised of an Endoplasmic Reticulum Signal Peptide (ERSP) can be operably linked to an AVP (designated as ERSP-AVP), wherein said ERSP is the N-terminal of said peptide, and where the ERSP peptide is between 3 to 60 amino acids in length, between 5 to 50 amino acids in length, between 20 to 30 amino acids in length and or where the peptide is BAAS, or tobacco extensin signal peptide, or a modified tobacco extensin signal peptide, or Jun a 3 signal peptide of Juniperus ashei. For example, in some embodiments, a plant can be transformed with a nucleotide that codes for any of the peptides that are described herein as Endoplasmic Reticulum Signal Peptides (ERSP) and/or an AVP.

In some embodiments, a protein comprised of an Endoplasmic Reticulum Signal Peptide (ERSP) can be operably linked to an AVP, operably linked to an intervening linker peptide (L or Linker), designated as ERSP-Linker-AVP, (ERSP-L-AVP), or ERSP-AVP-Linker (ERSP-AVP-L), wherein said ERSP is the N-terminal of said protein and said L or Linker, may be either on the N-terminal side (upstream) of the AVP or the C-terminal side (downstream) of AVP. A protein designated as ERSP-L-AVP, or ERSP-AVP-L, comprising any of the ERSPs or AVPs described herein and wherein said L can be an uncleavable linker peptide, or a cleavable linker peptide, which may be cleavable in a plant cells during protein expression process or may be cleavable in an insect gut environments and hemolymph environments, and comprised of any of the intervening linker peptide (LINKER) described, or taught by this document including the following sequences: IGER (SEQ ID NO:6), EEKKN, (SEQ ID NO:7), and ETMFKHGL (SEQ ID NO:8).

In some embodiments, a protein comprising an Endoplasmic Reticulum Signal Peptide (ERSP) operably linked to an AVP operably linked to a Translational Stabilizing Protein (STA), designated as ERSP-STA-AVP or ERSP-AVP-STA, wherein said ERSP is the N-terminal of said protein and said STA may be either on the N-terminal side (upstream) of the AVP, or of the C-terminal side (downstream) of AVP. In some embodiments, a protein designated as ERSP-STA-AVP or ERSP-AVP-STA, comprising any of the ERSPs or AVPs described herein, can be operably linked to a STA, for example, any of the translational stabilizing proteins described, or taught by this document including GFP (Green Fluorescent Protein; SEQ ID NO:9; NCBI Accession No. AAF65230.1), GNA (snowdrop lectin SEQ ID NO:10; NCBI Accession No. AAL07474.1), Jun a 3, (Juniperus ashei; SEQ ID NO:11; NCBI Accession No. P81295.1).

Av3 Variant Polynucleotide Incorporation into Plants

Plants can be transiently or stably transfected with the DNA sequence that encodes an AVP, or an insecticidal protein comprising one or more AVPs and one or more non-AVP peptides, polypeptides or proteins, for example, using anyone of the transfection methods described above; alternatively, plants can be transfected with a polynucleotide that encodes AVP operably linked to an ERSP, LINKER, and/or a STA protein encoding polynucleotide. For example, in some embodiments, a transgenic plant or plant genome can be transfected to incorporate the polynucleotide sequence that encodes the Endoplasmic Reticulum Signal Peptide (ERSP), AVP and/or intervening linker peptide (LINKER, L), thus causing mRNA transcribed from the heterogeneous DNA to be expressed in the transformed plant.

The present disclosure may be used for transformation of any plant species, including, but not limited to, monocots and dicots. Crops for which a transgenic approach or PEP would be an especially useful approach include, but are not limited to: alfalfa, cotton, tomato, maize, wheat, corn, sweet corn, lucerne, soybean, sorghum, field pea, linseed, safflower, rapeseed, oil seed rape, rice, soybean, barley, sunflower, trees (including coniferous and deciduous), flowers (including those grown commercially and in greenhouses), field lupins, switchgrass, sugarcane, potatoes, tomatoes, tobacco, crucifers, peppers, sugarbeet, barley, and oilseed rape, Brassica sp., rye, millet, peanuts, sweet potato, cassava, coffee, coconut, pineapple, citrus trees, cocoa, tea, banana, avocado, fig, guava, mango, olive, papaya, cashew, macadamia, almond, oats, vegetables, ornamentals, and conifers.

In some embodiments, the AVP expression open reading frame (ORF) described herein is a polynucleotide sequence which will enable the plant to express mRNA, which in turn will be translated into peptides be expressed, folded properly, and/or accumulated to such an extent that said proteins provide a dose sufficient to inhibit and/or kill one or more pests. In one embodiment, an example of a protein AVP expression ORF can be an Av3 variant polynucleotide, an “ersp” (i.e., the polynucleotide sequence that encodes the ERSP polypeptide) a “linker” (i.e., the polynucleotide sequence that encodes the LINKER polypeptide), a “sta” (i.e., the polynucleotide sequence that encodes the STA polypeptide), or any combination thereof, and can be described in the following equation format:
ersp-sta-(linkerᵢ-AVPⱼ)_{N}, or ersp-(AVPⱼ-linkerᵢ)_{N}-sta

The foregoing illustrative embodiment of a polynucleotide equation would result in the following protein complex being expressed: ERSP-STA-(LINKER_{I}-AVP_{J})_{N}, containing four possible peptide components with dash signs to separate each component. The nucleotide component of ersp is a polynucleotide segment encoding a plant endoplasmic reticulum trafficking signal peptide (ERSP). The component of sta is a polynucleotide segment encoding a translation stabilizing protein (STA), which helps the accumulation of the AVP expressed in plants, however, in some embodiments, the inclusion of sta may not be necessary in the AVP expression ORF. The component of linker₁ is a polynucleotide segment encoding an intervening linker peptide (L OR LINKER) to separate the AVP from other components contained in ORF, and from the translation stabilizing protein. The subscript letter “i” indicates that in some embodiments, different types of linker peptides can be used in the AVP expression ORF. The component “avp” indicates the polynucleotide segment encoding the AVP (also known as the Av3 variant polynucleotide sequence). The subscript “j” indicates different Av3 variant polynucleotides may be included in the AVP expression ORF. For example, in some embodiments, the Av3 variant polynucleotide sequence can encode an AVP with an amino acid substitution, or an amino acid deletion. The subscript “N” as shown in “(linkerᵢ-avpⱼ)_{N}” indicates that the structure of the nucleotide encoding an intervening linker peptide and an AVP can be repeated “N” times in the same open reading frame in the same AVP expression ORF, where N can be any integrate number from 1 to 10. N can be from 1 to 10, specifically N can be 1, 2, 3, 4, or 5, and in some embodiments N is 6, 7, 8, 9 or 10. The repeats may contain polynucleotide segments encoding different intervening linkers (LINKER) and different AVPs. The different polynucleotide segments including the repeats within the same AVP expression ORF are all within the same translation frame. In some embodiments, the inclusion of a sta polynucleotide in the AVP expression ORF may not be required. For example, an, ersp polynucleotide sequence can be directly be linked to the polynucleotide encoding an Av3 variant polynucleotide without a linker.

In the foregoing exemplary equation, the polynucleotide “avp” encoding the polypeptide “AVP” can be the polynucleotide sequence that encodes any variant Av3 polypeptide. For example, in some embodiments, the “avp” polynucleotide can encode an AVP having an N-terminal mutation, for example, an N-terminal amino acid substitution of RIK relative to SEQ ID NO:1, (e.g. polypeptide Av3a) changing the polypeptide sequence from the wild-type “RSCCPCYWGGCPWGQNCYPEGCSGPKV” (SEQ ID NO: 1) to “KSCCPCYWGGCPWGQNCYPEGCSGPKV” (SEQ ID NO:2).

In some embodiments, the “avp” polynucleotide can encode an AVP having a C-terminal mutation (e.g., polypeptide Av3a-C1), for example, a C-terminal amino acid deletion relative to SEQ ID NO: 1, changing the polypeptide sequence from the wild-type “RSCCPCYWGGCPWGQNCYPEGCSGPKV” (SEQ ID NO: 1) to “RSCCPCYWGGCPWGQNCYPEGCSGPK” (SEQ ID NO:3).

In some embodiments, the “avp” polynucleotide can encode an N-terminal mutation and a C-terminal mutation, for example, an N-terminal amino acid substitution of RIK relative to SEQ ID NO:1, and a C-terminal amino acid deletion to SEQ ID NO:1, (i.e. polypeptide Av3b) changing the polypeptide sequence from the wild-type “RSCCPCYWGGCPWGQNCYPEGCSGPKV” (SEQ ID NO: 1) to “KSCCPCYWGGCPWGQNCYPEGCSGPK” (SEQ ID NO:4).

In some embodiments, AVP expression ORF starts with an ersp at its 5′-end. For the AVP to be properly folded and functional when it is expressed from a transgenic plant, it must have an ersp nucleotide fused in frame with the polynucleotide encoding an AVP. During the cellular translation process, translated ERSP can direct the AVP being translated to insert into the Endoplasmic Reticulum (ER) of the plant cell by binding with a cellular component called a signal-recognition particle. Within the ER the ERSP peptide is cleaved by signal peptidase and the AVP is released into the ER, where the AVP is properly folded during the post-translation modification process, for example, the formation of disulfide bonds. Without any additional retention protein signals, the protein is transported through the ER to the Golgi apparatus, where it is finally secreted outside the plasma membrane and into the apoplastic space. AVP can accumulate at apoplastic space efficiently to reach the insecticidal dose in plants.

The ERSP peptide is at the N-terminal region of the plant translated AVP complex and the ERSP portion is composed of about 3 to 60 amino acids. In some embodiments it is 5 to 50 amino acids. In some embodiments it is 10 to 40 amino acids but most often is composed of 15 to 20; 20 to 25; or 25 to 30 amino acids. The ERSP is a signal peptide so called because it directs the transportation of a protein. Signal peptides may also be called targeting signals, signal sequences, transit peptides, or localization signals. The signal peptides for ER trafficking are often 15 to 30 amino acid residues in length and have a tripartite organization, comprised of a core of hydrophobic residues flanked by a positively charged amino terminal and a polar, but uncharged carboxyterminal region. (Zimmermann, et al, “Protein translocation across the ER membrane”, Biochimica et Biohysica Acta, 2011, 1808: 912-924).

Many ERSPs are known. Many plant ERSPs are known. It is NOT required that the ERSP be derived from a plant ERSP, non-plant ERSPs will work with the procedures described herein. Many plant ERSPs are however well known and we describe some plant derived ERSPs here. BAAS, for example, is derived from the plant, Hordeum vulgare, and has the amino acid sequence as follows:

(SEQ ID NO: 5)
MANKHLSLSLFLVLLGLSASLASG

Plant ERSPs, which are selected from the genomic sequence for proteins that are known to be expressed and released into the apoplastic space of plants, include examples such as BAAS, carrot extensin, and tobacco PRI. The following references provide further descriptions, and are incorporated by reference herein in their entirety. De Loose, M. et al. “The extensin signal peptide allows secretion of a heterologous protein from protoplasts” Gene, 99 (1991) 95-100; De Loose, M. et al. described the structural analysis of an extension—encoding gene from Nicotiana plumbaginifolia, the sequence of which contains a typical signal peptide for translocation of the protein to the endoplasmic reticulum; Chen, M. H. et al. “Signal peptide-dependent targeting of a rice alpha-amylase and cargo proteins to plastids and extracellular compartments of plant cells” Plant Physiology, 2004 July; 135(3): 1367-77. Epub 2004 Jul 2. Chen, M. H. et al. studied the subcellular localization of α-amylases in plant cells by analyzing the expression of α-amylase, with and without its signal peptide, in transgenic tobacco. These references and others teach and disclose the signal peptide that can be used in the methods, procedures and peptide, protein and nucleotide complexes and constructs described herein.

The tobacco extensin signal peptide motif is an ERSP (Memelink et al, the Plant Journal, 1993, V4: 1011-1022; see also Pogue G P et al, Plant Biotechnology Journal, 2010, V8: 638-654). In some embodiments, an AVP expression ORF can have a tobacco extensin signal peptide motif. In one embodiment, the AVP expression ORF can have an extensin motif according to SEQ ID NO:12. In another embodiment, the AVP expression ORF can have an extensin motif according to SEQ ID NO:13. An illustrative example of how to generate an embodiment with an extensin signal motif is as follows: A DNA sequence encoding an extensin motif is designed (for example, the DNA sequence shown in SEQ ID NO:14 or SEQ ID NO: 15) using oligo extension PCR with four synthetic DNA primers; ends sites such as a restriction site, for example, a Pac I restriction site at the 5′-end, and a 5′-end of a GFP sequence at the 3′-end, can be added using PCR with the extensin DNA sequence serving as a template, and resulting in a fragment; the fragment is used as the forward PCR primer to amplify the DNA sequence encoding an AVP expression ORF, for example “gfy-l-avp” contained in a pFECT vector, thus producing an AVP expression ORF encoding (from N‘ to C’ terminal) “ERSP-GFP-L AVP” wherein the ERSP is extensin. The resulting DNA sequence can then be cloned into Pac I and Avr II restriction sites of a FECT vector to generate the pFECT-AVP vector for transient plant expression of GFP fused AVP.

In some embodiments, an illustrative expression system can include the FECT expression vectors containing AVP expression ORF is transformed into Agrobacterium, GV3101, and the transformed GV3101 is injected into tobacco leaves for transient expression of AVP expression ORF.

Translational stabilizing protein (STA) can increase the amount of AVP in plant tissues. One of the AVP expression ORF s, ERSP-AVP, is sufficient to express a properly folded AVP in the transfected plant, but in some embodiments, effective protection of a plant from pest damage may require that the plant expressed AVP accumulate. With transfection of a properly constructed AVP expression ORF, a transgenic plant can express and accumulate greater amounts of the correctly folded AVP. When a plant accumulates greater amounts of properly folded AVPs, it can more easily resist, inhibit, and/or kill the pests that attack and eat the plants. One method of increasing the accumulation of a polypeptide in transgenic tissues is through the use of a translational stabilizing protein (STA). The translational stabilizing protein can be used to significantly increase the accumulation of AVP in plant tissue, and thus increase the efficacy of a plant transfected with AVP with regard to pest resistance. The translational stabilizing protein is a protein with sufficient tertiary structure that it can accumulate in a cell without being targeted by the cellular process of protein degradation. The following equation describes one of the examples of an AVP expression ORF that encodes a stabilizing protein fused with Av3 variant polynucleotide sequence:
ersp-sta-l-avp

In some embodiments, the translational stabilizing protein can be a domain of another protein, or it can comprise an entire protein sequence. In some embodiments, the translational stabilizing protein can be between 5 and 50 amino acids, 50 to 250 amino acids (e.g., GNA), 250 to 750 amino acids (e.g., chitinase) and 750 to 1500 amino acids (e.g., enhancin).

The protein, or protein domain can contain proteins that have no useful characteristics other than translation stabilization, or they can have other useful traits in addition to translational stabilization. Useful traits can include: additional insecticidal activity, such as activity that is destructive to the peritrophic membrane, activity that is destructive to the gut wall, and/or activity that actively transports the AVP across the gut wall. One embodiment of the translational stabilizing protein can be a polymer of fusion proteins involving AVP. A specific example of a translational stabilizing protein is provided here to illustrate the use of a translational stabilizing protein. The example is not intended to limit the disclosure or claims in any way. Useful translational stabilizing proteins are well known in the art, and any proteins of this type could be used as disclosed herein. Procedures for evaluating and testing production of peptides are both known in the art and described herein. One example of one translational stabilizing protein is Green-Fluorescent Protein (GFP) (SEQ ID NO:9; NCBI Accession No. AAF65230.1).

Additional examples of translational stabilizing proteins can be found in the following references, the disclosures of which are incorporated by reference in their entirety: Kramer, K. J. et al. “Sequence of a cDNA and expression of the gene encoding epidermal and gut chitinases of Manduca sexta” Insect Biochemistry and Molecular Biology, Vol. 23, Issue 6, September 1993, pp. 691-701. Kramer, K. J. et al. isolated and sequenced a chitinase-encoding cDNA from the tobacco hornworm, Manduca sexta. Hashimoto, Y. et al. “Location and nucleotide sequence of the gene encoding the viral enhancing factor of the Trichoplusia ni granulosis virus” Journal of General Virology, (1991), 72, 2645-2651. Hashimoto, Y. et al. cloned the gene encoding the viral enhancing factor of a Trichoplusia ni granulosis virus and determined the complete nucleotide sequence. Van Damme, E. J. M. et al. “Biosynthesis, primary structure and molecular cloning of snowdrop (Galanthus nivalis L.) lectin” European Journal of Biochemistry, 202, 23-30 (1991). Van Damme, E. J. M. et al. isolated Poly(A)-rich RNA from ripening ovaries of snowdrop lectin (GNA), yielding a single 17-kDa lectin polypeptide upon translation in a wheat-germ cell-free system, called agglutin. These references and others teach and disclose translational stabilizing proteins that can be used in the methods, procedures and peptide, protein and nucleotide complexes and constructs described herein.

In some embodiments, an AVP expression ORF can be transformed into a plant, for example, in the tobacco plant, Nicotiana benthamiana, using an AVP expression ORF that contains a STA, for example Jun a 3. The mature Jun a 3 is a ˜30 kDa plant defending protein that is also an allergen for some people. Jun a 3 is produced by Juniperus ashei trees and can be used in some embodiments as a translational stabilizing protein (STA). In some embodiments, the Jun a 3 amino acid sequence can be the sequence shown in SEQ ID NO:16. In other embodiments, the Jun a 3 amino acid sequence can be the sequence shown in SEQ ID NO:11.

In some embodiments, an AVP expression ORF can contain an STA, for example, snowdrop lectin (GNA) having the sequence shown in either SEQ ID NO:10 or SEQ ID NO:17.

Linker proteins assist in the proper folding of the different motifs composing an AVP expression ORF. The AVP expression ORF described in this invention also incorporates polynucleotide sequences encoding intervening linker peptides between the polynucleotide sequences encoding the AVP (avp) and the translational stabilizing protein (sta), or between polynucleotide sequence encoding multiple polynucleotide sequences encoding AVP, i.e., (l-avp)_{N} or (avp-l)_{N}, if the expression ORF involves multiple AVP domain expression. The intervening linker peptides (LINKERS or L) separate the different parts of the expressed AVP complex and help proper folding of the different parts of the complex during the expression process. In the expressed AVP complex, different intervening linker peptides can be involved to separate different functional domains. In some embodiments, the LINKER is attached to a AVP and this bivalent group can be repeated up to 10 (N=1-10) and possibly even more than 10 times in order to facilitate the accumulation of properly folded AVP in the plant that is to be protected.

In some embodiments the intervening linker peptide can be between 1 and 30 amino acids in length. However, it is not necessarily an essential component in the expressed AVP in plants. A cleavable linker peptide can be designed to the AVP expression ORF to release the properly AVP from the expressed AVP complex in the transformed plant to improve the protection the AVP affords the plant with regard to pest damage. One type of the intervening linker peptide is the plant cleavable linker peptide. This type of linker peptides can be completely removed from the expressed AVP expression ORF complex during plant post-translational modification. Therefore, in some embodiments, the properly folded AVP linked by this type of intervening linker peptides can be released in the plant cells from the expressed AVP expression ORF complex during post-translational modification in the plant.

Another type of the cleavable intervening linker peptide is not cleavable during the expression process in plants. However, it has a protease cleavage site specific to serine, threonine, cysteine, aspartate proteases or metalloproteases. The type of cleavable linker peptide can be digested by proteases found in the insect and lepidopteran gut environment and/or the insect hemolymph and lepidopteran hemolymph environment to release the AVP in the insect gut or hemolymph. Using the information taught by this disclosure it should be a matter of routine for one skilled in the art to make or find other examples of LINKERS that will be useful in this invention.

In some embodiments, the AVP expression ORF can contain a cleavable type of intervening linker, for example, the type listed in SEQ ID NO:6, having the amino acid code of “IGER” (SEQ ID NO:6). The molecular weight of this intervening linker or LINKER is 473.53 Daltons. In other embodiments, the intervening linker peptide (LINKER) can also be one without any type of protease cleavage site, i.e. an uncleavable intervening linker peptide, for example, the linker “ETMFKHGL” (SEQ ID NO:8).

Other examples of intervening linker peptides can be found in the following references, which are incorporated by reference herein in their entirety: A plant expressed serine proteinase inhibitor precursor was found to contain five homogeneous protein inhibitors separated by six same linker peptides in Heath et al. “Characterization of the protease processing sites in a multidomain proteinase inhibitor precursor from Nicotiana alata” European Journal of Biochemistry, 1995; 230: 250-257. A comparison of the folding behavior of green fluorescent proteins through six different linkers is explored in Chang, H. C. et al. “De novo folding of GFP fusion proteins: high efficiency in eukaryotes but not in bacteria” Journal of Molecular Biology, 2005 Oct 21; 353(2): 397-409. An isoform of the human GalNAc-Ts family, GalNAc-T2, was shown to retain its localization and functionality upon expression in N. benthamiana plants by Daskalova, S. M. et al. “Engineering of N. benthamiana L. plants for production of N-acetylgalactosamine-glycosylated proteins” BMC Biotechnology, 2010 Aug. 24; 10: 62. The ability of endogenous plastid proteins to travel through stromules was shown in Kwok, E. Y. et al. “GFP-labelled Rubisco and aspartate aminotransferase are present in plastid stromules and traffic between plastids” Journal of Experimental Botany, 2004 March; 55(397): 595-604. Epub 2004 Jan. 30. A report on the engineering of the surface of the tobacco mosaic virus (TMV), virion, with a mosquito decapeptide hormone, trypsin-modulating oostatic factor (TMOF) was made by Borovsky, D. et al. “Expression of Aedes trypsin-modulating oostatic factor on the virion of TMV: A potential larvicide” Proc Natl Acad Sci, 2006 Dec. 12; 103(50): 18963-18968. These references and others teach and disclose the intervening linkers that can be used in the methods, procedures and peptide, protein and nucleotide complexes and constructs described herein.

The AVP expression ORF described above can be cloned into any plant expression vector for AVP to be expression in plants, either transiently or stably.

Transient plant expression systems can be used to promptly optimize the structure of the AVP expression ORF for some specific AVP expression in plants, including the necessity of some components, codon optimization of some components, optimization of the order of each component, etc. A transient plant expression vector is often derived from a plant virus genome. Plant virus vectors provide advantages in quick and high level of foreign gene expression in plant due to the infection nature of plant viruses. The full length of the plant viral genome can be used as a vector, but often a viral component is deleted, for example the coat protein, and transgenic ORFs are subcloned in that place. The AVP expression ORF can be subcloned into such a site to create a viral vector. These viral vectors can be introduced into plant mechanically since they are infectious themselves, for example through plant wound, spray-on etc. They can also be transfected into plants via agroinfection, by cloning the virus vector into the T-DNA of the crown gall bacterium, Agrobacterium tumefaciens, or the hairy root bacterium, Agrobacterium rhizogenes. The expression of the AVP in this vector is controlled by the replication of the RNA virus, and the virus translation to mRNA for replication is controlled by a strong viral promoter, for example, 35S promoter from Cauliflower mosaic virus. Viral vectors with AVP expression ORF are usually cloned into T-DNA region in a binary vector that can replicate itself in both E. coli strains and Agrobacterium strains. The transient transfection of a plant can be done by infiltration of the plant leaves with the Agrobacterium cells which contain the viral vector for AVP expression. In the transient transformed plant, it is common for the foreign protein expression to be ceased in a short period of time due to the post-transcriptional gene silencing (PTGS). Sometimes a PTGS suppressing protein gene is necessary to be co-transformed into the plant transiently with the same type of viral vector that drives the expression of with the AVP expression ORF. This improves and extends the expression of the AVP in the plant. The most commonly used PTGS suppressing protein is P19 protein discovered from tomato bushy stunt virus (TBSV).

In some embodiments, transient transfection of plants can be achieved by recombining a polynucleotide encoding an AVP with any one of the readily available vectors (see above), and confirmed, using a marker or signal (e.g., GFP emission) In some embodiments, a transiently transfected plant can be created by recombining a polynucleotide encoding an AVP with a DNA encoding a GFP-Hybrid fusion protein in a vector, and transfection said vector into a plant (e.g., tobacco) using different FECT vectors designed for targeted expression. In some embodiments, a polynucleotide encoding an AVP can be recombined with a pFECT vector for APO (apoplast localization) accumulation; a pFECT vector for CYTO (cytoplasm localization) accumulation; or pFECT with ersp vector for ER (endoplasm reticulum localization) accumulation.

An exemplary transient plant transformation strategy is agroinfection using a plant viral vector due to its high efficiency, ease, and low cost. In some embodiments, a tobacco mosaic virus overexpression system (see TRBO, Lindbo J A, Plant Physiology, 2007, V145: 1232-1240) can be used to transiently transform plants with AVP. The TRBO DNA vector has a T-DNA region for agroinfection, which contains a CaMV 35S promoter that drives expression of the tobacco mosaic virus RNA without the gene encoding the viral coating protein. Moreover, this system uses the “disarmed” virus genome, therefore viral plant to plant transmission can be effectively prevented.

In another embodiment, the FECT viral transient plant expression system can be used to transiently transform plants with AVP (see Liu Z & Kearney CM, BMC Biotechnology, 2010, 10:88). The FECT vector contains a T-DNA region for agroinfection, which contains a CaMV 35S promoter that drives the expression of the foxtail mosaic virus RNA without the genes encoding the viral coating protein and the triple gene block. Moreover, this system uses the “disarmed” virus genome, therefore viral plant to plant transmission can be effectively prevented. To efficiently express the introduced heterologous gene, the FECT expression system additionally needs to co-express P19, a RNA silencing suppressor protein from tomato bushy stunt virus, to prevent the post-transcriptional gene silencing (PTGS) of the introduced T-DNA. (The TRBO expression system does not need co-expression of P19).

In some embodiments, the AVP expression ORF can be designed to encode a series of translationally fused structural motifs that can be described as follows: N′-ERSP-STA-L-AVP-C′ wherein the “N′” and “C′” indicating the N-terminal and C-terminal amino acids, respectively, and the ERSP motif can be the Barley Alpha-Amylase Signal peptide (BAAS) (SEQ ID NO:5); the stabilizing protein (STA) can be GFP (SEQ ID NO:9); the linker peptide “L” can be IGER (SEQ ID NO:6) In some embodiments, the ersp-sta-l-avp ORF can chemically synthesized to include restrictions sites, for example a Pac I restriction site at its 5′-end, and an Avr II restriction site at its 3′-end. In some embodiments, the AVP expression ORF can be cloned into the Pac I and Avr II restriction sites of a FECT expression vector (pFECT) to create an Av3 variant expression vector for the FECT transient plant expression system (pFECT-AVP). To maximize expression in the FECT expression system, some embodiments may have a FECT vector expressing the RNA silencing suppressor protein P19 (pFECT-P19) generated for co-transformation.

In some embodiments, an Av3 variant expression vector can be recombined for use in a TRBO transient plant expression system, for example, by performing a routine PCR procedure and adding a Not I restriction site to the 3′-end of the AVP expression ORF described above, and then cloning the AVP expression ORF into Pac I and Not I restriction sites of the TRBO expression vector (pTRBO-AVP).

In some embodiments, an Agrobacterium tumefaciens strain, for example, commercially available GV3101 cells, can be used for the transient expression of an AVP expression ORF in a plant tissue (e.g., tobacco leaves) using one or more transient expression systems, for example, the FECT and TRBO expression systems. An exemplary illustration of such a transient transfection protocol includes the following: an overnight culture of GV3101 can be used to inoculate 200 mL Luria-Bertani (LB) medium; the cells can be allowed to grow to log phase with OD600 between 0.5 and 0.8; the cells can then be pelleted by centrifugation at 5000 rpm for 10 minutes at 4° C.; cells can then be washed once with 10 mL prechilled TE buffer (Tris-HCl 10 mM, EDTA 1 mM, pH8.0), and then resuspended into 20 mL LB medium; GV3101 cell resuspension can then be aliquoted in 250 μL fractions into 1.5 mL microtubes; aliquots can then be snap-frozen in liquid nitrogen and stored at −80° C. freezer for future transformation. The pFECT-AVP and pTRBO-AVP vectors can then transformed into the competent GV3101 cells using a freeze-thaw method as follows: the stored competent GV3101 cells are thawed on ice and mixed with 1 to 5 μg pure DNA (pFECT-AVP or pTRBO-AVP vector). The cell-DNA mixture is kept on ice for 5 minutes, transferred to −80° C. for 5 minutes, and incubated in a 37° C. water bath for 5 minutes. The freeze-thaw treated cells are then diluted into 1 mL LB medium and shaken on a rocking table for 2 to 4 hours at room temperature. A 200 μL aliquot of the cell-DNA mixture is then spread onto LB agar plates with the appropriate antibiotics (10 μg/mL rifampicin, 25 μg/mL gentamycin, and 50 μg/mL kanamycin can be used for both pFECT-AVP transformation and pTRBO-AVP transformation) and incubated at 28° C. for two days. Resulting transformed colonies are then picked and cultured in 6 mL aliquots of LB medium with the appropriate antibiotics for transformed DNA analysis and making glycerol stocks of the transformed GV3101 cells.

In some embodiments, the transient transformation of plant tissues, for example, tobacco leaves, can be performed using leaf injection with a 3-mL syringe without needle. In one illustrative example, the transformed GV3101 cells are streaked onto an LB plate with the appropriate antibiotics (as described above) and incubated at 28° C. for two days. A colony of transformed GV3101 cells are inoculated to 5 ml of LB-MESA medium (LB media supplemented with 10 mM MES, and 20 μM acetosyringone) and the same antibiotics described above, and grown overnight at 28° C. The cells of the overnight culture are collected by centrifugation at 5000 rpm for 10 minutes and resuspended in the induction medium (10 mM MES, 10 mM MgCl₂, 100 μM acetosyringone) at a final OD600 of 1.0. The cells are then incubated in the induction medium for 2 hours to overnight at room temperature and are then ready for transient transformation of tobacco leaves. The treated cells can be infiltrated into the underside of attached leaves of Nicotiana benthamiana plants by injection, using a 3-mL syringe without a needle attached.

In some embodiments, the transient transformation can be accomplished by transfecting one population of GV3101 cells with pFECT-AVP or pTRBO-AVP and another population with pFECT-P19, mixing the two cell populations together in equal amounts for infiltration of tobacco leaves by injection with a 3-mL syringe.

Stable integration of polynucleotide operable to encode AVP is also possible with the present disclosure, for example, the AVP expression ORF can also be integrated into plant genome using stable plant transformation technology, and therefore AVPs can be stably expressed in plants and protect the transformed plants from generation to generation. For the stable transformation of plants, the AVPs expression vector can be circular or linear. The AVP expression ORF, the AVP expression cassette, and/or the vector with polynucleotide encoding an AVP for stable plant transformation should be carefully designed for optimal expression in plants based on what is known to those having ordinary skill in the art, and/or by using predictive vector design tools such as Gene Designer 2.0 (Atum Bio); VectorBuilder (Cyagen); SnapGene® viewer; GeneArt™ Plasmid Construction Service (Thermo-Fisher Scientific); and/or other commercially available plasmid design services. See Tolmachov, Designing plasmid vectors. Methods Mol Biol. 2009; 542:117-29. The expression of AVP is usually controlled by a promoter that promotes transcription in some, or all the cells of the transgenic plant. The promoter can be a strong plant viral promoter, for example, the constitutive 35S promoter from Cauliflower Mosaic Virus (CaMV); it also can be a strong plant promoter, for example, the hydroperoxide lyase promoter (pHPL) from Arabidopsis thaliana; the Glycine max polyubiquitin (Gmubi) promoter from soybean; the ubiquitin promoters from different plant species (rice, corn, potato, etc.), etc. A plant transcriptional terminator often occurs after the stop codon of the ORF to halt the RNA polymerase and transcription of the mRNA. To evaluate the AVPs expression, a reporter gene can be included in the AVPs expression vector, for example, beta-glucuronidase gene (GUS) for GUS straining assay, green fluorescent protein (GFP) gene for green fluorescence detection under UV light, etc. For selection of transformed plants, a selection marker gene is usually included in the AVP expression vector. In some embodiments, the marker gene expression product can provide the transformed plant with resistance to specific antibiotics, for example, kanamycin, hygromycin, etc., or specific herbicide, for example, glyphosate etc. If agroinfection technology is adopted for plant transformation, T-DNA left border and right border sequences are also included in the AVPs expression vector to transport the T-DNA portion into the plant.

The constructed AVPs expression vector can be transfected into plant cells or tissues using many transfection technologies. Agroinfection is a very popular way to transform a plant using an Agrobacterium tumefaciens strain or an Agrobacterium rhizogenes strain. Particle bombardment (also called Gene Gun, or Biolistics) technology is also very common method of plant transfection. Other less common transfection methods include tissue electroporation, silicon carbide whiskers, direct injection of DNA, etc. After transfection, the transfected plant cells or tissues placed on plant regeneration media to regenerate successfully transfected plant cells or tissues into transgenic plants.

Evaluation of a transformed plant can be accomplished at the DNA level, RNA level and protein level. A stably transformed plant can be evaluated at all of these levels and a transiently transformed plant is usually only evaluated at protein level. To ensure that the AVP expression ORF integrates into the genome of a stably transformed plant, the genomic DNA can be extracted from the stably transformed plant tissues for and analyzed using PCR or Southern blot. The expression of the AVP in the stably transformed plant can be evaluated at the RNA level, for example, by analyzing total mRNA extracted from the transformed plant tissues using northern blot or RT-PCR. The expression of the AVP in the transformed plant can also be evaluated in protein level directly. There are many ways to evaluate expression of AVP in a transformed plant. If a reporter gene included in the AVP expression ORF, a reporter gene assay can be performed, for example, in some embodiments a GUS straining assay for GUS reporter gene expression, a green fluorescence detection assay for GFP reporter gene expression, a luciferase assay for luciferase reporter gene expression, and/or other reporter techniques may be employed.

In some embodiments total protein can be extracted from the transformed plant tissues for the direct evaluation of the expression of the AVP using a Bradford assay to evaluate the total protein level in the sample.

In some embodiments, analytical HPLC chromatography technology, Western blot technique, or iELISA assay can be adopted to qualitatively or quantitatively evaluate the AVP in the extracted total protein sample from the transformed plant tissues. AVP expression can also be evaluated by using the extracted total protein sample from the transformed plant tissues in an insect bioassay, for example, in some embodiments, the transformed plant tissue or the whole transformed plant itself can be used in insect bioassays to evaluate AVP expression and its ability to provide protection for the plant.

Confirming Successful Transformation with AVP

Following introduction of heterologous foreign DNA into plant cells, the transformation or integration of heterologous gene in the plant genome is confirmed by various methods such as analysis of nucleic acids, proteins and metabolites associated with the integrated gene.

PCR analysis is a rapid method to screen transformed cells, tissue or shoots for the presence of incorporated gene at the earlier stage before transplanting into the soil (Sambrook and Russell (2001) Molecular Cloning: A Laboratory Manual. Cold Spring Harbor Laboratory Press, Cold Spring Harbor, N.Y.). PCR is carried out using oligonucleotide primers specific to the gene of interest or Agrobacterium vector background, etc.

Plant transformation may be confirmed by Southern blot analysis of genomic DNA (Sambrook and Russell, 2001, supra). In general, total DNA is extracted from the transformed plant, digested with appropriate restriction enzymes, fractionated in an agarose gel and transferred to a nitrocellulose or nylon membrane. The membrane or “blot” is then probed with, for example, radiolabeled ³²P target DNA fragment to confirm the integration of introduced gene into the plant genome according to standard techniques (Sambrook and Russell, 2001, supra).

In Northern blot analysis, RNA is isolated from specific tissues of transformed plant, fractionated in a formaldehyde agarose gel, and blotted onto a nylon filter according to standard procedures that are routinely used in the art (Sambrook and Russell, 2001, supra). Expression of RNA encoded by the polynucleotide encoding an AVP is then tested by hybridizing the filter to a radioactive probe derived from an AVP, by methods known in the art (Sambrook and Russell, 2001, supra).

Western blot, biochemical assays and the like may be carried out on the transgenic plants to confirm the presence of protein encoded by the AVP gene by standard procedures (Sambrook and Russell, 2001, supra) using antibodies that bind to one or more epitopes present on the AVP.

A number of markers have been developed to determine the success of plant transformation, for example, resistance to chloramphenicol, the aminoglycoside G418, hygromycin, or the like. Other genes that encode a product involved in chloroplast metabolism may also be used as selectable markers. For example, genes that provide resistance to plant herbicides such as glyphosate, bromoxynil, or imidazolinone may find particular use. Such genes have been reported (Stalker et al. (1985) J. Biol. Chem. 263:6310-6314 (bromoxynil resistance nitrilase gene); and Sathasivan et al. (1990) Nucl. Acids Res. 18:2188 (AHAS imidazolinone resistance gene). Additionally, the genes disclosed herein are useful as markers to assess transformation of bacterial, yeast, or plant cells. Methods for detecting the presence of a transgene in a plant, plant organ (e.g., leaves, stems, roots, etc.), seed, plant cell, propagule, embryo or progeny of the same are well known in the art. In one embodiment, the presence of the transgene is detected by testing for pesticidal activity.

Fertile plants expressing an AVP and/or Av3 variant polynucleotide may be tested for pesticidal activity, and the plants showing optimal activity selected for further breeding. Methods are available in the art to assay for pest activity. Generally, the protein is mixed and used in feeding assays. See, for example Marrone et al. (1985) J. of Economic Entomology 78:290-293.

In some embodiments, evaluating the success of a transient transfection procedure can be determined based on the expression of a reporter gene, for example, GFP. In some embodiments, GFP can be detected under U.V. light in tobacco leaves transformed with the FECT and/or TRBO vectors.

In some embodiments, AVP expression can be quantitatively evaluated in a plant (e.g., tobacco). An exemplary procedure that illustrates AVP quantification in a tobacco plant is as follows: 100 mg disks of transformed leaf tissue is collected by punching leaves with the large opening of a 1000 μL pipette tip. The collected leaf tissue is place into a 2 mL microtube with 5/32″ diameter stainless steel grinding balls, and frozen in −80° C. for 1 hour, and then homogenized using a Troemner-Talboys High Throughput Homogenizer. Next, 750 μL ice-cold TSP-SE1 extraction solutions (sodium phosphate solution 50 mM, 1:100 diluted protease inhibitor cocktail, EDTA 1 mM, DIECA 10 mM, PVPP 8%, pH 7.0) is added into the tube and vortexed. The microtube is then left still at room temperature for 15 minutes and then centrifuged at 16,000 g for 15 minutes at 4° C.; 100 μL of the resulting supernatant is taken and loaded into pre-Sephadex G-50-packed column in 0.45 μm Millipore MultiScreen filter microtiter plate with empty receiving Costar microtiter plate on bottom. The microtiter plates are then centrifuged at 800 g for 2 minutes at 4° C. The resulting filtrate solution, herein called total soluble protein extract (TSP extract) of the tobacco leaves, is then ready for the quantitative analysis.

In some embodiments, the total soluble protein concentration of the TSP extract can be estimated using Pierce Coomassie Plus protein assay. BSA protein standards with known concentrations can be used to generate a protein quantification standard curve. For example, 2 μL of each TSP extract can be mixed into 200 μL of the chromogenic reagent (CPPA reagent) of the Coomassie Plus protein assay kits and incubated for 10 minutes. The chromogenic reaction can then be evaluated by reading OD595 using a SpectroMax-M2 plate reader using SoftMax Pro as control software. The concentrations of total soluble proteins can be about 0.788±0.20 μg/μL or about 0.533±0.03 μg/μL in the TSP extract from plants transformed via FECT and TRBO, respectively, and the results can be used to calculate the percentage of the expressed U-Av3 variant peptide in the TSP (% TSP) for the iELISA assay.

In some embodiments, an indirect ELISA (iELISA) assay can be used to quantitatively evaluate the AVP content in the tobacco leaves transiently transformed with the FECT and/or TRBO expression systems. An illustrative example of using iELISA to quantify AVP is as follows: 5 μL of the leaf TSP extract is diluted with 95 μL of CB2 solution (Immunochemistry Technologies) in the well of an Immulon 2HD 96-well plate, with serial dilutions performed as necessary; leaf proteins obtained from extract samples are then allowed to coat the well walls for 3 hours in the dark, at room temperature, and the CB2 solution is then subsequently removed; each well is washed twice with 200 μL PBS (Gibco); 150 μL blocking solution (Block BSA in PBS with 5% non-fat dry milk) is added into each well and incubated for 1 hour, in the dark, at room temperature; after the removal of the blocking solution, a PBS wash of the wells, 100 μL of primary antibodies directed against AVP (custom antibodies are commercially available from ProMab Biotechnologies, Inc.; GenScript®; or raised using the knowledge readily available to those having ordinary skill in the art); the antibodies diluted at 1:250 dilution in blocking solution are added to each well and incubated for 1 hour in the dark at room temperature; the primary antibody is removed and each well is washed with PBS 4 times; 100 μL of HRP-conjugated secondary antibody (i.e., antibody directed against host species used to generate primary antibody, used at 1: 1000 dilution in the blocking solution) is added into each well and incubated for 1 hour in the dark at room temperature; the secondary antibody is removed and the wells are washed with PBS, 100 μL; substrate solution (a 1:1 mixture of ABTS peroxidase substrate solution A and solution B, KPL) is added to each well, and the chromogenic reaction proceeds until sufficient color development is apparent; 100 μL of peroxidase stop solution is added to each well to stop the reaction; light absorbance of each reaction mixture in the plate is read at 405 nm using a SpectroMax-M2 plate reader, with SoftMax Pro used as control software; serially diluted known concentrations of pure AVPs samples can be treated in the same manner as described above in the iELISA assay to generate a mass-absorbance standard curve for quantities analysis. The expressed AVP can be detected by iELISA at about 3.09±1.83 ng/μL in the leaf TSP extracts from the FECT transformed tobacco; and about 3.56±0.74 ng/μL in the leaf TSP extract from the TRBO transformed tobacco. Alternatively, the expressed AVP can be about 0.40% total soluble protein (% TSP) for FECT transformed plants and about 0.67% TSP in TRBO transformed plants.

### Mixtures, Products, and Transgenic Organisms Utilizing AVP.

Any of the mixtures, products, polypeptides and/or plants utilizing AVP, and described herein, can be used to control pests, their growth, and/or the damage caused by their actions, especially their damage to plants. Compositions comprising AVP, for example, agrochemical compositions, can include, but is not limited to, aerosols and/or aerosolized products, for example, sprays, fumigants, powders, dusts, and/or gases; seed dressings; oral preparations (e.g., insect food, etc.); transgenic organisms expressing and/or producing AVP and/or an AVP expression ORF (either transiently and/or stably), for example, a plant or an animal. In some embodiments, compositions comprising an AVP or an insecticidal protein comprising one or more AVPs and one or more non-AVP peptides, polypeptides and proteins can be used concomitantly, or sequentially with other insecticides proteins, and/or pesticides as described in Table 1, for example, the Bt toxin, AaIT1, Bti, pymethrin, and other known insecticides for example, Na⁺ channel agonists (i.e., pyrethroids), Na⁺ channel blocking agents (i.e., pyrazolines), acetylcholinesterase inhibitors (i.e., organophosphates and carbamates), nicotinic acetylcholine binding agents (e.g., imidacloprid), gabaergic binding agents (e.g., emamectin and fipronil), octapamine agonists or antagonists (i.e., formamidines), and oxphos uncouplers (e.g., pyrrole insecticides).

In some embodiments, the active ingredients of the present disclosure can be applied in the form of compositions and can be applied to the crop area or plant to be treated, simultaneously or in succession, with other compounds. These compounds can be fertilizers, weed killers, cryoprotectants, surfactants, detergents, pesticidal soaps, dormant oils, polymers, and/or time-release or biodegradable carrier formulations that permit long-term dosing of a target area following a single application of the formulation. They can also be selective herbicides, chemical insecticides, virucides, microbicides, amoebicides, pesticides, fungicides, bacteriocides, nematocides, molluscicides or mixtures of several of these preparations, if desired, together with further agriculturally acceptable carriers, surfactants or application-promoting adjuvants customarily employed in the art of formulation. Suitable carriers and adjuvants can be solid or liquid and correspond to the substances ordinarily employed in formulation technology, e.g. natural or regenerated mineral substances, solvents, dispersants, wetting agents, tackifiers, binders or fertilizers. Likewise, the formulations may be prepared into edible “baits” or fashioned into pest “traps” to permit feeding or ingestion by a target pest of the pesticidal formulation.

Methods of applying an active ingredient of the present disclosure or an agrochemical composition of the present disclosure that contains at least one of the AVPs produced by the methods described herein of the present disclosure include leaf application, seed coating and soil application. In some embodiments, the number of applications and the rate of application depend on the intensity of infestation by the corresponding pest.

The composition may be formulated as a powder, dust, pellet, granule, spray, emulsion, colloid, solution, or such like, and may be prepared by such conventional means as desiccation, lyophilization, homogenization, extraction, filtration, centrifugation, sedimentation, or concentration of a culture of cells comprising the polypeptide. In all such compositions that contain at least one such pesticidal polypeptide, the polypeptide may be present in a concentration of from about 1% to about 99% by weight.

In some embodiments, compositions containing AVPs may be prophylactically applied to an environmental area to prevent infestation by a susceptible pest, for example, a lepidopteran and/or coleopteran pest, which may be killed or reduced in numbers in a given area by the methods of the invention. In some embodiments, the pest ingests, or comes into contact with, a pesticidally-effective amount of the polypeptide.

In some embodiments, the pesticide compositions described herein may be made by formulating either the bacterial, yeast, or other cell, crystal and/or spore suspension, or isolated protein component with the desired agriculturally-acceptable carrier. The compositions may be formulated prior to administration in an appropriate means such as lyophilized, freeze-dried, desiccated, or in an aqueous carrier, medium or suitable diluent, such as saline and/or other buffer. In some embodiments, the formulated compositions may be in the form of a dust or granular material, or a suspension in oil (vegetable or mineral), or water or oil/water emulsions, or as a wettable powder, or in combination with any other carrier material suitable for agricultural application. Suitable agricultural carriers can be solid or liquid and are well known in the art. In some embodiments, the formulations may be mixed with one or more solid or liquid adjuvants and prepared by various means, e.g., by homogeneously mixing, blending and/or grinding the pesticidal composition with suitable adjuvants using conventional formulation techniques. Suitable formulations and application methods are described in U.S. Pat. No. 6,468,523, herein incorporated by reference.

### Compositions Comprising AVP and Other Products

In some embodiments, a composition comprising AVP may also comprise additional ingredients, for example, herbicides, chemical insecticides, virucides, microbicides, amoebicides, pesticides, fungicides, bacteriocides, nematocides, molluscicides, polypeptides, and/or one or more of the foregoing mixtures thereof. For example, in some embodiments, a composition comprising AVP may also contain one or more polypeptides, for example, AaIT1 (sodium channel gating modifier from the scorpion, Androctonus australis hector), and/or Bt (a toxin derived from Bacillus thuringiensis, for example, Bti derived from Bacillus thuringiensis serotype israelensis).

Bt are the initials for a bacterium called Bacillus thuringiensis. The Bt bacteria produces a family of peptides that are toxic to many insects. The Bt toxic peptides are well known for their ability to produce parasporal crystalline protein inclusions (usually referred to as crystals) that fall under two major classes of toxins; cytolysins (Cyt) and crystal Bt proteins (Cry). Since the cloning and sequencing of the fSSIt crystal proteins genes in the early-1980s, may others have been characterized and are now classified according to the nomenclature of Crickmore et al. (1998). Generally, Cyt proteins are toxic towards the insect orders Coleoptera (beetles) and Diptera (flies), and Cry proteins target Lepidopterans (moths and butterflies). Cry proteins bind to specific receptors on the membranes of mid-gut (epithelial) cells resulting in rupture of those cells. If a Cry protein cannot find a specific receptor on the epithelial cell to which it can bind, then it is not toxic. Bt strains can have different complements of Cyt and Cry proteins, thus defining their host ranges. The genes encoding many Cry proteins have been identified.

Currently there are four main pathotypes of insecticidal Bt parasporal peptides based on order specificity: Lepidotera-specific (CryI, now Cry1), Coleoptera-specific (CryIII, now Cry3), Diptera-specific (CryIV, now Cry4, Cry 10, Cry11; and CytA, now Cyt1A), and CryII (Now Cry2), the only family known at that time to have dual (Lepidoptera and Diptera) specificity. Cross-order activity is now apparent in many cases.

The nomenclature assigns holotype sequences a unique name which incorporates ranks based on the degree of divergence, with the boundaries between the primary (Arabic numeral), secondary (uppercase letter), and tertiary (lower case letter) rank representing approximately 95%, 78% and 45% identities. A fourth rank (another Arabic number) is used to indicate independent isolations of holotype toxin genes with sequences that are identical or differ only slightly. Currently, the nomenclature distinguishes 174 holotype sequences that are grouping in 55 cry and 2 cyt families (Crickmore, N., Zeigler, D. R., Schnepf, E., Van Rie, J., Lereclus, D., Daum, J, Bravo, A., Dean, D. H., B. thuringiensis toxin nomenclature). Any of these crystal proteins and the genes that produce them may be used to produce a suitable Bt related toxin for this invention.

Also included in the descriptions of this invention are families of highly related crystal proteins produced by other bacteria: Cry16 and Cry17 from Clostridium bifermentans (Barloy et al., 1996, 1998), Cry 18 from Bacillus popilliae (Zhang et al., 1997), Cry43 from Paenibacillus lentimorbis (Yokoyama et al., 2004) and the binary Cry48/Cry49 produced by Bacillus sphaericus (Jones et al., 2008). Other crystalline or secreted pesticidal proteins, such as the S-layer proteins (Peia et al., 2006) that are included here are, genetically altered crystal proteins, except those that were modified through single amino acid substitutions (e.g., Lambert et al., 1996). Any of these genes may be used to produce a suitable Bt related toxin for this invention.

Naturally occurring allelic variants can be identified with the use of well-known molecular biology techniques, such as polymerase chain reaction (PCR) and hybridization techniques as outlined below. Variant nucleotide sequences also include synthetically derived nucleotide sequences that have been generated, for example, by using site-directed mutagenesis but which still encode the Bt protein proteins disclosed in the present disclosure as discussed below. Variant proteins encompassed by the present disclosure are biologically active, that is they continue to possess the desired biological activity of the native protein, i.e., retaining pesticidal activity. By “retains activity” is intended that the variant will have at least about 30%, at least about 50%, at least about 70%, or at least about 80% of the pesticidal activity of the native protein. Methods for measuring pesticidal activity are well known in the art. See, for example, Czapla and Lang (1990) J. Econ. Entomol. 83: 2480-2485; Andrews et al. (1988) Biochem. J. 252:199-206; Marrone et al. (1985) J. of Economic Entomology 78:290-293; and U.S. Pat. No. 5,743,477, all of which are herein incorporated by reference in their entirety, and all sequences identified by number specifically incorporated by reference.

Bt proteins and gene descriptions are described below in the following the following tables, which contain the Bt toxin and corresponding reference, each of which is incorporated by reference in its entirety.

TABLE 2
Bt Toxins and References.

| Toxin | Patents or Patent Publication Number |
|---|---|
| Crv1 | US2003046726. U.S. Pat. No. 6,833,449. |
| | CN1260397. US201026939. |
| | US2006174372. US2006174372. |
| | US642241. U.S. Pat. No. 6,229,004. |
| | US2004194165. U.S. Pat. No. 6,573,240. |
| | U.S. Pat. No. 5,424,409. U.S. Pat. No. 5,407,825. |
| | U.S. Pat. No. 5,135,867. U.S. Pat. No. 5,055,294. |
| Crv1 | WO2007107302. U.S. Pat. No. 6,855,873. |
| | WO2004020636. US2007061919. |
| | U.S. Pat. No. 6,048,839. US2007061919. |
| | AU784649B. US2007061919. U.S. Pat. No. 6,150,589. |
| | U.S. Pat. No. 5,679,343. U.S. Pat. No. 5,616,319. |
| | U.S. Pat. No. 5,322,687. |
| Crv1 | WO2007107302. US2006174372. |
| | US2005091714. US2004058860. US2008020968. |
| | U.S. Pat. No. 6,043,415. U.S. Pat. No. 5,942,664. |
| Crv1 | WO2007107302. US2007061919. |
| | U.S. Pat. No. 6,172,281. |
| Crv1 | WO03082910. MX9606262. U.S. Pat. No. 5,530,195. |
| | U.S. Pat. No. 5,407,825. U.S. Pat. No. 5,045,469. |
| Crv1 | US2006174372. |
| Crv1 | US2007061919. |
| Crv1 | US2007061919. |
| Crv | US2007061919. CN1401772. U.S. Pat. No. 6,063,605. |
| Crv | US2007061919. AU784649B. U.S. Pat. No. 5,723,758. |
| | U.S. Pat. No. 5,616,319, U.S. Pat. No. 5,356,623, |
| | U.S. Pat. No. 5,322,687 |
| Crv1 | US57237 |
| Crv2 | CN1942582. WO9840490. |
| | US2007061919. UA75570. |
| | MXPA03006130. US2003167517. |
| | U.S. Pat. No. 6,107,278. U.S. Pat. No. 6,096,708. |
| | U.S. Pat. No. 5,073,632. |
| | U.S. Pat. No. 7,208,474. U.S. Pat. No. 7,244,880. |
| Crv3 | US2002152496. RU2278161. |
| | US2003054391. |
| Crv3 | U.S. Pat. No. 5,837,237. U.S. Pat. No. 5,723,756. |
| | U.S. Pat. No. 5,683,691. |
| | U.S. Pat. No. 5,104,974. U.S. Pat. No. 4,996,155. |
| Crv3 | U.S. Pat. No. 5,837,237. U.S. Pat. No. 5,723,756. |
| Crv5 | WO9840491. US2004018982. U.S. Pat. No. 6,166,195. |
| | US2001010932. U.S. Pat. No. 5,985,831. |
| | U.S. Pat. No. 5,824,792. US52815 |
| Crv5 | WO2007062064. US2001010932. |
| | U.S. Pat. No. 5,824,792. |
| Crv6 | WO2007062064, US2004018982, |
| | U.S. Pat. No. 5,973,231. U.S. Pat. No. 5,874,288. |
| | U.S. Pat. No. 5,236,843. US68310 |
| Crv6 | US2004018982. U.S. Pat. No. 6,166,195. |
| Crv7 | U.S. Pat. No. 6,048,839. U.S. Pat. No. 5,683,691. |
| | U.S. Pat. No. 5,378,625. US51870 |
| Crv7 | CN19521 |
| Crv8 | |
| Crv8 | |
| Crv8 | US2003017 |
| Crv8 | WO2006053473. US2007245430. |
| Crv8 | WO2006053 |
| Crv9 | US2007061919. |
| Crv9 | WO2005066 |
| Crv9 | US2007061919. U.S. Pat. No. 6,448,226. |
| | US2005097635. WO2005066202. |
| | U.S. Pat. No. 6,143,550. U.S. Pat. No. 6,028,246. |
| | U.S. Pat. No. 6,727,409. |
| Crv9 | US2005097635. WO2005066202. |
| Crv9 | U.S. Pat. No. 6,570,005. |
| Crv9 | AU784649B. US2007074308. |
| | US73618 |
| Crv11 | MXPA02008 |
| Crv12 | US2004018982. U.S. Pat. No. 6,166,195. |
| | U.S. Pat. No. 6,077,937. U.S. Pat. No. 5,824,792. |
| | U.S. Pat. No. 5,753,492. |
| Crv13 | US2004018982. U.S. Pat. No. 6,166,195. |
| | U.S. Pat. No. 6,077,937. U.S. Pat. No. 5,824,792. |
| | U.S. Pat. No. 5,753,492. |
| Crv14 | JP2007006895. U.S. Pat. No. 5,831,011. |
| Crv21 | U.S. Pat. No. 5,831,011. U.S. Pat. No. 5,670,365. |
| Crv22 | US2006218666. US2001010932. |
| | MXPA01004361. U.S. Pat. No. 5,824,792. |
| Crv22 | US2003229919. |
| Crv23 | US2006051822. US2003144192. |
| | UA75317. U.S. Pat. No. 6,399,330. U.S. Pat. No. 6,326,351. |
| | U.S. Pat. No. 6,949,626. |
| Crv26 | US2003150 |
| Crv28 | US2003150 |
| Crv31 | CA2410153. |
| Crv34 | US2003167 |
| Crv35 | US2003167522. |
| Crv37 | US2006051822. US2003144192. |
| | UA75317. U.S. Pat. No. 6,399,330. U.S. Pat. No. 6,326,351. |
| | U.S. Pat. No. 6,949,626. |
| Crv43 | US2005271 |
| Cvt1 | WO2007027776. |
| Cvt1 | U.S. Pat. No. 6,150,165. |
| Cvt2 | US2007163000. EP1681351. |
| | U.S. Pat. No. 6,686,452. U.S. Pat. No. 6,537,756. |

TABLE 3
Hybrid Insecticidal Crystal Proteins and Patents.

| Patent No. | Holotype Toxin |
|---|---|
| US2008020967 | Cry29Aa |
| US2008040827 | Cry1Ca |
| US2007245430 | Cry8Aa |
| US2008016596 | Cry8Aa |
| US2008020968 | Cry1Cb |

TABLE 4
Patents Relating to Other Hybrid Insecticidal Crystal Proteins.

| Holotype Toxin | Patent No. |
|---|---|
| Cry23A, Cry37A | U.S. Pat. No. 7,214,788 |
| Cry1A | U.S. Pat. No. 7,019,197 |
| Cry1A, Cry1B | U.S. Pat. No. 6,320,100 |
| Cry1A, Cry1C | AU2001285900B |
| Cry23A, Cry37A | US2007208168 |
| Cry3A, Cry1I, Cry1B | WO0134811 |
| Cry3A, Cry3B, Cry3C | US2004033523 |
| Cry1A, Cry1C, Cry1E, | U.S. Pat. No. 6,780,408 |
| Cry1G | |
| Cry1A, Cry1F | US2008047034 |

Novel formulations comprising AVP can be used to control, kill and/or inhibit pests such as insects. In some embodiments, the method of controlling an insect comprises: applying Bt (Bacillus thuringiensis) protein to an insect; and applying an AVP to said insect. The foregoing application can be applied concomitantly and/or sequentially, and either in the same or separate compositions. In some embodiments, the Bt protein and the AVP may be applied to (Bt protein)-resistant insects. The ratio of Bt to AVP, on a dry weight basis, can be selected from at least about the following ratios: 99:1, 95:5, 90:10, 85:15, 80:20, 75:25, 70:30, 65:35, 60:40, 55:45, 50:50, 45:55, 40:60, 35:65, 30:70, 25:75, 20:80, 15:85, 10:90, 5:95 and 1:99, or any combination of any two of these values. The total concentration of Bt and AVP in the composition is selected from the following percent concentrations: 0, 1, 5, 10, 15, 20, 25, 30, 35, 40, 45, 50, 55, 60, 65, 70, 75, 80, 85, 90, 95, 99 or 100%, or any range between any two of these values, and the remaining percentage of the composition is comprised of excipients.

In come embodiments, AVP can be included in a formulation, for example, a formulation composed of a polar aprotic solvent, and or water, and or where the polar aprotic solvent is present in an amount of 1-99 wt %, the polar protic solvent is present in an amount of 1-99 wt %, and the water is present in an amount of 0-98 wt %. In some embodiments, the formulations include an AVP, and another insecticidal polypeptide, for example, a Bt protein. In some embodiments, the Bt protein is Dipel. The polar aprotic solvent formulations are especially effective when they contain MSO. MSO is a methylated seed oil and surfactant blend that uses methyl esters of soya oil in amounts of between about 80 and 85 percent petroleum oil with 15 to 20 percent surfactant.

In some embodiments, the composition comprises both a Bt (Bacillus thuringiensis) protein and an AVP. The composition can be in the ratio of Bt to AVP, on a dry weight basis, from about any or all of the following ratios: 99:1, 95:5, 90:10, 85:15, 80:20, 75:25, 70:30, 65:35, 60:40, 55:45, 50:50, 45:55, 40:60, 35:65, 30:70, 25:75, 20:80, 15:85, 10:90, 5:95 and 1:99, or any combination of any two of these values. In some embodiments, the composition can have a ratio of Bt to AVP, on a on a dry weight basis, selected from about the following ratios: 0:50, 45:55, 40:60, 35:65, 30:70, 25:75, 20:80, 15:85, 10:90, 5:95, 1:99, 0.5:99.5, 0.1:99.9 and 0.01:99.99 or any combination of any two of these values.

In some embodiments, a composition comprising AVP and one or more additional pesticides, for example, AaITI (sodium channel gating modifier from the scorpion, Androctonus australis hector), and/or Bt (a toxin derived from Bacillus thuringiensis, for example, Bti derived from Bacillus thuringiensis serotype israelensis), can be formulated.

In some embodiments, AVP can be combined with permethrin. Permethrin is an insecticide that is commercially available (e.g., Nix®) and known to those having ordinary skill in the art. In some embodiments, the method of controlling an insect comprises: applying permethrin to an insect; and applying an AVP to said insect. The foregoing application can be applied concomitantly and/or sequentially, and either in the same or separate compositions. In some embodiments, permethrin and the AVP may be applied to (permethrin)-resistant insects. The ratio of permethrin to AVP, on a dry weight basis, can be selected from at least about the following ratios: 99:1, 95:5, 90:10, 85:15, 80:20, 75:25, 70:30, 65:35, 60:40, 55:45, 50:50, 45:55, 40:60, 35:65, 30:70, 25:75, 20:80, 15:85, 10:90, 5:95 and 1:99, or any combination of any two of these values. The total concentration of permethrin and AVP in the composition is selected from the following percent concentrations: 0, 1, 5, 10, 15, 20, 25, 30, 35, 40, 45, 50, 55, 60, 65, 70, 75, 80, 85, 90, 95, 99 or 100%, or any range between any two of these values, and the remaining percentage of the composition is comprised of excipients.

In some embodiments, the composition comprises both a permethrin and an AVP. The composition can be in the ratio of permethrin to AVP, on a dry weight basis, from about any or all of the following ratios: 99:1, 95:5, 90:10, 85:15, 80:20, 75:25, 70:30, 65:35, 60:40, 55:45, 50:50, 45:55, 40:60, 35:65, 30:70, 25:75, 20:80, 15:85, 10:90, 5:95 and 1:99, or any combination of any two of these values. In some embodiments, the composition can have a ratio of permethrin to AVP, on a on a dry weight basis, selected from about the following ratios: 0:50, 45:55, 40:60, 35:65, 30:70, 25:75, 20:80, 15:85, 10:90, 5:95, 1:99, 0.5:99.5, 0.1:99.9 and 0.01:99.99 or any combination of any two of these values.

In some embodiments, an AVP can be combined with AaIT1. The protein toxin, AalT1, is a sodium channel site 4 toxin from North African desert scorpion (Androctonus australis). AVP targets the insect sodium channel receptor site 3, which inhibits the inactivation of the channel. AaIT1 is a site 4 toxin, which forces the insect sodium channel to open by lowering the activation reaction energy barrier. Thus, site 3 and site 4 toxins both are involved in causing the insect sodium channel to open, albeit in different ways.

In some embodiments, the method of controlling an insect comprises: applying AaIT1 to an insect; and applying an AVP to said insect. The foregoing application can be applied concomitantly and/or sequentially, and either in the same or separate compositions. In some embodiments, AaIT1 and the AVP may be applied to (AaIT1)-resistant insects. The ratio of AaIT1 to AVP, on a dry weight basis, can be selected from at least about the following ratios: 99:1, 95:5, 90:10, 85:15, 80:20, 75:25, 70:30, 65:35, 60:40, 55:45, 50:50, 45:55, 40:60, 35:65, 30:70, 25:75, 20:80, 15:85, 10:90, 5:95 and 1:99, or any combination of any two of these values. The total concentration of AaIT1 and AVP in the composition is selected from the following percent concentrations: 0, 1, 5, 10, 15, 20, 25, 30, 35, 40, 45, 50, 55, 60, 65, 70, 75, 80, 85, 90, 95, 99 or 100%, or any range between any two of these values, and the remaining percentage of the composition is comprised of excipients.

In some embodiments, the composition comprises both an AaIT1 and an AVP. The composition can be in the ratio of AaITI to AVP, on a dry weight basis, from about any or all of the following ratios: 99:1, 95:5, 90:10, 85:15, 80:20, 75:25, 70:30, 65:35, 60:40, 55:45, 50:50, 45:55, 40:60, 35:65, 30:70, 25:75, 20:80, 15:85, 10:90, 5:95 and 1:99, or any combination of any two of these values. In some embodiments, the composition can have a ratio of AaITI to AVP, on a on a dry weight basis, selected from about the following ratios: 0:50, 45:55, 40:60, 35:65, 30:70, 25:75, 20:80, 15:85, 10:90, 5:95, 1:99, 0.5:99.5, 0.1:99.9 and 0.01:99.99 or any combination of any two of these values.

### Crops and Pests

Specific crop pests and insects that may be controlled by these methods include the following: Dictyoptera (cockroaches); Isoptera (termites); Orthoptera (locusts, grasshoppers and crickets); Diptera (house flies, mosquito, tsetse fly, crane-flies and fruit flies); Hymenoptera (ants, wasps, bees, saw-flies, ichneumon flies and gall-wasps); Anoplura (biting and sucking lice); Siphonaptera (fleas); and Hemiptera (bugs and aphids), as well as arachnids such as Acari (ticks and mites), and the parasites that each of these organisms harbor.

“Pest” includes, but is not limited to: insects, fungi, bacteria, nematodes, mites, ticks, and the like.

Insect pests include, but are not limited to, insects selected from the orders Coleoptera, Diptera, Hymenoptera, Lepidoptera, Mallophaga, Homoptera, Hemiptera, Orthroptera, Thysanoptera, Dermaptera, Isoptera, Anoplura, Siphonaptera, Trichoptera, and the like. More particularly, insect pests include Coleoptera, Lepidoptera, and Diptera.

Insects of suitable agricultural, household and/or medical/veterinary importance for treatment with the insecticidal polypeptides include, but are not limited to, members of the following classes and orders:
The order Coleoptera includes the suborders Adephaga and Polyphaga. Suborder Adephaga includes the superfamilies Caraboidea and Gyrinoidea. Suborder Polyphaga includes the superfamilies Hydrophiloidea, Staphylinoidea, Cantharoidea, Cleroidea, Elateroidea, Dascilloidea, Dryopoidea, Byrrhoidea, Cucujoidea, Meloidea, Mordelloidea, Tenebrionoidea, Bostrichoidea, Scarabaeoidea, Cerambycoidea, Chrysomeloidea, and Curculionoidea. Superfamily Caraboidea includes the families Cicindelidae, Carabidae, and Dytiscidae. Superfamily Gyrinoidea includes the family Gyrinidae. Superfamily Hydrophiloidea includes the family Hydrophilidae. Superfamily Staphylinoidea includes the families Silphidae and Staphylinidae. Superfamily Cantharoidea includes the families Cantharidae and Lampyridae. Superfamily Cleroidea includes the families Cleridae and Dermestidae. Superfamily Elateroidea includes the families Elateridae and Buprestidae. Superfamily Cucujoidea includes the family Coccinellidae. Superfamily Meloidea includes the family Meloidae. Superfamily Tenebrionoidea includes the family Tenebrionidae. Superfamily Scarabaeoidea includes the families Passalidae and Scarabaeidae. Superfamily Cerambycoidea includes the family Cerambycidae. Superfamily Chrysomeloidea includes the family Chrysomelidae. Superfamily Curculionoidea includes the families Curculionidae and Scolytidae.

Examples of Coleoptera include, but are not limited to: the American bean weevil Acanthoscelides obtectus, the leaf beetle Agelastica alni, click beetles (Agriotes lineatus, Agriotes obscurus, Agriotes bicolor), the grain beetle Ahasverus advena, the summer schafer Amphimallon solstitialis, the furniture beetle Anobium punctatum, Anthonomus spp. (weevils), the Pygmy mangold beetle Atomaria linearis, carpet beetles (Anthrenus spp., Attagenus spp.), the cowpea weevil Callosobruchus maculates, the fried fruit beetle Carpophilus hemipterus, the cabbage seedpod weevil Ceutorhynchus assimilis, the rape winter stem weevil Ceutorhynchus picitarsis, the wireworms Conoderus vespertinus and Conoderus falli, the banana weevil Cosmopolites sordidus, the New Zealand grass grub Costelytra zealandica, the June beetle Cotinis nitida, the sunflower stem weevil Cylindrocopturus adspersus, the larder beetle Dermestes lardarius, the corn rootworms Diabrotica virgifera, Diabrotica virgifera virgifera, and Diabrotica barberi, the Mexican bean beetle Epilachna varivestis, the old house borer Hylotropes bajulus, the lucerne weevil Hypera postica, the shiny spider beetle Gibbium psylloides, the cigarette beetle Lasioderma serricorne, the Colorado potato beetle Leptinotarsa decemlineata, Lyctus beetles (Lyctus spp.), the pollen beetle Meligethes aeneus, the common cockshafer Melolontha melolontha, the American spider beetle Mezium americanum, the golden spider beetle Niptus hololeucus, the grain beetles Oryzaephilus surinamensis and Oryzaephilus mercator, the black vine weevil Otiorhynchus sulcatus, the mustard beetle Phaedon cochleariae, the crucifer flea beetle Phyllotreta cruciferae, the striped flea beetle Phyllotreta striolata, the cabbage steam flea beetle Psylliodes chrysocephala, Ptinus spp. (spider beetles), the lesser grain borer Rhizopertha dominica, the pea and been weevil Sitona lineatus, the rice and granary beetles Sitophilus oryzae and Sitophilus granaries, the red sunflower seed weevil Smicronyx fulvus, the drugstore beetle Stegobium paniceum, the yellow mealworm beetle Tenebrio molitor, the flour beetles Tribolium castaneum and Tribolium confusum, warehouse and cabinet beetles (Trogoderma spp.), and the sunflower beetle Zygogramma exclamation's.

Examples of Dermaptera (earwigs) include, but are not limited to: the European earwig Forficula auricularia, and the striped earwig Labidura riparia.

Examples of Dictvontera include, but are not limited to: the oriental cockroach Blatta orientalis, the German cockroach Blatella germanica, the Madeira cockroach Leucophaea maderae, the American cockroach Periplaneta americana, and the smokybrown cockroach Periplaneta fuliginosa.

Examples of Diplonoda include, but are not limited to: the spotted snake millipede Blaniulus guttulatus, the flat-back millipede Brachydesmus superus, and the greenhouse millipede Oxidus gracilis.

The order Diptera includes the Suborders Nematocera, Brachycera, and Cyclorrhapha. Suborder Nematocera includes the families Tipulidae, Psychodidae, Culicidae, Ceratopogonidae, Chironomidae, Simuliidae, Bibionidae, and Cecidomyiidae. Suborder Brachycera includes the families Stratiomyidae, Tabanidae, Therevidae, Asilidae, Mydidae, Bombyliidae, and Dolichopodidae. Suborder Cyclorrhapha includes the Divisions Aschiza and Aschiza. Division Aschiza includes the families Phoridae, Syrphidae, and Conopidae. Division Aschiza includes the Sections Acalyptratae and Calyptratae. Section Acalyptratae includes the families Otitidae, Tephritidae, Agromyzidae, and Drosophilidae. Section Calyptratae includes the families Hippoboscidae, Oestridae, Tachinidae, Anthomyiidae, Muscidae, Calliphoridae, and Sarcophagidae.

Examples of Diptera include, but are not limited to: the house fly (Musca domestica), the African tumbu fly (Cordylobia anthropophaga), biting midges (Culicoides spp.), bee louse (Braula spp.), the beet fly Pegomyia betae, blackflies (Cnephia spp., Eusimulium spp., Simulium spp.), bot flies (Cuterebra spp., Gastrophilus spp., Oestrus spp.), craneflies (Tipula spp.), eye gnats (Hippelates spp.), filth-breeding flies (Calliphora spp., Fannia spp., Hermetia spp., Lucilia spp., Musca spp., Muscina spp., Phaenicia spp., Phormia spp.), flesh flies (Sarcophaga spp., Wohlfahrtia spp.); the flit fly Oscinellafrit, fruitflies (Dacus spp., Drosophila spp.), head and canon flies (Hydrotea spp.), the hessian fly Mayetiola destructor, horn and buffalo flies (Haematobia spp.), horse and deer flies (Chrysops spp., Haematopota spp., Tabanus spp.), louse flies (Lipoptena spp., Lynchia spp., and Pseudolynchia spp.), medflies (Ceratitus spp.), mosquitoes (Aedes spp., Anopheles spp., Culex spp., Psorophora spp.), sandflies (Phlebotomus spp., Lutzomyia-spp.), screw-worm flies (Chtysomya bezziana and Cochliomyia hominivorax), sheep keds (Melophagus spp.); stable flies (Stomoxys spp.), tsetse flies (Glossina spp.), and warble flies (Hypoderma spp.).

Examples of Isontera (termites) include, but are not limited to: species from the familes Hodotennitidae, Kalotermitidae, Mastotermitidae, Rhinotennitidae, Serritermitidae, Termitidae, Termopsidae;

Examples of Heteroptera include, but are not limited to: the bed bug Cimex lectularius, the cotton stainer Dysdercus intermedius, the Sunn pest Eurygaster integriceps, the tarnished plant bug Lygus lineolaris, the green stink bug Nezara antennata, the southern green stink bug Nezara viridula, and the triatomid bugs Panstrogylus megistus, Rhodnius ecuadoriensis, Rhodnius pallescans, Rhodnius prolixus, Rhodnius robustus, Triatoma dimidiata, Triatoma infestans, and Triatoma sordida.

Examples of Homoptera include, but are not limited to: the California red scale Aonidiella aurantii, the black bean aphid Aphisffabae, the cotton or melon aphid Aphis gossypii, the green apple aphid Aphis pomi, the citrus spiny whitefly Aleurocanthus spiniferus, the oleander scale Aspidiotus hederae, the sweet potato whitefly Bemesia tabaci, the cabbage aphid Brevicoryne brassicae, the pear psylla Cacopsylla pyricola, the currant aphid Cryptomyzus ribis, the grape phylloxera Daktulosphaira vitifoliae, the citrus psylla Diaphorina citri, the potato leafhopper Empoascaffabae, the bean leafhopper Empoasca solana, the vine leafhopper Empoasca vitis, the woolly aphid Eriosoma lanigerum, the European fruit scale Eulecanium corni, the mealy plum aphid Hyalopterus arundinis, the small brown planthopper Laodelphax striatellus, the potato aphid Macrosiphum euphorbiae, the green peach aphid Myzuspersicae, the green rice leafhopper Nephotettix cinticeps, the brown planthopper Nilaparvata lugens, gall-forming aphids (Pemphigus spp.), the hop aphid Phorodon humuli, the bird-cherry aphid Rhopalosiphum padi, the black scale Saissetia oleae, the greenbug Schizaphis graminum, the grain aphid Sitobion avenae, and the greenhouse whitefly Trialeurodes vaporariorum.

Examples of Isopoda include, but are not limited to: the common pillbug Armadillidium vulgare and the common woodlouse Oniscus asellus.

The order Lepidoptera includes the families Papilionidae, Pieridae, Lycaenidae, Nymphalidae, Danaidae, Satyridae, Hesperiidae, Sphingidae, Saturniidae, Geometridae, Arctiidae, Noctuidae, Lymantriidae, Sesiidae, and Tineidae.

Examples of Lepidoptera include, but are not limited to: Adoxophyes orana (summer fruit tortrix moth), Agrotis ipsolon (black cutworm), Archips podana (fruit tree tortrix moth), Bucculatrix pyrivorella (pear leafminer), Bucculatrix thurberiella (cotton leaf perforator), Bupalus piniarius (pine looper), Carpocapsa pomonella (codling moth), Chilo suppressalis (striped rice borer), Choristoneura fumiferana (eastern spruce budworm), Cochylis hospes (banded sunflower moth), Diatraea grandiosella (southwestern corn borer), Earls insulana (Egyptian bollworm), Euphestia kuehniella (Mediterranean flour moth), Eupoecilia ambiguella (European grape berry moth), Euproctis chrysorrhoea (brown-tail moth), Euproctis subflava (oriental tussock moth), Galleria mellonella (greater wax moth), Helicoverpa armigera (cotton bollworm), Helicoverpa zea (cotton bollworm), Heliothis virescens (tobacco budworm), Hofmannophila pseudopretella (brown house moth), Homeosoma electellum (sunflower moth), Homona magnanima (oriental tea tree tortrix moth), Lithocolletis blancardella (spotted tentiform leafminer), Lymantria dispar (gypsy moth), Malacosoma neustria (tent caterpillar), Mamestra brassicae (cabbage armyworm), Mamestra configurata (Bertha armyworm), the hornworms Manduca sexta and Manuduca quinquemaculata, Operophtera brumata (winter moth), Ostrinia nubilalis (European corn borer), Panolis flammea (pine beauty moth), Pectinophora gossypiella (pink bollworm), Phyllocnistis citrella (citrus leafminer), Pieris brassicae (cabbage white butterfly), Plutella xylostella (diamondback moth), Rachiplusia ni (soybean looper), Spilosoma virginica (yellow bear moth), Spodoptera exigua (beet armyworm), Spodoptera frugiperda (fall armyworm), Spodoptera littoralis (cotton leafworin), Spodoptera litura (common cutworm), Spodoptera praefica (yellowstriped armyworm), Sylepta derogata (cotton leaf roller), Tineola bisselliella (webbing clothes moth), Tineola pellionella (case-making clothes moth), Tortrix viridana (European oak leafroller), Trichoplusia ni (cabbage looper), and Yponomeuta padella (small ermine moth).

Examples of Orthoptera include, but are not limited to: the common cricket Acheta domesticus, tree locusts (Anacridium spp.), the migratory locust Locusta migratoria, the twostriped grasshopper Melanoplus bivittatus, the differential grasshopper Melanoplus dfferentialis, the redlegged grasshopper Melanoplus femurrubrum, the migratory grasshopper Melanoplus sanguinipes, the northern mole cricket Neocurtilla hexadectyla, the red locust Nomadacris septemfasciata, the shortwinged mole cricket Scapteriscus abbreviatus, the southern mole cricket Scapteriscus borellii, the tawny mole cricket Scapteriscus vicinus, and the desert locust Schistocerca gregaria.

Examples of Phthiraptera include, but are not limited to: the cattle biting louse Bovicola bovis, biting lice (Damalinia spp.), the cat louse Felicola subrostrata, the shortnosed cattle louse Haematopinus eloysternus, the tail-switch louse Haematopinus quadriperiussus, the hog louse Haematopinus suis, the face louse Linognathus ovillus, the foot louse Linognathus pedalis, the dog sucking louse Linognathus setosus, the long-nosed cattle louse Linognathus vituli, the chicken body louse Menacanthus stramineus, the poultry shaft louse Menopon gallinae, the human body louse Pediculus humanus, the pubic louse Phthirus pubis, the little blue cattle louse Solenopotes capillatus, and the dog biting louse Trichodectes canis.

Examples of Psocoptera include, but are not limited to: the booklice Liposcelis bostrychophila, Liposcelis decolor, Liposcelis entomophila, and Trogium pulsatorium.

Examples of Siphonaptera include, but are not limited to: the bird flea Ceratophyllus gallinae, the dog flea Ctenocephalides canis, the catflea Ctenocephalides fells, the humanflea Pulex irritans, and the oriental rat flea Xenopsylla cheopis.

Examples of Symphyla include, but are not limited to: the garden symphylan Scutigerella immaculate.

Examples of Thysanura include, but are not limited to: the gray silverfish Ctenolepisma longicaudata, the four-lined silverfish Ctenolepisma quadriseriata, the common silverfish Lepisma saccharina, and the firebrat Thennobia domestica;

Examples of Thysanoptera include, but are not limited to: the tobacco thrips Frankliniella fusca, the flower thrips Frankliniella intonsa, the western flower thrips Frankliniella occidentalis, the cotton bud thrips Frankliniella schultzei, the banded greenhouse thrips Hercinothrips femoralis, the soybean thrips Neohydatothrips variabilis, Kelly's citrus thrips Pezothrips kellyanus, the avocado thrips Scirtothrips perseae, the melon thrips Thrips palmi, and the onion thrips Thrips tabaci.

Examples of Nematodes include, but are not limited to: parasitic nematodes such as root-knot, cyst, and lesion nematodes, including Heterodera spp., Meloidogyne spp., and Globodera spp.; particularly members of the cyst nematodes, including, but not limited to: Heterodera glycines (soybean cyst nematode); Heterodera schachtii (beet cyst nematode); Heterodera avenae (cereal cyst nematode); and Globodera rostochiensis and Globodera pailida (potato cyst nematodes). Lesion nematodes include, but are not limited to: Pratylenchus spp.

Other insect species susceptible to an AVP of the present disclosure includes: arthropod pests which cause public and animal health concerns, for example, mosquitos for example, mosquitoes from the genera Aedes, Anopheles and Culex, from ticks, flea, and flies etc.

In one embodiment, the insecticidal compositions comprising the polypeptides, polynucleotides, cells, vectors, etc., can be employed to treat ectoparasites. Ectoparasites include, but are not limited to: fleas, ticks, mange, mites, mosquitoes, nuisance and biting flies, lice, and combinations comprising one or more of the foregoing ectoparasites. The term “fleas” includes the usual or accidental species of parasitic flea of the order Siphonaptera, and in particular the species Ctenocephalides, in particular C. fells and C. cams, rat fleas (Xenopsylla cheopis) and human fleas (Pulex irritans).

Insect pests of the invention for the major crops include, but are not limited to: Maize: Ostrinia nubilalis, European corn borer; Agrotis ipsilon, black cutworm; Helicoverpa zea, corn earworm; Spodoptera fugiperda, fall armyworm; Diatraea grandiosella, southwestern corn borer; Elasmopalpus lignosellus, lesser cornstalk borer; Diatraea saccharalis, surgarcane borer; Diabrotica virgifera, western corn rootworm; Diabrotica longicornis barberi, northern corn rootworm; Diabrotica undecimpunctata howardi, southern corn rootworm; Melanotus spp., wireworms; Cyclocephala borealis, northern masked chafer (white grub); Cyclocephala immaculata, southern masked chafer (white grub); Popillia japonica, Japanese beetle; Chaetocnema pulicaria, corn flea beetle; Sphenophorus maidis, maize billbug; Rhopalosiphum maidis, corn leaf aphid; Anuraphis maidiradicis, corn root aphid; Blissus leucopterus leucopterus, chinch bug; Melanoplus femurrubrum, redlegged grasshopper; Melanoplus sanguinipes, migratory grasshopper; Hylemya platura, seedcorn maggot; Agromyza parvicornis, corn blot leafminer; Anaphothrips obscrurus, grass thrips; Solenopsis milesta, thief ant; Tetranychus urticae, twospotted spider mite; Sorghum: Chilo partellus, sorghum borer; Spodoptera fugiperda, fall armyworm; Helicoverpa zea, corn earworm; Elasmopalpus lignosellus, lesser cornstalk borer; Feltia subterranea, granulate cutworm; Phyllophaga crinita, white grub; Eleodes, Conoderus, and Aeolus spp., wireworms; Oulema melanopus, cereal leaf beetle; Chaetocnema pulicaria, corn flea beetle; Sphenophorus maidis, maize billbug; Rhopalosiphum maidis, corn leaf aphid; Sipha flava, yellow sugarcane aphid; Blissus leucopterus leucopterus, chinch bug; Contarinia sorghicola, sorghum midge; Tetranychus cinnabarinus, carmine spider mite; Tetranychus urticae, twospotted spider mite; Wheat: Pseudaletia unipunctata, army worm; Spodoptera frugiperda, fall armyworm; Elasmopalpus lignosellus, lesser cornstalk borer; Agrotis orthogonia, western cutworm; Elasmopalpus lignosellus, lesser cornstalk borer; Oulema melanopus, cereal leaf beetle; Hypera punctata, clover leaf weevil; Diabrotica undecimpunctata howardi, southern corn rootworm; Russian wheat aphid; Schizaphis graminum, greenbug; Macrosiphum avenae, English grain aphid; Melanoplus femurrubrum, redlegged grasshopper; Melanoplus differentialis, differential grasshopper; Melanoplus sanguinipes, migratory grasshopper; Mayetiola destructor, Hessian fly; Sitodiplosis mosellana, wheat midge; Meromyza americana, wheat stem maggot; Hylemya coarctata, wheat bulb fly; Frankliniella fusca, tobacco thrips; Cephus cinctus, wheat stem sawfly; Aceria tulipae, wheat curl mite; Sunflower: Suleima helianthana, sunflower bud moth; Homoeosoma electellum, sunflower moth; Zygogramma exclamationis, sunflower beetle; Bothyrus gibbosus, carrot beetle; Neolasioptera murtfeldtiana, sunflower seed midge; Cotton: Heliothis virescens, cotton budworm; Helicoverpa zea, cotton bollworm; Spodoptera exigua, beet armyworm; Pectinophora gossypiella, pink bollworm; Anthonomus grandis, boll weevil; Aphis gossypii, cotton aphid; Pseudatomoscelis seriatus, cotton fleahopper; Trialeurodes abutilonea, banded winged whitefly; Lygus lineolaris, tarnished plant bug; Melanoplus femurrubrum, redlegged grasshopper; Melanoplus differentialis, differential grasshopper; Thrips tabaci, onion thrips; Frankliniella fusca, tobacco thrips; Tetranychus cinnabarinus, carmine spider mite; Tetranychus urticae, twospotted spider mite; Rice: Diatraea saccharalis, sugarcane borer; Spodoptera fugiperda, fall armyworm; Helicoverpa zea, corn earworm; Colaspis brunnea, grape colaspis; Lissorhoptrus oryzophilus, rice water weevil; Sitophilus oryzae, rice weevil; Nephotettix nigropictus, rice leafhopper; Blissus leucopterus, chinch bug; Acrosternum hilare, green stink bug; Soybean: Pseudoplusia includens, soybean looper; Anticarsia gemmatalis, velvet bean caterpillar; Plathypena scabra, green clover worm; Ostrinia nubilalis, European corn borer; Agrotis ipsilon, black cutworm; Spodoptera exigua, beet armyworm; Heliothis virescens, cotton budworm; Helicoverpa zea, cotton bollworm; Epilachna varivestis, Mexican bean beetle; Myzus persicae, green peach aphid; Empoasca fabae, potato leafhopper; Acrosternum hilare, green stink bug; Melanoplus femurrubrum, redlegged grasshopper; Melanoplus differentialis, differential grasshopper; Hylemya platura, seedcorn maggot; Sericothrips variabilis, soybean thrips; Thrips tabaci, onion thrips; Tetranychus turkestani, strawberry spider mite; Tetranychus urticae, twospotted spider mite; Barley: Ostrinia nubilalis, European corn borer; Agrotis ipsilon, black cutworm; Schizaphis graminum, greenbug; Blissus leucopterus leucopterus, chinch bug; Acrosternum hilare, green stink bug; Euschistus servus, brown stink bug; Delia platura, seedcorn maggot; Mayetiola destructor, Hessian fly; Petrobia latens, brown wheat mite; Oil Seed Rape: Brevicoryne brassicae, cabbage aphid; Phyllotreta cruciferae, Flea beetle; Mamestra configurata, Bertha armyworm; Plutella xylostella, Diamond-back moth; Delia ssp., Root maggots.

In some embodiments, the insecticidal compositions can be employed to treat combinations comprising one or more of the foregoing insects.

The insects that are susceptible to the peptides of this invention include but are not limited to the following: Cyt toxins affect familes such as: Blattaria, Coleoptera, Collembola, Diptera, Echinostomida, Hemiptera, Hymenoptera, Isoptera, Lepidoptera, Neuroptera, Orthoptera, Rhabditida, Siphonoptera, Thysanoptera. Genus-Species are indicated as follows: Actebia-fennica, Agrotis-ipsilon, A.-segetum, Anticarsia-gemmatalis, Argyrotaenia-citrana, Artogeia-rapae, Bombyx-mori, Busseola-fusca, Cacyreus-marshall, Chilo-suppressalis, Christoneura-fumiferana, C.-occidentalis, C.-pinus pinus, C.-rosacena, Cnaphalocrocis-medinalis, Conopomorpha-cramerella, Ctenopsuestis-obliquana, Cydia-pomonella, Danaus-plexippus, Diatraea-saccharallis, D.-grandiosella, Earias-vittella, Elasmolpalpus-lignoselius, Eldana-saccharina, Ephestia-kuehniella, Epinotia-aporema, Epiphyas-postvittana, Galleria-mellonella, Genus-Species, Helicoverpa-zea, H.-punctigera, H-armigera, Heliothis-virescens, Hyphantria-cunea, Lambdina-fiscellaria, Leguminivora-glycinivorella, Lobesia-botrana, Lymantria-dispar, Malacosoma-disstria, Mamestra-brassicae, M. configurata, Manduca-sexta, Marasmia-patnalis, Maruca-vitrata, Orgyia-leucostigma, Ostrinia-nubilalis, O.-fhrnacalis, Pandemis-pyrusana, Pectinophora-gossypiella, Perileucoptera-coffeella, Phthorimaea-opercullela, Pianotortrix-octo, Piatynota-stultana, Pieris-brassicae, Plodia-interpunctala, Plutella-xylostella, Pseudoplusia-includens, Rachiplusia-nu, Sciropophaga-incertulas, Sesamia-calamistis, Spilosoma-virginica, Spodoptera-exigua, S.-frugiperda, S.-littoralis, S.-exempta, S.-litura, Tecia-solanivora, Thaumetopoea-pityocampa, Trichoplusia-ni, Wiseana-cervinata, Wiseana-copularis, Wiseana-jocosa, Blattaria-Blattella, Collembola-Xenylla, C.-Folsomia, Echinostomida-Fasciola, Hemiptera-Oncopeltrus, He.-Bemisia, He.-Macrosiphum, He.-Rhopalosiphum, He.-Myzus, Hymenoptera-Diprion, Hy.-Apis, Hy.-Macrocentrus, Hy.-Meteorus, Hy.-Nasonia, Hy.-Solenopsis, Isopoda-Porcellio, Isoptera-Reticulitermes, Orthoptera-Achta, Prostigmata-Tetranychus, Rhabitida-Acrobeloides, R.-Caenorhabditis, R.-Distolabrellus, R.-Panagrellus, R.-Pristionchus, R.-Pratylenchus, R.-Ancylostoma, R.-Nippostrongylus, R.-Panagrellus, R.-Haemonchus, R.-Meloidogyne, and Siphonaptera-Ctenocephalides.

The present disclosure provides methods for plant transformation, which may be used for transformation of any plant species, including, but not limited to, monocots and dicots. Crops for which a transgenic approach or plaint incorporated protectants (PIP) would be an especially useful approach include, but are not limited to: alfalfa, cotton, tomato, maize, wheat, corn, sweet corn, lucerne, soybean, sorghum, field pea, linseed, safflower, rapeseed, oil seed rape, rice, soybean, barley, sunflower, trees (including coniferous and deciduous), flowers (including those grown commercially and in greenhouses), field lupins, switchgrass, sugarcane, potatoes, tomatoes, tobacco, crucifers, peppers, sugarbeet, barley, and oilseed rape, Brassica sp., rye, millet, peanuts, sweet potato, cassava, coffee, coconut, pineapple, citrus trees, cocoa, tea, banana, avocado, fig, guava, mango, olive, papaya, cashew, macadamia, almond, oats, vegetables, ornamentals, and conifers.

### EXAMPLES

The Examples in this specification are not intended to, and should not be used to, limit the invention; they are provided only to illustrate the invention.

### Example 1

Generation of Av3+2 Expression in K. lactis Yeast Strains

Generation of an Av3+2 Expression K. lactis yeast strain was accomplished by first generating an Av3+2 peptide sequence: GSRSCCPCYWGGCPWGQNCYPEGCSGPKV (SEQ ID NO: 18). An Av3+2 peptide expression vector was generated based on the pKLAC1 yeast expression vector form New England Biolab: Av3+2 peptide was expressed as a secretion peptide with acetamidase gene expression as the selection marker. The expression vector of pLB102 was linearized by the digestion with the restriction enzyme SacII; the linear pLB102 plasmid was then transformed into K. lactis cell by electroporation; 96 of resulting positive transformation colonies were cultured. Seed culture of the production strain for inoculation of the 2 L fermentation was preceded for 24 hours in the seed medium containing 3% solulys 095K+3% glucose and 50 μg/mL Kanamycin. Then 30 mL seed culture was used to inoculate 2 L fermentation tank with 1 L batch medium containing 1 L basal salt media (BMS (g/L): Solulys 095K 40, suppressor 3519±0.1 mL, 85% phosphoric acid 13 mL, CaSO₄·5, K₂SO₄ 9.1, MgSO₄·7H₂O 7.5, KOH 2.1, (NH₄)₂SO₄ 5, Dextrose 10) with 1.2% Pichia Trace metals (PTM (g/L): CuSO₄·5H₂O 6, NaI 0.08, MgSO₄·H₂O 3, NaMoO₄·2H₂O 0.2, H₃BO₃ 0.02, CoCl₂·6H₂O 0.5, ZnCl₂ 20, FeSO₄·6H₂O 65, H₂SO₄ 5 mL) and 2 mL 5% suppressor 7153. Batch phase of fermentation continued for 6 hours with controlled temperature at 27 C°, pH 4.80 and dissolved oxygen at 15%. After 6 hour batch fermentation, temperature was dropped to 23.5 C° and feeding of sugar alcohol started and continued for 120 hours with temperature control at 23.5 C° for the rest of fermentation process. Feed media was fed at a gradually increased rates: 3.4 mL/hr for 24 hours, 4.4 mL/hr for 30 hours, 7.2 mL/hr for 24 hours, 8.8 mL/hr for 12 hours and 11 mL/hr until feed medium was totally consumed. Their yield was evaluated in a 48-well deep-well plate culture via HPLC (evaluation of the spent medium because the Av3+2 peptide was designed for secretion). Of the colonies that were analyzed, the strain designated “pLB102-YCT-18,” was found to have the highest Av3+2 peptide yield, and was subsequently selected for further studies. The pLB102-YCT-18 strain was then used in standard fermentation conditions, for example, the yeast fermentation conditions explained in the foregoing text, to produce Av3+2 peptide. In the unoptimized fermentation conditions, the pLB102-YCT-18 strain produced an Av3+2 peptide yield of 116 mg/L of peptide in the supernatant.

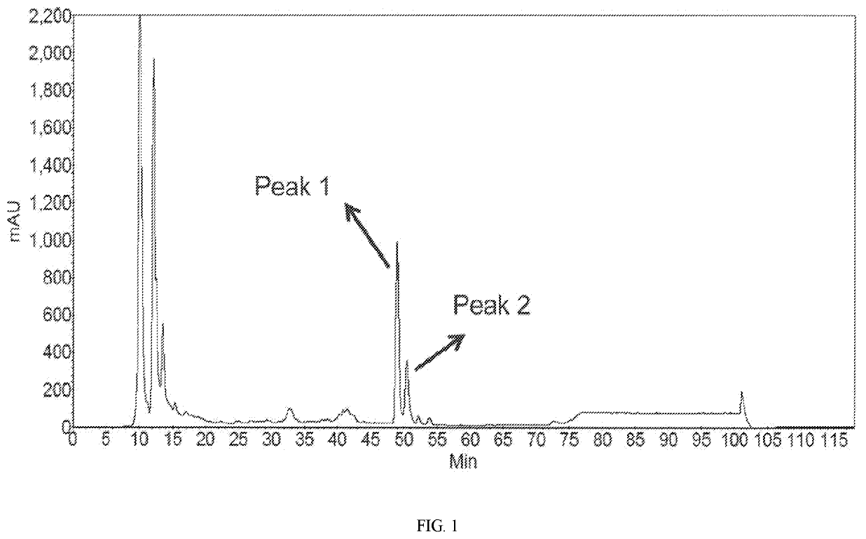

Reverse-phase HPLC was used to purify Av3+2 peptide from the fermentation beer (i.e., spent medium) via monolithic C18 columns using water with 0.1% Trifloroacetic acid, and acetonitrile as the mobile phase. FIG. 1. An elution protocol using 20-40% acetonitrile was used for Av3+2 peptide purification, in which Av3+2 peptide was eluted between a range of 34-36% acetonitrile. At the Av3+2 peptide retention time, there were two separated peaks from the fermentation beer: “Peak 1” and “Peak 2,” reflecting two different isoforms of the Av3+2 peptide. FIG. 1.

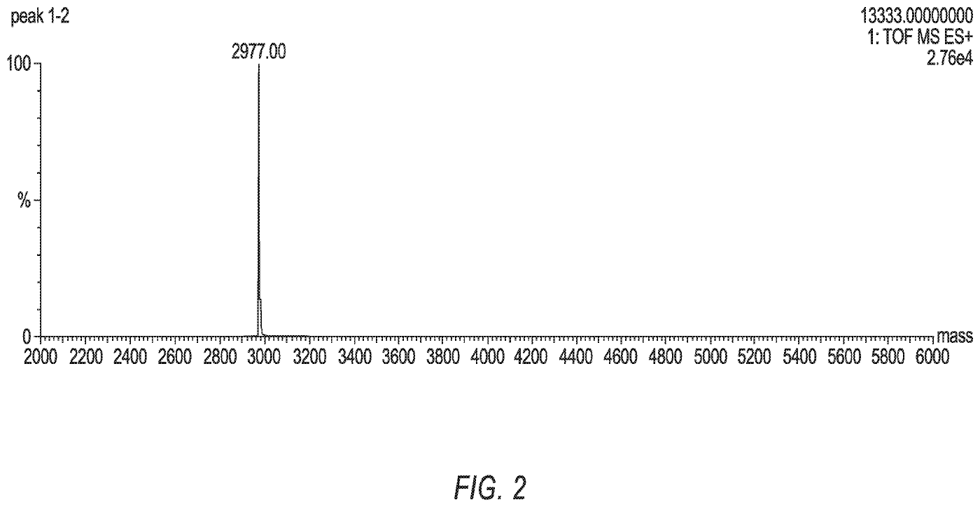

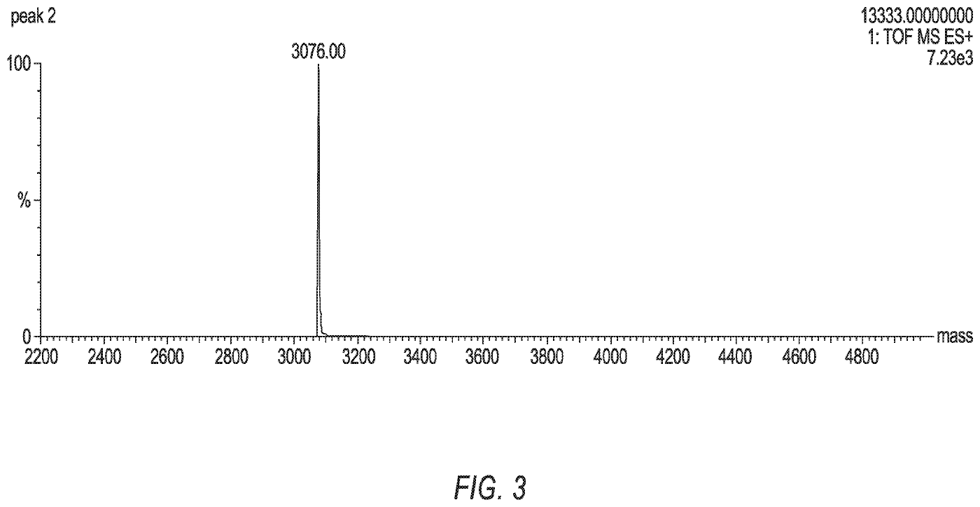

The peaks observed in FIG. 1 (i.e., “Peak 1” and “Peak 2”) of the Av3+2 peptide were purified by rpHPLC and subjected to LC/MS identification using a Waters/Micromass ESI-TOF mass spectrometer on-line with an Agilent HPLC system. The LC/MS indicated that Peak 1 reflected a cellularly modified Av3+2 peptide with C-terminal valine cleaved (Av3+2−C1) having an amino acid sequence: GSRSCCPCYWGGCPWGQNCYPEGCSGPK (SEQ ID NO:19). FIG. 2. The LC/MS indicated that the isoform resulting in Peak 2 is the Av3+2 peptide with sequence: GSRSCCPCYWGGCPWGQNCYPEGCSGPKV (SEQ ID NO: 18) (i.e., the initial sequence used to generate the Av3+2 expressing K. lactis yeast strain; see above). FIG. 3.

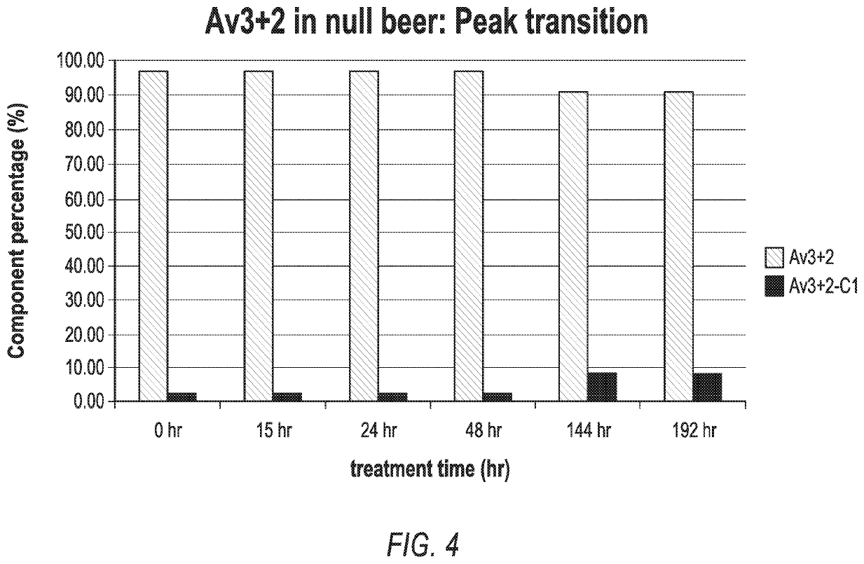

The pLB102-YCT-18 strain's apparent expression of two Av3+2 peptides, i.e., Av3+2 and Av3+2−C1, raised the question as to whether the C-Val cleavage observed in Peak 1 (SEQ ID NO: 19) occurred during peptide expression and secretion, or whether it occurred in the spent medium/fermentation beer as a result of endogenous proteases secreted from K. lactis. To answer this question, Av3+2 peptide was purified according to the abovementioned methods, and incubated with the fermentation beer in concert with untransformed K. lactis strain (i.e., a null strain) for 8 days at room temperature (RT). FIG. 4. As shown in FIG. 4, incubating purified Av3+2 peptide in fermentation beer alongside untransformed K. lactis did not result in any apparent C-Val cleavage; this indicated that C-Val cleavage most likely occurs during the peptide expression and/or secretion process. Furthermore, FIG. 4 also demonstrates the stability of Av3+2 peptide at RT.

### Example 2

Generation of a K. lactis Strain Expressing Wild Type (WT) Av3 Peptide

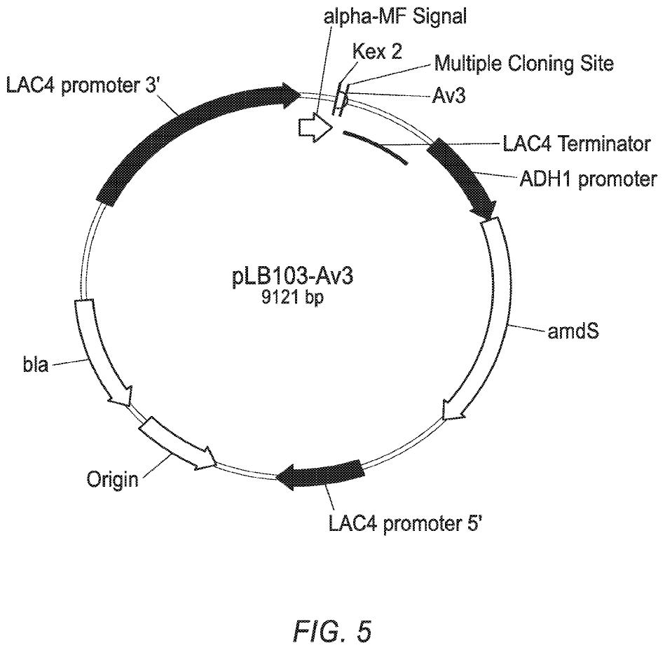

A K. lactis strain expressing the wild type Av3 peptide sequence: RSCCPCYWGGCPWGQNCYPEGCSGPKV (SEQ ID NO:1) was generated using an expression vector based off of the pKLAC1 yeast expression vector (available from New England Biolabs; see above); wherein the Av3 peptide was expressed as secretion peptide, using the acetamidase gene (amdS) as a selection marker (i.e., using amdS to permit transformed K. lactis cells to grow in medium containing acetamide). FIG. 5.

Briefly, a pLB103 expression vector was linearized via digestion with a restriction enzyme (SacII); the linear pLB103 plasmid was then transformed into K. lactis cell by electroporation; 96 of resulting positive transformation colonies were cultured according to the methods described above and in the foregoing example, and the resulting peptide yield was evaluated using a 48-well, deep-well plate culture via HPLC evaluation of the spent medium (i.e., because Av3 peptide was designed for secretion). Based on this evaluation, the strain designated as “pLB103-YCT-1”, was determined to produce the highest Av3 peptide yield, and was therefore selected for further studies. Accordingly, to produce WT Av3 peptide, the pLB103-YCT-1 strain was grown under the fermentation conditions described above. When using unoptimized fermentation conditions, the pLB103-YCT-1 strain of transformed K. lactis produced a WT Av3 peptide yield of 173 mg/L of peptide to supernatant.

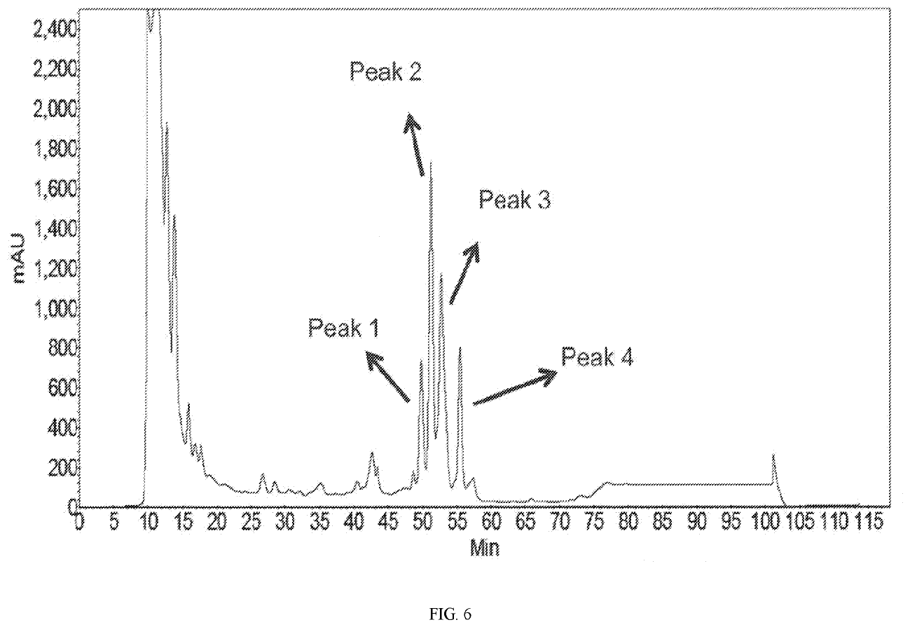

Similar to the previous example, reverse-phase HPLC was used to purify the WT Av3 peptide from the fermentation beer (or spent medium) via monolithic C18 columns using water with 0.1% Trifloroacetic acid and acetonitrile as the mobile phase. An elution protocol using 20-40% acetonitrile was used for the WT Av3 peptide purification. The WT Av3 peptide was eluted between 34-37% of acetonitrile. Analysis of the WT Av3 fermentation beer by rpHPLC chromatograph revealed that there were four separate peaks eluted out at the Av3 peptide retention time, correlating to different isoforms of the Av3 peptide. FIG. 6.

Pursuant to the versatility of the WT Av3 expression vector design, the N-terminal arginine of the WT Av3 peptide can be recognized and cleaved by the Kex2 protease, and its C-terminal valine can be cleaved by a heretofore unknown protease. Therefore, the expressed Av3 peptide may produce four different isoforms: (1) WT Av3, or “native” Av3; (2) WT Av3 with its N-Arg cleaved (Av3-N1); (3) WT Av3 with its C-Val cleaved (Av3-C1); and (4) WT Av3 with both N-Arg and C-Val cleaved (Av3-NC).

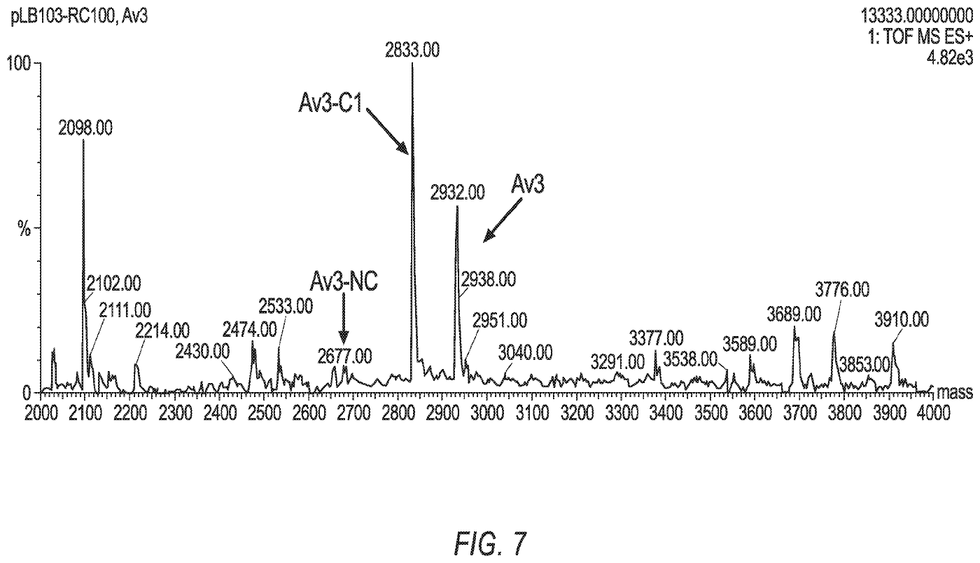

LC/MS using a Waters/Micromass ESI-TOF mass spectrometer on-line with an Agilent HPLC system was performed to identify the Av3 isoforms contained the fermentation beer, resulting in the detection of three isoforms: (1) Av3; (2) Av3-C1; and (3) Av3-NC, FIG. 7. Peak 2 was identified as Av3-C1 having the amino acid sequence: RSCCPCYWGGCPWGQNCYPEGCSGPK (SEQ ID NO:3). FIG. 7. Peak 3 was identified as WT Av3, having the amino acid sequence: RSCCPCYWGGCPWGQNCYPEGCSGPKV (SEQ ID NO: 1). FIG. 7.

### Example 3

### Generation of Av3 Variant Peptides Resistant to Endogenous Cleavage

Briefly, the Av3 peptide incurs two endogenous cleavages during its expression: an N-terminal Arginine (N-Arg) cleavage event, and a C-terminal Valine (C-Val) cleavage event. The N-Arg cleavage event is likely due to the introduction of a Kex2 protease cleavage site within the expression ORF design. To prevent this cleavage, N-Arg can be mutated into any other amino acid; however, any mutations and/or modifications to the polypeptide must avoid a loss of the peptide's original activity. One such modification, the addition of a “Gly-Ser” di-peptide (GS), can be introduced to the N-terminal to prevent the N-Arg cleavage as indicated from Av3+2 expression (see Example 2), however, the addition of GS results in an unacceptable loss in activity. The C-Val cleavage event appears to be caused by some unknown endogenous protease from the yeast, K. lactis. Adjacent to the C-Val is a lysine residue, which is the recognition site of many proteases; thus, a mutation of this lysine was theorized as possible method to prevent the observed C-terminal cleavage. C-Val itself can be mutated to prevent this cleavage, or alternatively, even the removal of C-Val is an option if it does not result in a loss of activity. Moreover, one or more of the foregoing strategies may be combined generate an AVP that is (1) expressed as single product from the expression strain; and (2) exhibits minimal activity loss. Accordingly, the following mutation strategies were developed, with the following results:

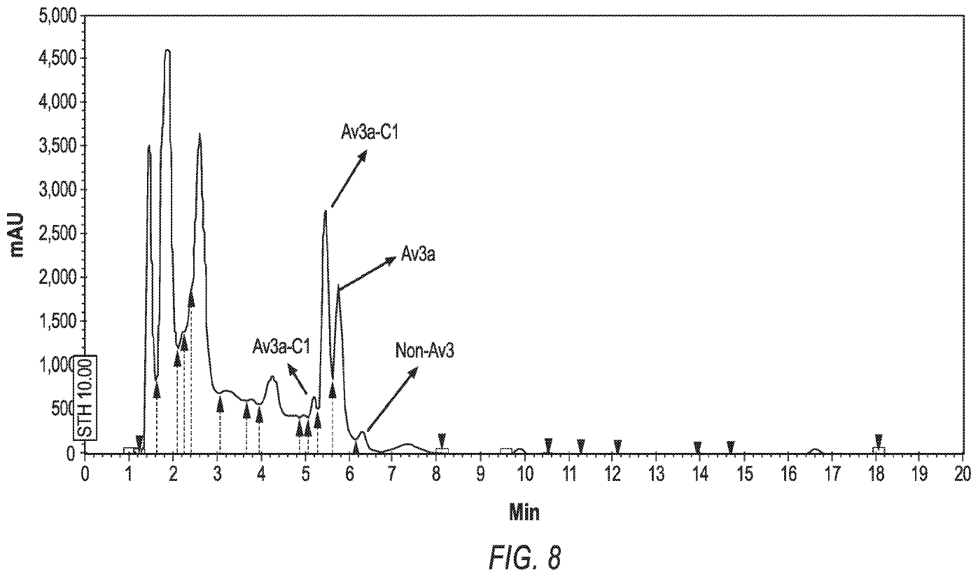

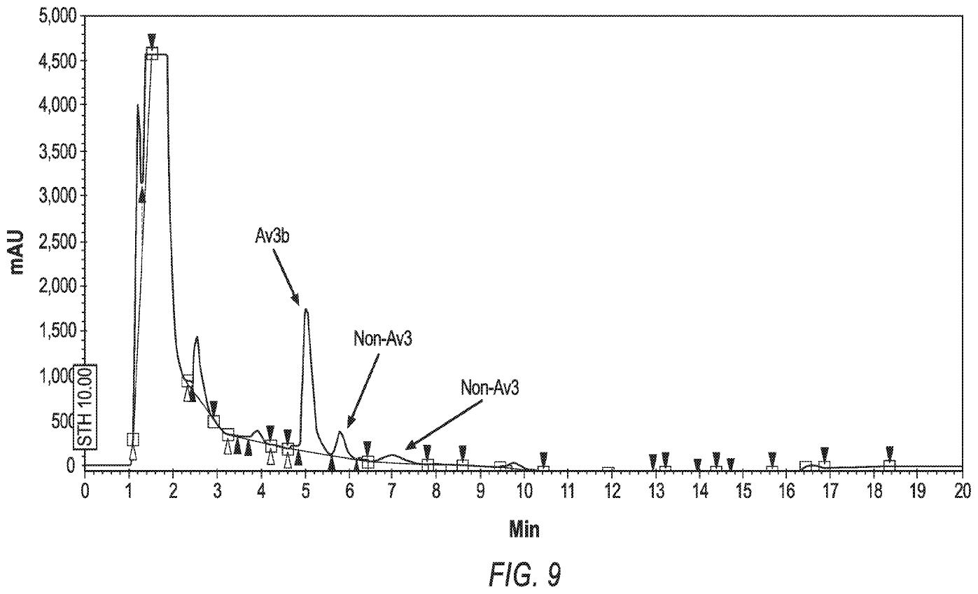

pLB102a (Av3 + 2a, K28A):
(SEQ ID NO: 20)
GSRSCCPCYWGGCPWGQNCYPEGCSGPAV
(did not prevent C-Val cleavage).
pLB102b (AV3 + 2b, P27A-K28A):
(SEQ ID NO: 21)
GSRSCCPCYWGGCPWGQNCYPEGCSGAAV
(did not prevent C-Val cleavage).
pLB102c (Av3 + 2c, K28H):
(SEQ ID NO: 22)
GSRSCCPCYWGGCPWGQNCYPEGCSGPHV
(did not prevent C-Val cleavage).
pLB102d (Av3 + 2d, P27A-K28H):
(SEQ ID NO: 23)
GSRSCCPCYWGGCPWGQNCYPEGCSGAHV
(did not prevent C-Val cleavage).
pLB102e (Av3 + 2e, K28D):
(SEQ ID NO: 24)
GSRSCCPCYWGGCPWGQNCYPEGCSGPDV
(did not prevent C-Val cleavage).
pLB102f (Av3 + 2f, AV):
(SEQ ID NO: 25)
GSRSCCPCYWGGCPWGQNCYPEGCSGPK
(produced a single peptide).
pLB102g (Av3 + 2g, V29T):
(SEQ ID NO: 26)
GSRSCCPCYWGGCPWGQNCYPEGCSGPKT
(did not prevent C-Val cleavage).
pLB102h (Av3 + 2h, V29A):
(SEQ ID NO: 27)
GSRSCCPCYWGGCPWGQNCYPEGCSGPKA
(did not prevent C-Val cleavage).
V29P:
(SEQ ID NO: 28)
GSRSCCPCYWGGCPWGQNCYPEGCSGPKP
(did not prevent C-Val cleavage).
K28Q:
(SEQ ID NO: 29)
GSRSCCPCYWGGCPWGQNCYPEGCSGPQV
(did not prevent C-Val cleavage).
pLB103a (Av3a, RIK):
(SEQ ID NO: 2)
KSCCPCYWGGCPWGQNCYPEGCSGPKV.
FIG. 8.
pLB103b (Av3b, RIK-AV):
(SEQ ID NO: 4)
KSCCPCYWGGCPWGQNCYPEGCSGPK.
FIG. 9.

The pLB103a-YCT strain expressing an Av3a peptide with a mutation of R1K relative to the wild-type sequence produced two peptides: Av3a and Av3a-C1, with and without C-Val cleavage respectively. The N-terminal cleavage was not observed in both initial screen and in later experiments. FIG. 8.

The pLB103b-YCT-3 strain resulted in the expression of an Av3b peptide with a mutation of R1K and AC-Val relative to the wild-type sequence. This strain resulted in the production of only one Av3 variant peptide isoform, as confirmed by LC/MS. FIG. 9.

In summary, mutations resulting in the removal of C-terminal valine from Av3 peptide can effectively prevent further C-terminal cleavage during expression of the peptide from K. lactis. And, a mutation of N-terminal Arginine to Lysine from Av3 peptide can effectively prevent N-terminal cleavage during expression of the peptide from K. lactis.

### Example 4

### Comparing the Insecticidal Activities of AVPs

A housefly injection assay was performed, which revealed that AC-Val reduced the Av3+2 peptide activities. Av3+2 and Av3+2−C1 peptide were injected into houseflies, and knock-down activities (or paralysis activities) of the both peptides were observed as early as 1-hour post-injection (with knock-down effects likely to occur earlier). Recovery of the knock-down flies was observed even at 1000 pmol/g dose (overnight observation). No recovery was observed at 5000 and 20000 pmol/g doses.

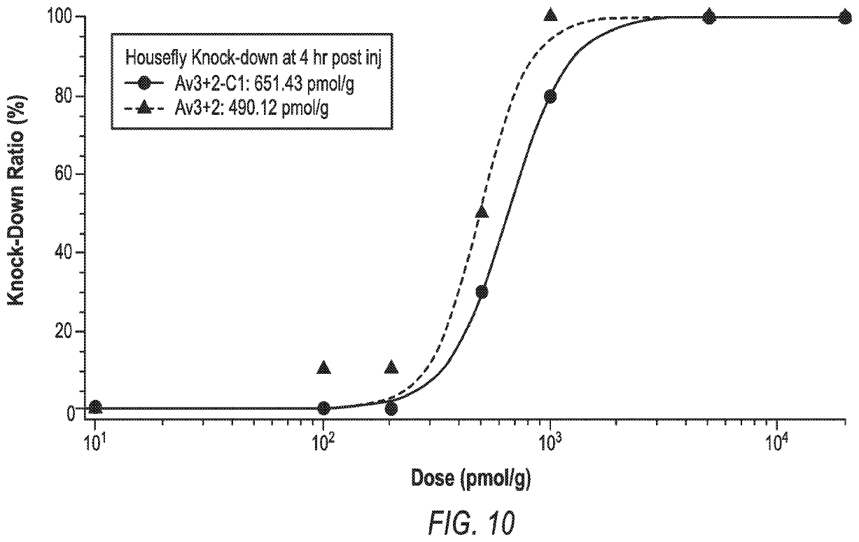

The lowest dose to produce knock-down activity within the fSSIt 4 hours post injection was 100 pmol/g for Av3+2, and 500 pmol/g for Av3+2−C1. FIG. 10.

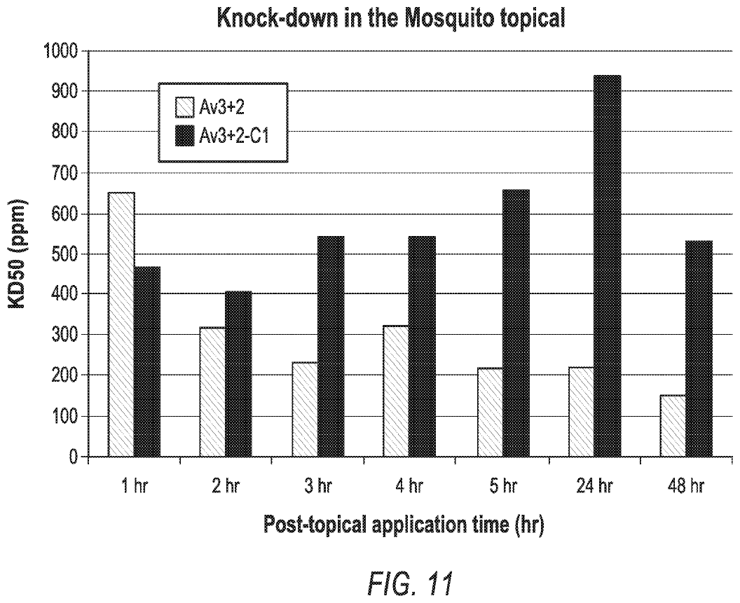

In a mosquito bioassay, the mutation of AC-Val reduced Av3+2 peptide activities. Av3+2 and Av3+2−C1 peptide were shown to fast paralyze and eventually kill adult mosquitos upon topical application. FIG. 11. Both the Av3+2 and Av3+-C1 peptides caused knock-down in Aedes aegypti as early as 1-hour post-topical application. The Av3+2 knock-down effect reached its maximum efficacy at around 3-hours, with limited recovery thereafter. At 3-hours, the KD₅₀ was 235.71 ppm. For the Av3+2−C1 peptide, knock-down effect reached a maximum at around 2-hours, however, recovery was observed; and at 3-hours the KD₅₀ was 543.12 ppm. FIG. 11.

At almost all the recorded times, Av3+2 had lower KD₅₀ than Av3+2−C1. FIG. 11. At 3-hours, the KD₅₀ of Av3+2−C1 was almost 2-fold higher than that of Av3+2, indicating that the truncation of C-Val reduced the paralyzing effect of the Av3+2 peptide.

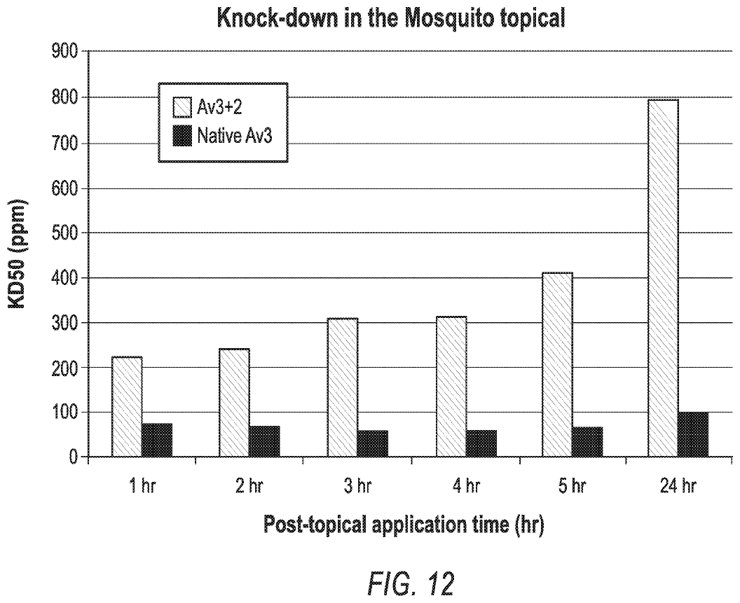

The addition of “GS” dramatically reduced the Native Av3 peptide activities. FIG. 12. Native Av3 and Av3+2 were evaluated using the adult mosquito topical bioassay (see above). Both Av3 and Av3+2 resulted in the knock-down of Aedes aegypti mosquitoes as early as 1-hour post-topical application. Native Av3 knock-down effect reached its zenith after 1-hour, with no obvious recovery observed thereafter. At 3-hours, the KD₅₀ was 58 ppm. FIG. 12. The Av3+2 knock-down effect reached its maximum after 1 to 2 hours—with recovery observed over time. At 3-hours, the KD₅₀ was 312.57 ppm, roughly 5-fold less than that observed for native Av3. At all record times, up to 24 hours, native Av3 had a 3-fold to 8-fold lower KD₅₀ than Av3+2, indicating that the addition of “GS” at the N-terminus dramatically reduces the insecticidal activity of native Av3.

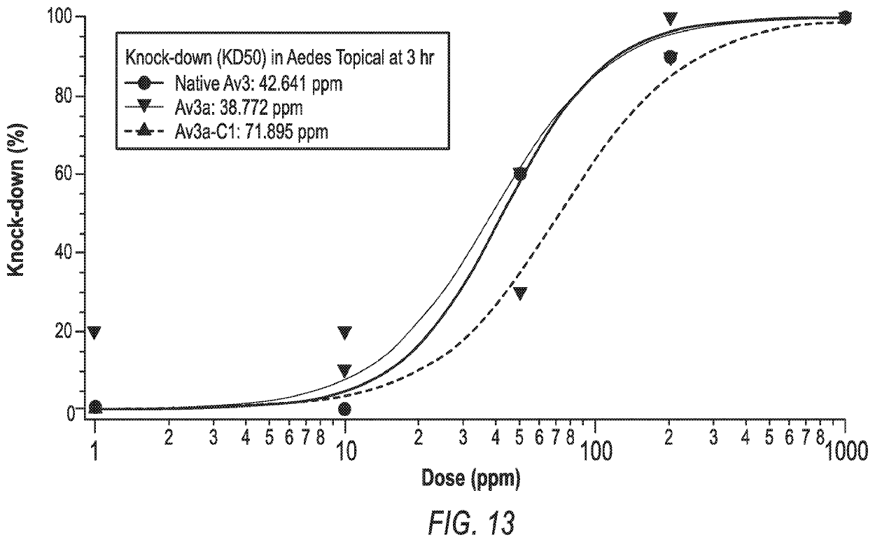

Native Av3, R1K mutant Av3 (Av3a) and R1K+AC-Val double mutant Av3 (Av3a-C1, or Av3b) were evaluated in the adult mosquito topical bioassay. The adult mosquitoes were ordered from Benzon Research. The adult mosquitoes were immobilized using CO₂ gas and transferred onto a CO₂ pad with CO₂ flowing to keep them immobilized during topical applications. A hand microapplicator equipped with 1 cc glass syringe with a 30 gouge straight needle was used for the application of droplet. 0.25 μl droplet of toxin solution per mosquito was pushed out from the needle and gently applied to the ventral side of the mosquito abdomen. The treated mosquitoes were put into a 2.5 oz. clear cup with moisture filter paper and lid with breathing holes. Score the treated mosquitoes for paralysis or death within 24 hours post-application. Native Av3 had a KD₅₀ of 42.6 ppm at 3-hours post application. Av3a had KD₅₀ of 38.8 ppm at 3-hours post application, which was the same as native Av3, indicating that R1K mutation did not cause a reduction in activity. Finally, Av3a-C1 had a KD₅₀ of 71.9 ppm at 3-hours post application, indicating it was two-fold less active than the native Av3, but much better than Av3+2 (200-300 ppm) and Av3+2−C1 (500-900 ppm). FIG. 13.

In conclusion, addition of a “GS” dipeptide to the N-terminal of the Av3 peptide was deleterious to the bio-activities of the Av3 peptide, and therefore is not acceptable. The R1K mutation on the Av3 peptide protected the N-terminus from endogenous cleavage during yeast expression, and had negligible effect on the peptide bio-activities. The AC-Val mutation prevented the C-terminal cleavage of the Av3 peptide and had minor but acceptable reduction it its effect on the peptide bio-activities. Overall, based on their ability to protect against endogenous protease-cleaving during expression, and their concomitant minimal effect on activity, the R1K and AC-Val double mutations were chosen as the mutation/modification strategy for the wild-type Av3 peptide, i.e. Av3b (or Av3a-C1) peptide, for insecticidal peptide development.

### Example 5

Improvement of Av3b Yield from a K. lactis Yeast Strain

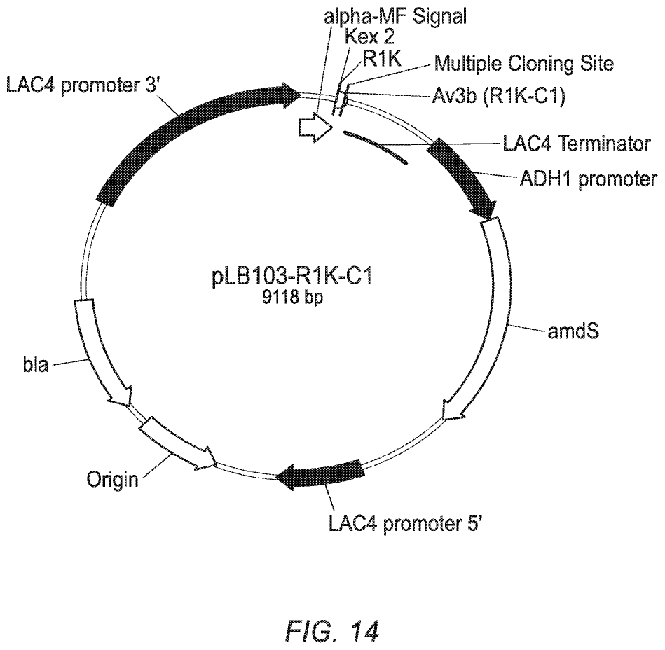

A single expression cassette containing the Av3b vector, and expressed from the pLB103b-YCT-3 strain, was generated from a single Av3b expression cassette K. lactis vector (pLB103b), due to its high-yield in the primary screen. Accordingly, this strain was selected as the Av3b production strain for this peptide. FIG. 14. Fermentation with the pLB103b-YCT-3 strain was performed using the conditions described in Example 1. Under these conditions, the pLB103b-YCT-3 strain had a yield of about 100 mg/L of peptide per fermentation broth.

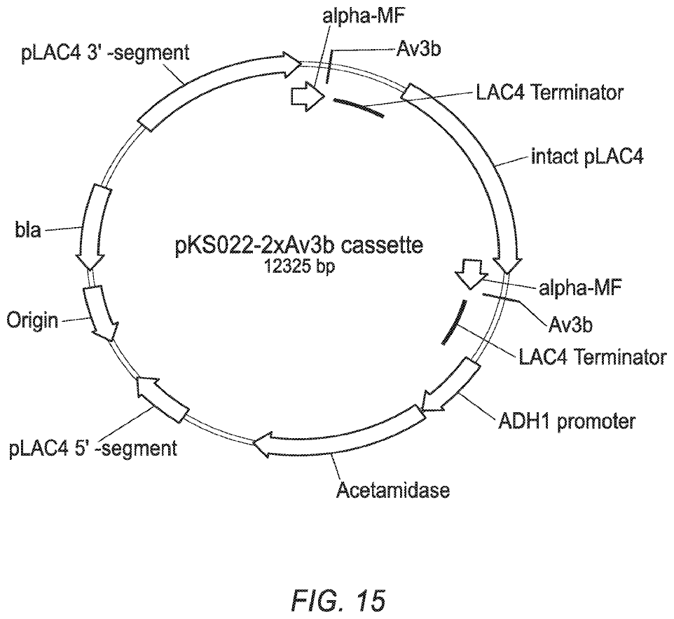

Next, a double Av3b expression cassette K. lactis expression vector, designated as pKS022, was generated by inserting a second full Av3b expression cassette into the pLB103b vector. FIG. 15. Based on the primary screen and secondary screen, two strains were identified as showing the highest Av3b yield: pKS022-YCT-38-14, and pKS022-YCT-53-24. A culture of both these strains was performed using the current standard fermentation procedure optimized for hybrid-F2 strains (Batch #:17-153-96-034-1 & 2), and detailed herein. Both pKS022-YCT-38-14 and pKS022-YCT-53-24 strains yielded about 2 g/L peptide per supernatant (fermentation broth), representing a 20-fold increase in yield compared to the single expression cassette strain.

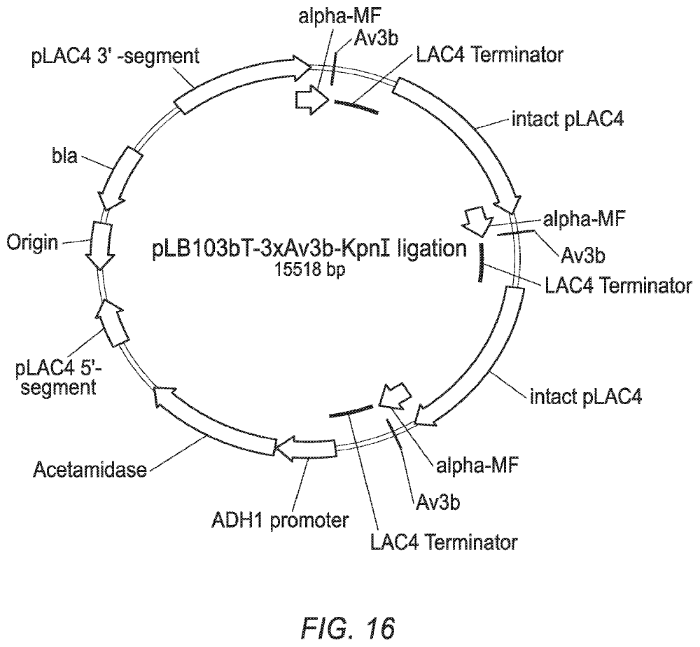

A triple Av3b expression cassette K. lactis expression vector, designated as pLB103bT, was the generated by inserting a third full Av3b expression cassette into pKS022 vector. FIG. 16, however, yield from the triple cassette expression strains showed similar or lower yield when compared to the pKS022 strain.

### Example 6

### Insecticidal Activity Against Houseflies Using Av3 Peptide and AVPs

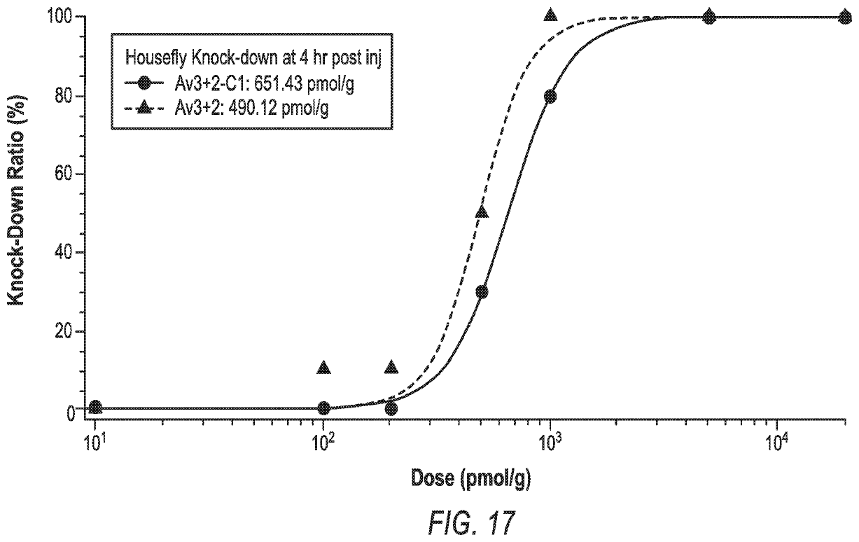

Av3+2 was found to be toxic to houseflies in an injection bioassay. Av3+2 and Av3+2−C1 peptide were injected into houseflies; the knock-down activities (or paralysis activities) of both peptides were observed as early as 1-hour post-injection. Recovery of knock-down flies was observed even at 1000 pmol/g dose (from overnight observation). No recovery was observed at 5000 and 20000 pmol/g doses. The lowest dose resulting in knock-down activity during the f first 4-hours post-injection was 100 pmol/g for Av3+2, and 500 pmol/g for Av3+2−C1. The KD₅₀ at 4-hours was 490 pmol/g for Av3+2 and 651 pmol/g for Av3+2−C1. FIG. 17.

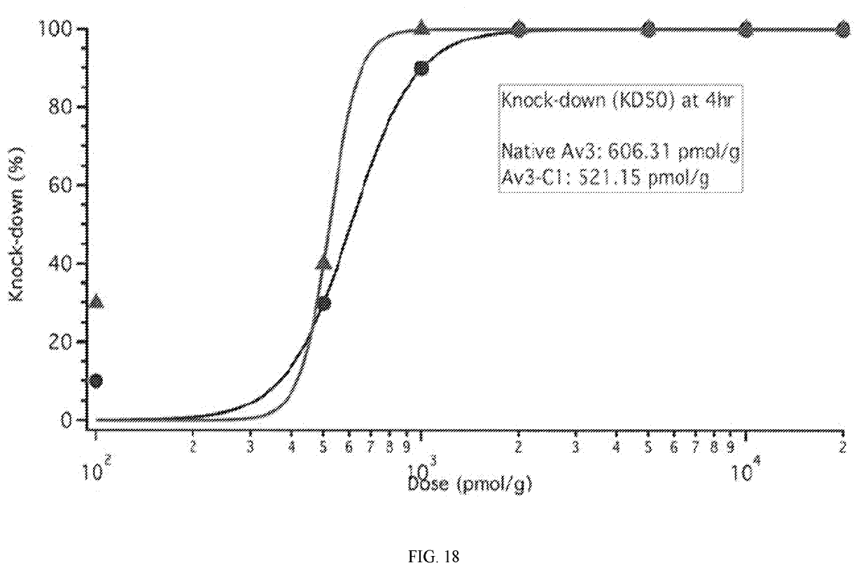

Native Av3 was found to be toxic to houseflies by after injection. Av3 and Av3-C1 peptide were injected into houseflies, and knock-down activities (or paralysis activities) of both peptides was observed as early as 1-hour post-injection. Recovery of the knock-down flies was very rare, even at a 100 pmol/g dose (based on overnight observation). The KD₅₀ at 4-hours was 606 pmol/g for Av3 and 521 pmol/g for Av3-C1. FIG. 18. Deletion of C-Val appeared not to have an effect on the activity of the peptide. Again, Av3 and Av3-C1 had no potent lethal activity to houseflies.

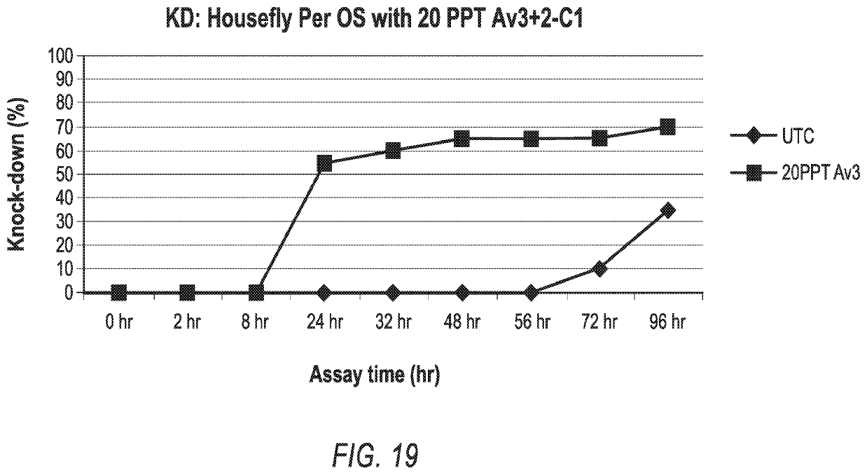

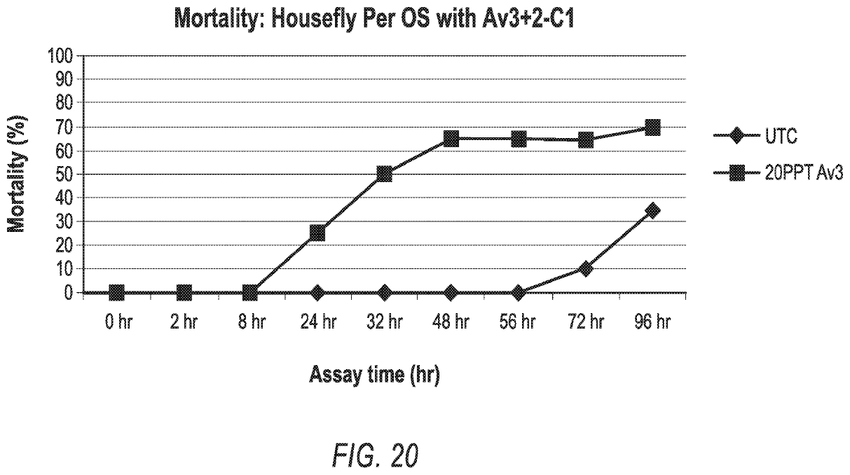

Additionally, Av3+2−C1 was found to be toxic to houseflies when consumed and/or upon oral contact. The oral toxicity of Av3+2−C1 peptide was evaluated in the housefly feeding assay by mixing 20 PPT Av3+2−C1 with fly food (1:1 sugar: dry milk) and observed for 96 hours. Knock-down and dead flies were observed starting at 24-hour post-feeding and accumulated with time. FIG. 19. Unlike the housefly injection bioassay, recovery from paralysis in the feeding assay was not observed, likely due to the fact that the houseflies were continuously dosed in the later bioassay. FIG. 20.

### Example 7

### Oral Insecticidal Activity Against Tobacco Hornworm Using Av3 Peptides and AVPs

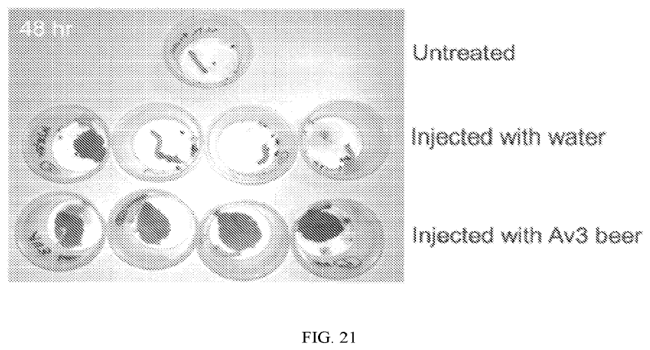

Av3 fermentation beer was found to be toxic to Manduca sexta. A 2 L Fermentation was performed with the native Av3 expression strain, and the pLB103-YCT-1 strain. The resulting fermentation beer was filtered to remove the yeast cells, and then concentrated using a TFC2 membrane. The concentrated Av3 beer was directly injected into 3rd instar tobacco hornworm (Manduca sexta). Injected Manduca in the untreated control group, and in the water injection control group, appeared normal after 72 hours post-injection. Leaf consumption started around 7 hours post injection, and at 72 hours post-injection all the tobacco leaves were consumed. FIG. 21. However, the Manduca worms injected with Av3 beer did not consume tobacco leaves until 64 hours post-injection; at 72 hours post-injection, only some leaves were consumed. FIG. 21. Observable uncontrollable body twitching of all the worms injected with Av3 beer occurred starting at 16 hours post-injection, and continued throughout the bioassay. One out of 4 worms in this group died, with another one was dying at the 72 hours post injection.

### Oral Insecticidal Activity Against Mosquito Larva Using Av3 Peptides and AVPs

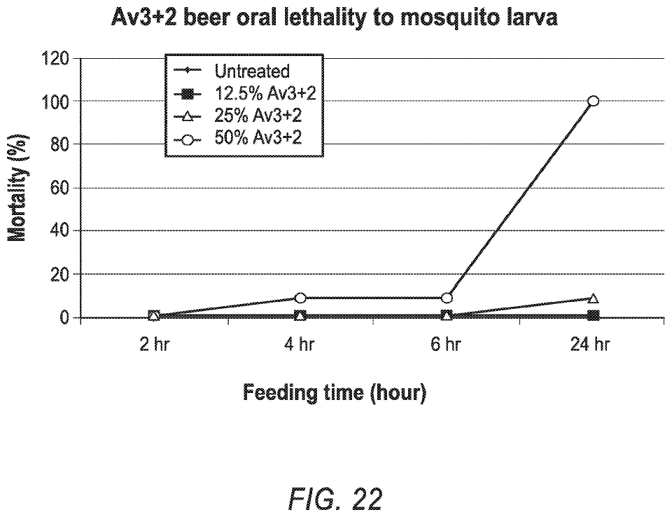

Av3+2 fermentation beer was found to be toxic to mosquito larva when consumed and/or during oral contact. A 2 L Fermentation was performed using the Av3+2 expression strain, pLB102-YCT-18, and the resulting fermentation beer was filtered to remove the yeast cells. The cell-free Av3+2 beer was mixed with water to provide the volume ratio of 50% (115 ppm), 25% (57.5 ppm) and 12.5% (28.8 ppm), in which mosquito larva (Aedes aegypti) were reared: 12.5% Av3 beer had no effect on the mosquito larva in 24 hours; 25% Av3 beer caused 9% mortality at 24 hours; and 50% Av3+2 beer caused 9% mortality after 4 hours feeding, and 100% mortality at 24 hours. FIG. 22.

### Example 8

### Contact Insecticidal Activity Against Adult Mosquito Using Av3 Peptides and AVPs

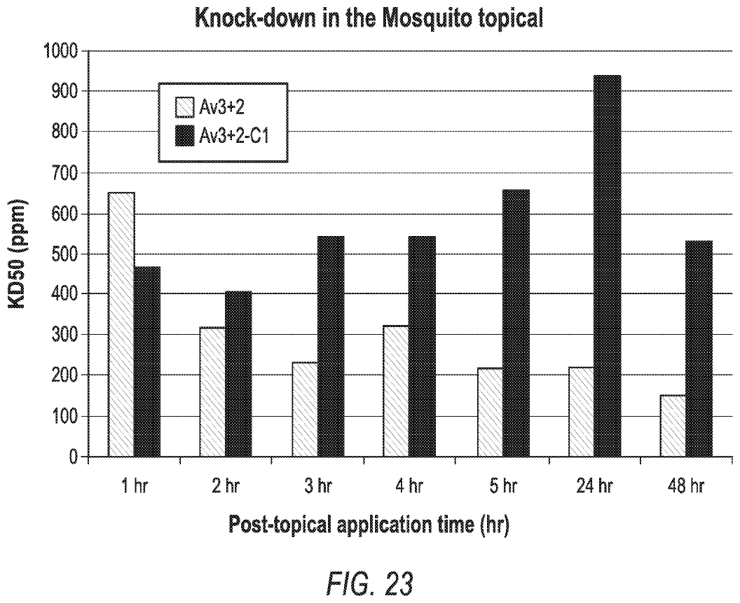

Av3+2 peptide kills adult mosquitos following topical application. The Av3+2 and Av3+2−C1 peptides were shown to fast paralyze and eventually kill adult mosquitoes following topical application. Both Av3+2 and Av3+-C1 caused knock-down in adult mosquitoes as early as 1-hour post-topical application. FIG. 23. Av3+2 knock-down effect reached its maximum at 3 hours, with not much recovery effects observed thereafter. At 3 hours the KD₅₀ was 235.71 ppm, a result similar to that of the first set (i.e. which was 229.9 ppm). FIG. 23. The Av3+2−C1 knock-down effect reached its maximum at 2 hours, but recovery was observed thereafter. At 3 hours, the KD₅₀ was 543.12 ppm, lower than that which was observed the first set (i.e., 952.69 ppm). At almost all the recorded times, Av3+2 had a lower KD₅₀ than Av3+2−C1. At 3 hours, KD₅₀ of Av3+2−C1 was almost two-fold higher than that of Av3+2. FIG. 23. These results indicate that the truncation at C-Val reduced the paralysis activity of Av3+2 peptide.

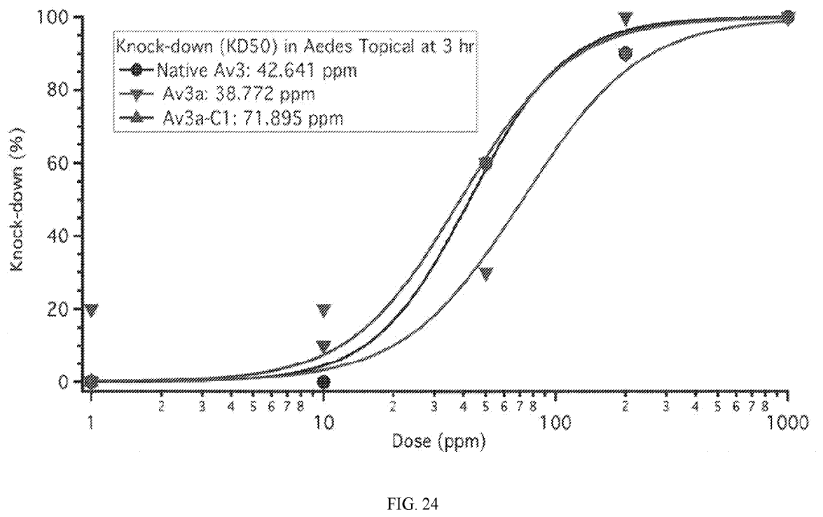

Native Av3, and the AVPs: R1K mutant Av3 (Av3a) and R1K+AC-Val double mutant Av3 (Av3a-C1, or Av3b) were evaluated in the adult mosquito topical bioassay. Native Av3 had KD₅₀ of 42.6 ppm at 3 hours post application. Av3a had a KD₅₀ of 38.8 ppm at 3 hours post application, the same as native Av3; these results indicated that R1K mutation did not cause activity reduction. FIG. 24. Av3a-C1 had KD₅₀ of 71.9 ppm at 3 hours post application, about twice as less active than native Av3, but much better than Av3+2 (200-300 ppm) and Av3+2−C1 (500-900 ppm). FIG. 24. A summary of the AVPs and their activities against adult mosquitos when applied topically can be seen in Table 5.

TABLE 5
Summary of the AVPs and their activities against adult
mosquitos when applied topically.

| Activity | Av3 | Av3 + 2 | Av3 + 2-C1 | Av3a | Av3a-C1 |
|---|---|---|---|---|---|
| Knock- | 19.3 | 230 | 952.7 | 179.3 | 86.9 |
| down | 9.97 | 235.7 | 543.1 | 38.8 | 86.1 |
| 50 at 3 hr | 58 | 347.7 | | | 71.9 |
| (ppm ) | 118.8 | 312.6 | | | |
| | 27.2 | | | | |
| | 42.6 | | | | |
| Average | 45.9783333 | 281.5 | 747.9 | 109.05 | 81.6333333 |
| SD | 39.5471569 | 58.0216626 | 289.630938 | 99.3485028 | 8.43879928 |

### Example 9

### Insecticidal Activities Synergy Between Av3 Peptides, AVPs, and Other Peptides or Insecticides (e.g., AaIT1, Bti, and Pymethrin)

AVPs exhibits synergy when used with Bti in a mosquito larva feeding assay. Av3+2 and Bti (Bacillus thuringiensis var. israelensis) synergistic effects were observed in the mosquito larva feeding bioassay. Av3+2 fermentation beer was used for the test, which contained both Av3+2 and Av3+2−C1, wherein the total estimated concentration of both was about 230 mg/L, i.e. 230 ppm. Doses were applied at concentrations of: 50%, 25% and 12.5% dilution. The Bti product used was Aquabac DF3000 from Arbico Organics, and its Al is Bti (Bacillus thuringiensis var. israelensis); with the applied dose at 50 and 100 ppb.

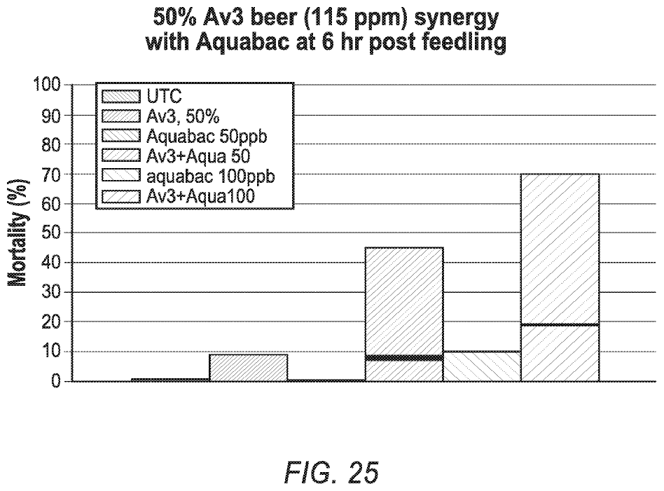

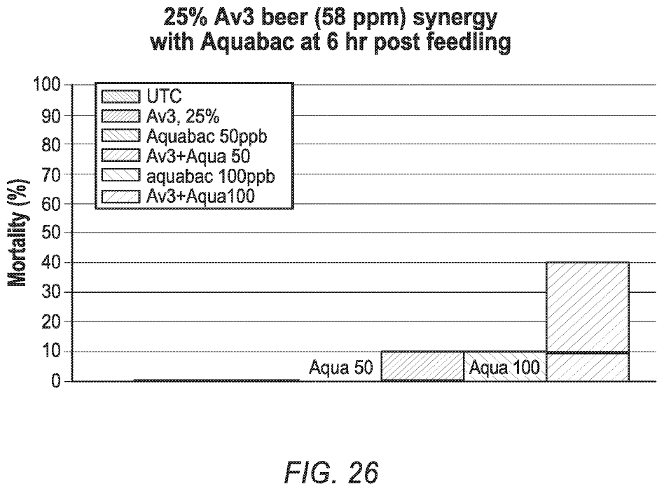

Av3+2 beer at 12.5%, 25% and 50% dilution mixture, with 50 ppb or 100 ppb Aquabac, resulted in a much higher mosquito larva mortality at 6 hours post feeding than individual components, and/or the mortality expected from additive effects, indicating a positive synergistic effect of pesticides and AVPs when used in the mosquito larva feeding bioassay. FIG. 25 and FIG. 26.

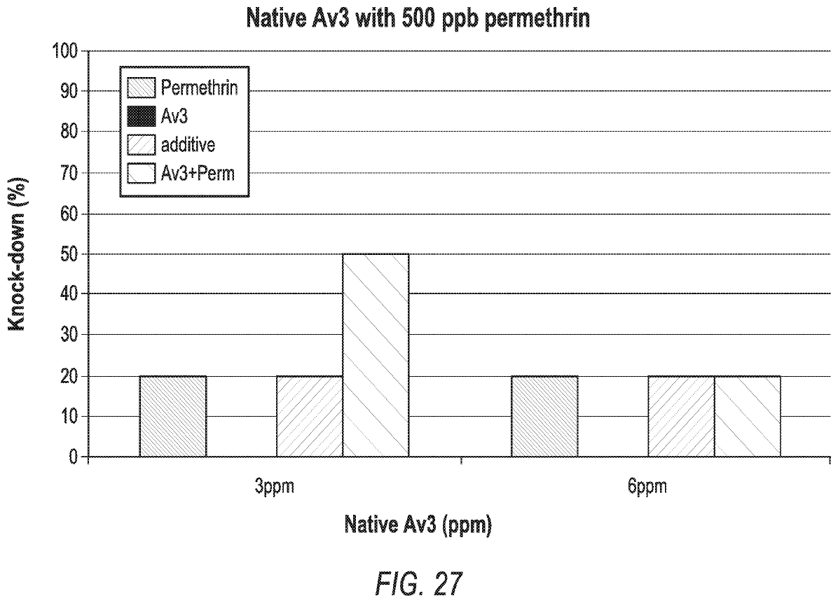

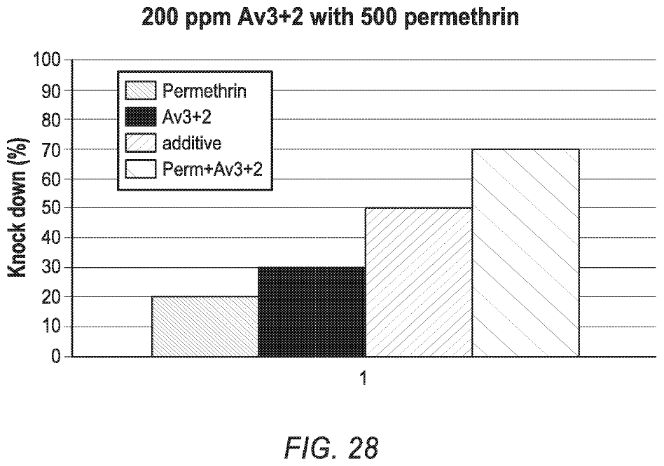

Av3 shows positive synergy when combined with permethrin in a mosquito bioassay. Av3+2 and native Av3 peptide synergy with permethrin was studied in the adult mosquito topical bioassay. An Av3+2 peptide at dose of 200 ppm showed some synergy with permethrin at the dose of 500 ppb. FIG. 27. Native Av3 peptide at a dose of 3 ppm, showed some synergy with permethrin at a dose of 500 ppb, but not at a dose of 6 ppm of Av3. FIG. 28.

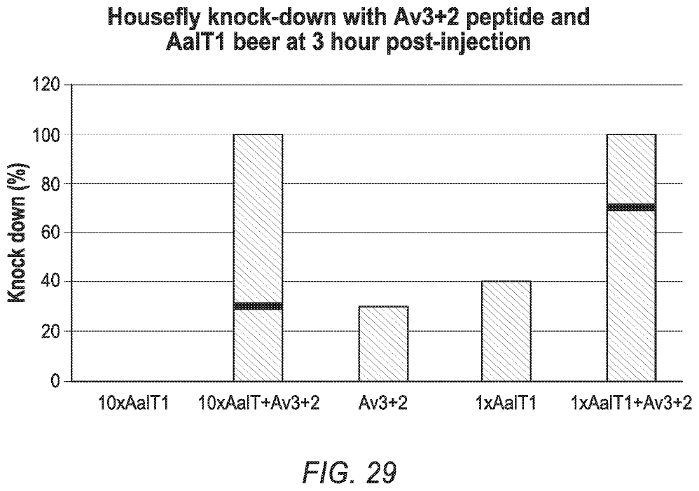

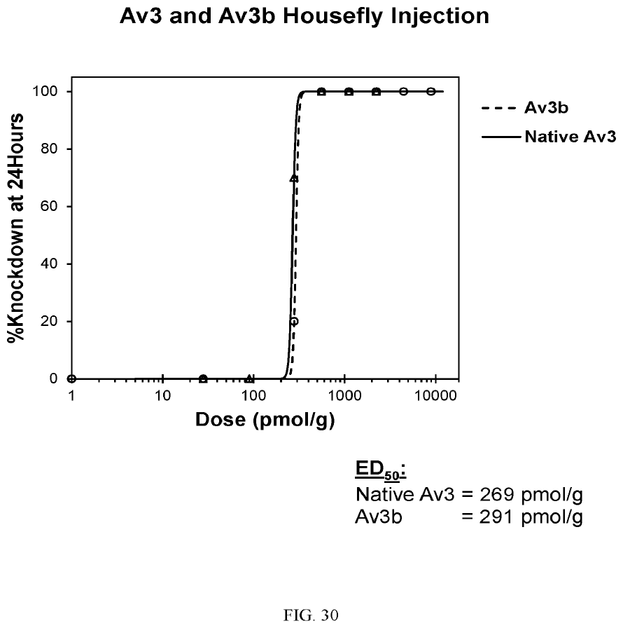

Av3+2 demonstrates positive synergy when combined with AaIT1. Av3 is a peptide that targets the insect sodium channel insecticide receptor site 3, which inhibits the inactivation of the channel. Site 4 toxins promote the insect sodium channel to open by lowering the activation reaction energy barrier. Site 3 and site 4 toxins both promote the insect sodium channel to open, albeit in different ways. AalT1 is a sodium channel site 4 toxin from North African desert scorpion (Androctonus australis). The fermentation beer from a AalT1 expression K. lactis strain showed synergistic instead of additive paralysis effect in the housefly injection bioassay with Av3+2 peptide at 500 pmol/g dose. FIG. 29. FIG. 30 depicts the 24 hour housefly knock-down assay using Av3 native polypeptide versus Av3b polypeptide and their resultant ED₅₀ concentrations.

### Example 10

### Defining and Characterizing the Av3 Pharmacophore, and Synthesizing Polypeptides Thereto

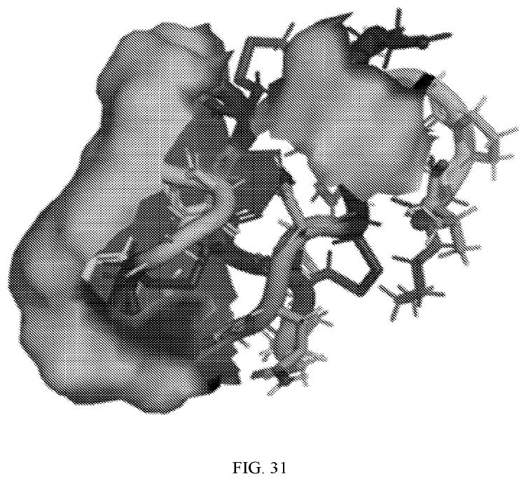

AVP has a hydrophobic nicotinic acetylcholine (NaCh) binding surface, which is involves the residues at positions P5, Y7, W8, P12 and W13, i.e., for an AVP. FIG. 31. The pharmacophore differs from other NaCh site 3 toxins, which involves charged amino acid for binding. Based on the identity and configuration of the pharmacophore in AVPs, a strategy was employed wherein alternate polypeptides (i.e., relative to WT Av3 or mutant Av3 polypeptides) were developed. The following general mutational strategy was used to discover proteins that utilized the key motif: (1) retention of the AVP disulfide bonds at positions 3-17, 4-11, 6-22 (i.e. 1-5, 2-4 & 3-6); (2) variation of the N-Lys or Arg; an Alanine scan, i.e., mutation of all amino acids to Alanine other than positions in the aforementioned (1) and/or (2), along with the key motif. The resulting design was expected to change the peptide confirmation of beta or gamma-turns owing to the mutation of Glycine. Finally, a mutation screen was performed where, the pharmacophore, the disulfide bonds, and the N-terminal K and/or R remained unchanged, the key Glycine turns remained unchanged (i.e., G9, G10 & G14 of SEQ ID NO:4), and mutated all variable element positions to Alanine; thus, this design attempted to minimize conformational change by keeping the key turns in the protein structure.

Several polypeptides were generated following the foregoing method, e.g., the following: Core-5 with the amino acid sequence “KACCPCYWGGCPWGAACYPAGCAAAK” (SEQ ID NO:30); Core-4 with the amino acid sequence “KACCPCYWAACPWAAACYAAACAAAK” (SEQ ID NO:31); Core-3, with the amino acid sequence “KPYWPWYK” (SEQ ID NO:32); Core-2, with the amino acid sequence “KPYWPWYKV” (SEQ ID NO:33); and Core-1, with the amino acid sequence “PYWPWY” (SEQ ID NO:34). The designed Av3 variant peptides were chemically synthesized by GenScript® (Piscataway, NJ). The synthetic peptides were resuspended into water (Av3b, Core 2, Core 3, Core 4 and Core 5) or DMSO (Core 1). Reverse phase HPLC (rpHPLC) and LC/MS was then performed to evaluate the peptides.

### Example 11

### Evaluation of Synthetic Polypeptides

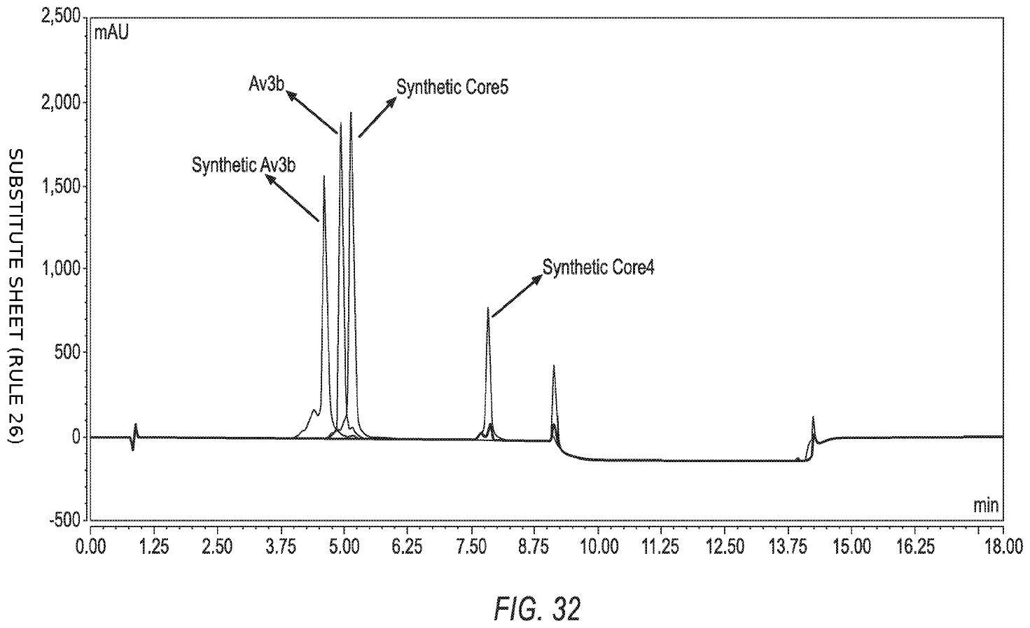

The synthetic polypeptides containing the key motif pharmocophore were validated using Agilent HPLC. rpHPLC was performed in Agilent 1100 HPLC instrument with water and Acetonitrile with 0.1% Trifluoroacitic acid (TFA) as mobile phases. And 23-37% acetonitrile gradient was used to elute the synthetic peptides in a 11 minute protocol. GenScript Synthesized Av3b (i.e., SEQ ID NO:4), AVP-Core-4 and AVP-Core-5 were. There was peak shift of synthetic Av3b comparing with the Av3b purified from the strain designated for potential use in forthcoming projects, indicating the lack of a disulfide bond formation in the synthetic Av3b. FIG. 32.

Liquid chromatography/mass spectrometry (LC/MS) was also used to validate the synthetic core polypeptides. The LC/MS system consisted of a Waters/Micromass LCT electrospray time-of-flight mass spectrometer on-line with an Agilent 1100 HPLC system via an electrospray ionization source. Ten to 30 uL μL of sample was injected onto a Waters C-18 X-Bridge Column (4.6 mm ID×50 mm, colume V=0.83 mL) at a flow rate of 1 mL/min. Reverse-phase separation was achieved over 15 minutes using a linear gradient of 99% mobile phase A (water with 0.1% formic acid) to 95% mobile phase B (100% acetonitrile with 0.1% formic acid) over 6 minutes, 95% B at 11 minutes, and 1% B at 11.2 minutes for a total run time of 18 minutes. Column outlet was flow-split into an Agilent 1100 diode-array UV detector and the LCT at a ratio of approximately 25:1 respectively. The LCT mass spectrometer collected positive ion data over a 100-2500 m/z mass range. Masslynx 4.1 software was used for instrument control and data acquisition. Masslynx MaxEnt 1 algorithm was used for deconvolution of multiply charged ions to a calculated M+H average mass value. Synthetic Av3b, Core 4 and Core 5 were validated in Walk-up LC/MS in LauchMI Lab. Table. 6 and Table. 7. LC/MS confirmed that none of synthetic Av3b, core 4 or core 5 had any disulfide-bond formation.

TABLE 6
Liquid chromatography/mass spectrometry readout of yeast-
expressed Av3b and synthetic polypeptides of different configurations.

| Sample | | Sample | | peptide | |
|---|---|---|---|---|---|
| No. | Sample | description | Preparation | Info | Results |
| 1 | dH2O | filtered water | blank control, | (—) | |
| | | | load 10 μL | control | |
| 2 | Av3b | HPLC | Diluted to 0.5 | M.W. | detected |
| | | purified | μg/μL: 10 μL | 2805.2 | M.W. |
| | | Av3b, from | stock + 190 μL | | 2805.5 |
| | | Av3b box | dH2O Load | | |
| | | 20 μL aliquot, | | | |
| | | 50 μg/μL | | | |
| 3 | Av3b | Synthetic, | Diluted to 0.5 | M.W. | detected |
| | (synthetic) | ~86% pure, | μg/μL: 10 μL | 2805.2 | M.W. |
| | | 10 μL from | stock + 190 μL | | 2810.9 |
| | | stock #6-1 | dH2O Load | | |
| 4 | Av3b | Synthetic, | Diluted to 0.5 | M.W. | detected |
| | Core 4 | ~86% pure, | μg/μL: 10 μL | 2639.15 | M.W. |
| | (synthetic) | 10 μL from | stock + 190 μL | | 2639.95 |
| | | stock #6-10 | dH2O Load | | |
| 5 | Av3b | Synthetic, | Diluted to 0.5 | M.W. | detected |
| | Core 5 | ~86% pure, | μg/μL: 10 μL | 2609.08 | M.W. |
| | (synthetic) | 10 μL from | stock + 190 μL | | 2639.95 |
| | | stock #6-28 | dH2O Load | | |

TABLE 7
Liquid chromatography/mass spectrometry showing mass-to-charge (m/z) ratios and the presence of disulfide bonds
for AVP-Core synthetic polypeptides.

| Possible | Theoretical | SEQ | Sequence/ | Possible charge | | | | | | |
|---|---|---|---|---|---|---|---|---|---|---|
| peptides | M.W. | ID NO | formula | 3 | 4 | 5 | 6 | 7 | 8 | 9 |
| Av3b with | 2805.2 | 4 | KSCCPCYWGGCPW | 936.066667 | 702.3 | 562.04 | 468.533333 | 401.742857 | 351.65 | 312.688889 |
| -s-s- | | | GQNCYPEGCSGPK | | | | | | | |
| Av3b no | 2811.2 | 4 | KSCCPCYWGGCPW | 938.066667 | 703.8 | 563.24 | 469.533333 | 402.6 | 352.4 | 313.355556 |
| -s-s- | | | GQNCYPEGCSGPK | | | | | | | |
| Core4 with | 2633.15 | 31 | KACCPCYWAACPW | 878.716667 | 659.2875 | 527.63 | 439.858333 | 377.164286 | 330.14375 | 293.572222 |
| -s-s- | | | AAACYAAACAAAK | | | | | | | |
| Core4 no | 2639.15 | 31 | KACCPCYWAACPW | 880.716667 | 660.7875 | 528.83 | 440.858333 | 378.021429 | 330.89375 | 294.238889 |
| -s-s- | | | AAACYAAACAAAK | | | | | | | |
| Core5 with | 2603.08 | 30 | KACCPCYWGGCPW | 868.693333 | 651.77 | 521.616 | 434.846667 | 371.868517 | 326.385 | 290.231111 |
| -s-s- | | | GAACYPAGCAAAK | | | | | | | |
| Core5 no | 2609.08 | 30 | KACCPCYWGGCPW | 870.693333 | 653.27 | 522.816 | 435.846667 | 373.725714 | 327.135 | 290.897778 |
| -s-s- | | | GAACYPAGCAAAK | | | | | | | |

### Example 12

### Housefly Injection Assay Using AVP-Core Synthetic Polypeptides

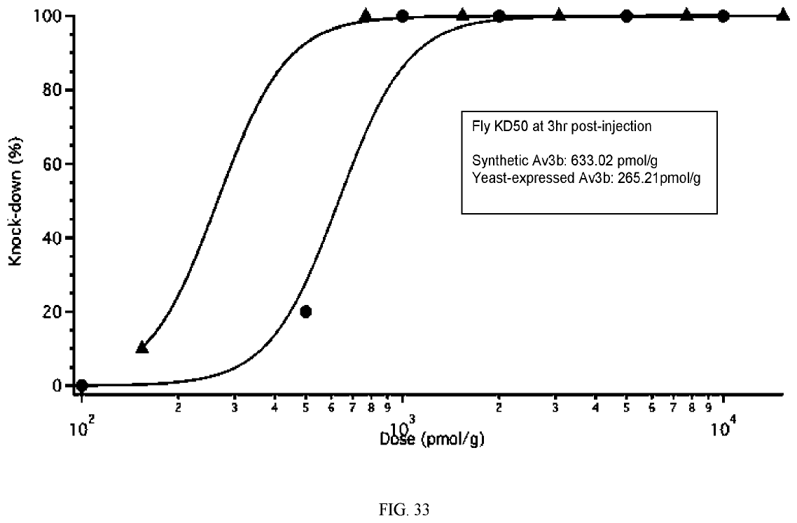

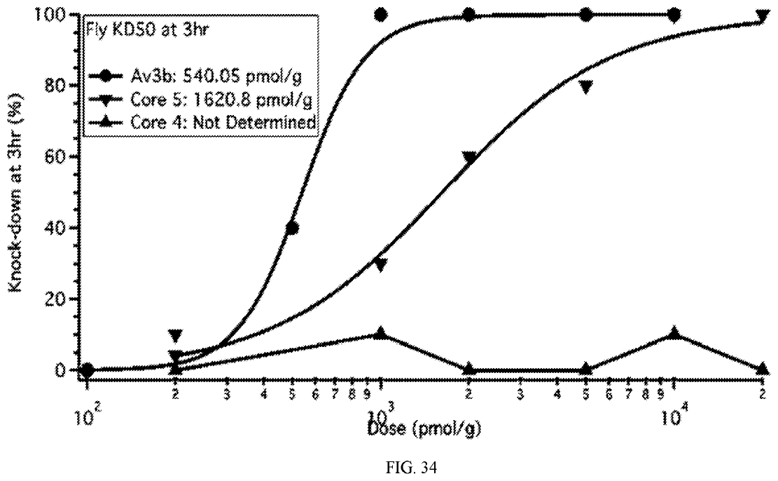

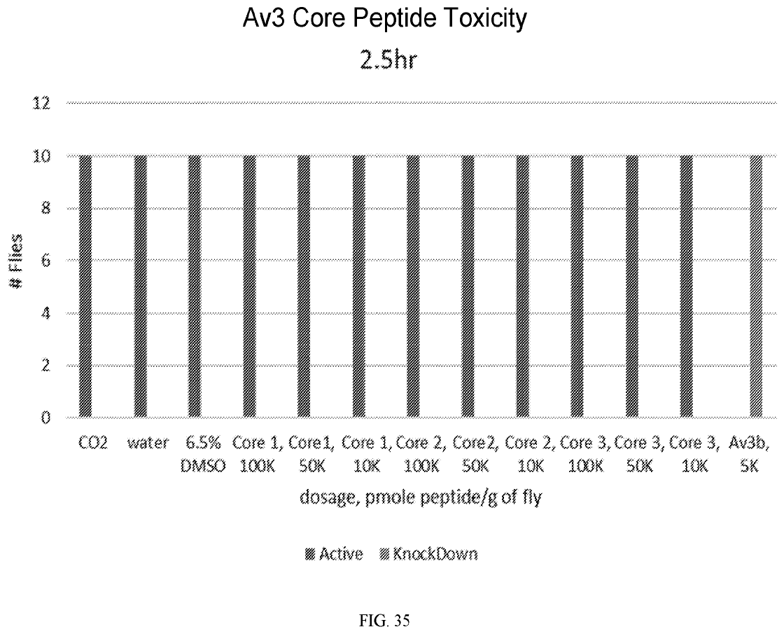

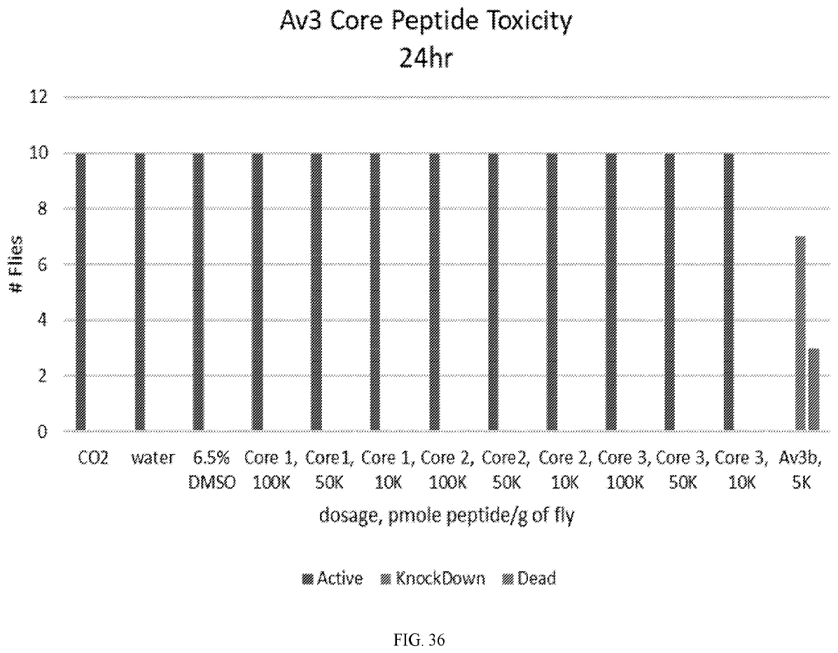

A Housefly injection assay was performed to compare the activity of Av3b with GenScript Synthetic Av3b. FIG. 33. Adult houseflies were immobilized in CO2 for 10 minutes and then transferred to a CO2 pan to keep them immobilized. Flies with weight between 12-20 mg were picked for injection. The synthetic peptides were diluted in water to proper doses for injection. 0.5 μL peptide solution was injected into housefly at the dorsal thorax using a hand-microapplicator with 1 cc all-glass syringe with 30 gouge straight needle. The injected flies were then transferred to a 2 oz transparent portion container with a wet #4 filter paper. Fly score was accessed at 2 hour, 3 hour, 4 hour and 24 hour post-injection. The Housefly injection indicated that yeast-expressed Av3b was 2-3 times more active than synthetic Av3b, likely due to the missing disulfide-bonds formation in the synthetic. Core-5 and Core-4 synthetic polypeptides were also evaluated in the housefly injection assay. FIG. 34. Consistent with previous results, synthetic linear Av3b showed activities in fly injection assay, i.e., quick knock-down. Core 4 peptide, which has Alanine mutations at the variable element positions (i.e., residues except the pharmacophore and cysteines residue locations), had dramatically reduced activities in fly injection assay. Core-5 polypeptide, which has Alanine mutations at variable element positions, not including the pharmacophore, cysteines or beta turn amino acids, had its activity reduced around 3-fold. FIG. 35. Core-1; Core-2; and Core-3 synthetic polypeptides did not appear to have an effect on housefly mortality when using a dose of 10, 50, and 100 nmol/g for 24 hours. FIG. 36. Accordingly, synthetic linear Av3b was still active against houseflies, but potency was reduced 2-3 time compared to yeast-expressed Av3b. Mutant Av3b polypeptide with a retained pharmacophore and cysteines, but missing all turn structures, lost a significant portion of its insecticidal activity against houseflies.

TABLE 8
Results of a housefly injection assay using yeast-expressed
Av3b and synthetic Av3b.

| | Yeast-expressed Av3b | | |
|---|---|---|---|
| | AAA Calibrate stock | non-AAA stock | Synthetic |
| | at 32.5 mg/mL | at 50 mg/mL | Av3b |
| KD50 at 3 hr | 172.22 pmol/g | 265.21 pmol/g | 633.02 pmol/g |

TABLE 9
Comparison of KD50 for synthetic Av3 polypeptides
and AVP-Core synthetic polypeptides.

| | Synthetic Av3b | Core 4 | Core 5 |
|---|---|---|---|
| KD50 at 3hr | 540 pmol/g | N/D | 1621 pmol/g |

1. An Av3 variant polypeptide having insecticidal activity against one or more insect species, wherein the Av3 variant polypeptide comprises at least one of the following mutations:
(a) an N-terminus mutation replacing arginine with lysine (R1K) relative to SEQ ID NO:1; and
(b) a deletion of the C-terminus valine amino acid relative to SEQ ID NO:1.

2. The Av3 variant polypeptide of claim 1, wherein the Av3 variant polypeptide comprises an N-terminus mutation replacing arginine with lysine (R1K), and a deletion of the C-terminus valine amino acid, relative to SEQ ID NO:1.

3. The Av3 variant polypeptide of claim 1, wherein the Av3 variant polypeptide further comprises a homopolymer or heteropolymer of two or more Av3 variant polypeptides, wherein the amino acid sequence of each Av3 variant polypeptide is the same or different.

4. The Av3 variant polypeptide of claim 1, wherein the Av3 variant polypeptide is a fused protein comprising two or more Av3 variant polypeptides separated by a cleavable or non-cleavable linker, and wherein the amino acid sequence of each Av3 variant polypeptide may be the same or different.

5. The Av3 variant polypeptide of claim 4, wherein the linker is cleavable inside at least one of: (i) the gut or hemolymph of an insect; and (ii) cleavable inside the gut of a mammal.

6. (canceled)

7. A plant, plant tissue, plant cell, or plant seed comprising: an Av3 variant polypeptide of claim 1, or a polynucleotide encoding the same; wherein the Av3 variant polypeptide comprises at least one of the following mutations:
(a) an N-terminus mutation replacing arginine with lysine (R1K) relative to SEQ ID NO:1;
(b) a deletion of the C-terminus valine amino acid relative to SEQ ID NO:1.

8. The plant, plant tissue, plant cell or seed of claim 7, wherein the Av3 variant polypeptide, or polynucleotide encoding the same, comprises an N-terminus mutation replacing arginine with lysine (R1K), and a deletion of the C-terminus valine amino acid, relative to SEQ ID NO:1.

9. A polynucleotide operable to encode an Av3 variant polypeptide, or a complementary polynucleotide sequence thereof, wherein the Av3 variant polypeptide comprises at least one of the following mutations:
(a) an N-terminus mutation replacing arginine with lysine (R1K) relative to SEQ ID NO:1;
(b) a deletion of the C-terminus valine amino acid relative to SEQ ID NO:1.

10. The polynucleotide of claim 9, or the complementary polynucleotide sequence thereof, wherein the Av3 variant polypeptide comprises an N-terminus mutation replacing arginine with lysine (R1K), and a deletion of the C-terminus valine amino acid, relative to SEQ ID NO:1.

11.-30. (canceled)

31. An Av3 variant polypeptide comprising an amino acid sequence that is at least 90%, 95%, 96%, 97%, 97%, 98%, 99%, or 100% identical to an amino acid sequence set forth in any one of SEQ ID NOs: 2-4, and wherein the Av3 variant polypeptide does not consist of the amino acid sequence of SEQ ID NO: 1.

32. An Av3 variant polypeptide consisting of an amino acid sequence of any on one of SEQ ID NOs: 2-4.

33. A composition comprising an Av3 variant polypeptide of claim 31, and an excipient.

34. A method of producing an AVP, the method comprising:
a. preparing a vector comprising a first expression cassette comprising a polynucleotide operable to express a AVP having at least one mutation selected from: an N-terminal mutation and a C-terminal mutation relative to the wild-type sequence of Av3 as set forth in SEQ ID NO:1;
b. introducing the vector into a yeast strain;
c. growing the yeast strain in a growth medium under conditions operable to enable expression of the AVP and secretion into the growth medium, and
d. isolating the expressed AVP from the growth medium.

35. The method of claim 34, wherein the N-terminal mutation is an amino acid substitution of R1K relative to SEQ ID NO:1.

36. The method of claim 34, wherein the C-terminal mutation is an amino acid deletion of the C-terminal valine relative to SEQ ID NO:1.

37. The method of claim 34, wherein the polynucleotide encodes AVP having an N-terminal mutation comprising an amino acid substitution of R1K relative to SEQ ID NO:1, and a C-terminal mutation comprising an amino acid deletion of the C-terminal valine relative to SEQ ID NO:1.

38. The method of claim 34, wherein the vector is a plasmid comprising an alpha-MF signal.

39. The method of claim 38, wherein the plasmid further comprises a Kex 2 cleavage site.

40. The method of claim 34, wherein the vector is transformed into a yeast strain.

41. The method of claim 40, wherein the yeast strain is selected from any species of the genuses Saccharomyces, Pichia, Kluyveromyces, Hansenula, Yarrowia or Schizosaccharomyces.

42. The method of claim 41, wherein the yeast strain is Kluyveromyces lactis.


SEQUENCE LISTING (38 sequences)

SEQ ID NO: 1 - protein, 27 aa, Anemonia viridis
RSCCPCYWGGCPWGQNCYPEGCSGPKV

SEQ ID NO: 2 - protein, 27 aa, synthetic construct
  REGION 1..27: note = Description of Artificial Sequence: Synthetic polypeptide
  REGION 1..27: note = N-terminal amino acid substitution of R1K relative to SEQ ID NO: 1
KSCCPCYWGGCPWGQNCYPEGCSGPKV

SEQ ID NO: 3 - protein, 26 aa, synthetic construct
  REGION 1..26: note = Description of Artificial Sequence: Synthetic polypeptide
  REGION 1..26: note = C-terminal amino acid deletion relative to SEQ ID NO: 1
RSCCPCYWGGCPWGQNCYPEGCSGPK

SEQ ID NO: 4 - protein, 26 aa, synthetic construct
  REGION 1..26: note = Description of Artificial Sequence: Synthetic polypeptide
  REGION 1..26: note = N-terminal amino acid substitution of R1K and C-terminal amino acid deletion
KSCCPCYWGGCPWGQNCYPEGCSGPK

SEQ ID NO: 5 - protein, 24 aa, synthetic construct
  REGION 1..24: note = Description of Artificial Sequence: Synthetic polypeptide
  REGION 1..24: note = Barley Alpha-Amylase Signal peptide (BAAS)
MANKHLSLSLFLVLLGLSASLASG

SEQ ID NO: 6 - protein, 4 aa, synthetic construct
  REGION 1..4: note = Description of Artificial Sequence: Synthetic polypeptide
  REGION 1..4: note = Cleavable type of intervening linker
IGER

SEQ ID NO: 7 - protein, 5 aa, synthetic construct
  REGION 1..5: note = Description of Artificial Sequence: Synthetic polypeptide
  REGION 1..5: note = Intervening linker peptide
EEKKN

SEQ ID NO: 8 - protein, 8 aa, synthetic construct
  REGION 1..8: note = Description of Artificial Sequence: Synthetic polypeptide
  REGION 1..8: note = Uncleavable intervening linker peptide
ETMFKHGL

SEQ ID NO: 9 - protein, 238 aa, synthetic construct
  REGION 1..238: note = Description of Artificial Sequence: Synthetic polypeptide
  REGION 1..238: note = Green-Fluorescent Protein (GFP)
ASKGEELFTGVVPILVELDGDVNGHKFSVSGEGEGDATYGKLTLKFICTTGKLPVPWPTLVTTFSYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITHGMDELYK

SEQ ID NO: 10 - protein, 157 aa, synthetic construct
  REGION 1..157: note = Description of Artificial Sequence: Synthetic polypeptide
  REGION 1..157: note = Translational stabilizing protein
MAKASLLILATIFLGVITPSCLSENILYSGETLPTGGFLSSGSFVFIMQEDCNLVLYNVDKPIWATNTGGLSSDCSLSMQNDGNLVVFTPSNKPIWASNTDGQNGNYVCILQKDRNVVIYGTNRWATGTYTGAVGIPESPPSEKYPSAGKIKLVTAK

SEQ ID NO: 11 - protein, 224 aa, synthetic construct
  REGION 1..224: note = Description of Artificial Sequence: Synthetic polypeptide
  REGION 1..224: note = Translational stabilizing protein
KFDIKNQCGYTVWAAGLPGGGKRLDQGQTWTVNLAAGTASARFWGRTGCTFDASGKGSCQTGDCGGQLSCTVSGAVPATLAEYTQSDQDYYDVSLVDGFNIPLAINPTNAQCTAPACKADINAVCPSELKVDGGCNSACNVFKTDQYCCRNAYVDNCPATNYSKIFKNQCPQAYSYAKDDTATFACASGTDYSIVFCMARVSELAFLLAATLAISLHMQEAGVV

SEQ ID NO: 12 - protein, 27 aa, synthetic construct
  REGION 1..27: note = Description of Artificial Sequence: Synthetic polypeptide
  REGION 1..27: note = ESRP
EMGKMASLFASLLVVLVSLSLASESSA

SEQ ID NO: 13 - protein, 26 aa, synthetic construct
  REGION 1..26: note = Description of Artificial Sequence: Synthetic polypeptide
  REGION 1..26: note = ESRP
MGKMASLFATFLVVLVSLSLASESSA

SEQ ID NO: 14 - DNA, 78 bp, synthetic construct
  misc_feature 1..78: note = Description of Artificial Sequence: Synthetic polynucleotide
  misc_feature 1..78: note = DNA sequence encoding tobacco extensin signal peptide variant 1
  source 1..78: mol_type = other DNA
atgggtaagatggcttctctgtttgcttctctgctggttgttctggtttctctgtctctggcttctgaatcttctgct

SEQ ID NO: 15 - DNA, 78 bp, synthetic construct
  misc_feature 1..78: note = Description of Artificial Sequence: Synthetic polynucleotide
  misc_feature 1..78: note = DNA sequence encoding tobacco extensin signal peptide variant 2
  source 1..78: mol_type = other DNA
atgggtaagatggcttctctgtttgctacttttctggttgttctggtttctctgtctctggcttctgaatcttctgct

SEQ ID NO: 16 - protein, 224 aa, synthetic construct
  REGION 1..224: note = Description of Artificial Sequence: Synthetic polypeptide
  REGION 1..224: note = Translational stabilizing protein
MARVSELAFLLAATLAISLHMQEAGVVKFDIKNQCGYTVWAAGLPGGGKRLDQGQTWTVNLAAGTASARFWGRTGCTFDASGKGSCQTGDCGGQLSCTVSGAVPATLAEYTQSDQDYYDVSLVDGFNIPLAINPTNAQCTAPACKADINAVCPSELKVDGGCNSACNVFKTDQYCCRNAYVDNCPATNYSKIFKNQCPQAYSYAKDDTATFACASGTDYSIVFC

SEQ ID NO: 17 - protein, 105 aa, synthetic construct
  REGION 1..105: note = Description of Artificial Sequence: Synthetic polypeptide
  REGION 1..105: note = Translational stabilizing protein
DNILYSGETLSTGEFLNYGSFVFIMQEDCNLVLYDVDKPIWATNTGGLSRSCFLSMQTDGNLVVYNPSNKPIWASNTGGQNGNYVCILQKDRNVVIYGTDRWATG

SEQ ID NO: 18 - protein, 29 aa, synthetic construct
  REGION 1..29: note = Description of Artificial Sequence: Synthetic polypeptide
  REGION 1..29: note = Av3’peptide sequence ("Peak 2")
GSRSCCPCYWGGCPWGQNCYPEGCSGPKV

SEQ ID NO: 19 - protein, 28 aa, synthetic construct
  REGION 1..28: note = Description of Artificial Sequence: Synthetic polypeptide
  REGION 1..28: note = Av3’peptide with C-terminal valine cleaved ("Peak 1")
GSRSCCPCYWGGCPWGQNCYPEGCSGPK

SEQ ID NO: 20 - protein, 29 aa, synthetic construct
  REGION 1..29: note = Description of Artificial Sequence: Synthetic polypeptide
  REGION 1..29: note = Mutation strategy strain
GSRSCCPCYWGGCPWGQNCYPEGCSGPAV

SEQ ID NO: 21 - protein, 29 aa, synthetic construct
  REGION 1..29: note = Description of Artificial Sequence: Synthetic polypeptide
  REGION 1..29: note = Mutation strategy strain
GSRSCCPCYWGGCPWGQNCYPEGCSGAAV

SEQ ID NO: 22 - protein, 29 aa, synthetic construct
  REGION 1..29: note = Description of Artificial Sequence: Synthetic polypeptide
  REGION 1..29: note = Mutation strategy strain
GSRSCCPCYWGGCPWGQNCYPEGCSGPHV

SEQ ID NO: 23 - protein, 29 aa, synthetic construct
  REGION 1..29: note = Description of Artificial Sequence: Synthetic polypeptide
  REGION 1..29: note = Mutation strategy strain
GSRSCCPCYWGGCPWGQNCYPEGCSGAHV

SEQ ID NO: 24 - protein, 29 aa, synthetic construct
  REGION 1..29: note = Description of Artificial Sequence: Synthetic polypeptide
  REGION 1..29: note = Mutation strategy strain
GSRSCCPCYWGGCPWGQNCYPEGCSGPDV

SEQ ID NO: 25 - protein, 28 aa, synthetic construct
  REGION 1..28: note = Description of Artificial Sequence: Synthetic polypeptide
  REGION 1..28: note = Mutation strategy strain
GSRSCCPCYWGGCPWGQNCYPEGCSGPK

SEQ ID NO: 26 - protein, 29 aa, synthetic construct
  REGION 1..29: note = Description of Artificial Sequence: Synthetic polypeptide
  REGION 1..29: note = Mutation strategy strain
GSRSCCPCYWGGCPWGQNCYPEGCSGPKT

SEQ ID NO: 27 - protein, 29 aa, synthetic construct
  REGION 1..29: note = Description of Artificial Sequence: Synthetic polypeptide
  REGION 1..29: note = Mutation strategy strain
GSRSCCPCYWGGCPWGQNCYPEGCSGPKA

SEQ ID NO: 28 - protein, 29 aa, synthetic construct
  REGION 1..29: note = Description of Artificial Sequence: Synthetic polypeptide
  REGION 1..29: note = Mutation strategy strain
GSRSCCPCYWGGCPWGQNCYPEGCSGPKP

SEQ ID NO: 29 - protein, 29 aa, synthetic construct
  REGION 1..29: note = Description of Artificial Sequence: Synthetic polypeptide
  REGION 1..29: note = Mutation strategy strain
GSRSCCPCYWGGCPWGQNCYPEGCSGPQV

SEQ ID NO: 30 - protein, 26 aa, synthetic construct
  REGION 1..26: note = Description of Artificial Sequence: Synthetic polypeptide
  REGION 1..26: note = Pharmacophore mutant
KACCPCYWGGCPWGAACYPAGCAAAK

SEQ ID NO: 31 - protein, 26 aa, synthetic construct
  REGION 1..26: note = Description of Artificial Sequence: Synthetic polypeptide
  REGION 1..26: note = Pharmacophore mutant
KACCPCYWAACPWAAACYAAACAAAK

SEQ ID NO: 32 - protein, 8 aa, synthetic construct
  REGION 1..8: note = Description of Artificial Sequence: Synthetic polypeptide
  REGION 1..8: note = Pharmacophore mutant
KPYWPWYK

SEQ ID NO: 33 - protein, 9 aa, synthetic construct
  REGION 1..9: note = Description of Artificial Sequence: Synthetic polypeptide
  REGION 1..9: note = Pharmacophore mutant
KPYWPWYKV

SEQ ID NO: 34 - protein, 6 aa, synthetic construct
  REGION 1..6: note = Description of Artificial Sequence: Synthetic polypeptide
  REGION 1..6: note = Pharmacophore mutant
PYWPWY

SEQ ID NO: 35 - protein, 27 aa, synthetic construct
  REGION 1..27: note = Description of Artificial Sequence: Synthetic polypeptide
  VARIANT 1: note = H, K, D, E, S, T, N, Q, C, G, P, A, V, I, L, M, F, Y, or W
  VARIANT 2: note = R, H, K, D, E, S, T, N, Q, C, G, P, A, V, I, L, M, F, Y, or W
  VARIANT 15: note = R, H, K, D, E, S, T, N, Q, C, G, P, A, V, I, L, M, F, Y, or W
  VARIANT 16: note = R, H, K, D, E, S, T, N, Q, C, G, P, A, V, I, L, M, F, Y, or W
  VARIANT 20: note = R, H, K, D, E, S, T, N, Q, C, G, P, A, V, I, L, M, F, Y, or W
  VARIANT 23: note = R, H, K, D, E, S, T, N, Q, C, G, P, A, V, I, L, M, F, Y, or W
  VARIANT 24: note = R, H, K, D, E, S, T, N, Q, C, G, P, A, V, I, L, M, F, Y, or W
  VARIANT 25: note = R, H, K, D, E, S, T, N, Q, C, G, P, A, V, I, L, M, F, Y, or W
  VARIANT 26: note = R, H, K, D, E, S, T, N, Q, C, G, P, A, V, I, L, M, F, Y, or W
  VARIANT 27: note = R, H, K, D, E, S, T, N, Q, C, G, P, A, V, I, L, M, F, Y, W or absent
XXCCPCYWGGCPWGXXCYPXGCXXXXX

SEQ ID NO: 36 - protein, 26 aa, synthetic construct
  REGION 1..26: note = Description of Artificial Sequence: Synthetic polypeptide
  VARIANT 1: note = R, H, K, D, E, S, T, N, Q, C, G, P, A, V, I, L, M, F, Y, or W
  VARIANT 2: note = R, H, K, D, E, S, T, N, Q, C, G, P, A, V, I, L, M, F, Y, or W
  VARIANT 15: note = R, H, K, D, E, S, T, N, Q, C, G, P, A, V, I, L, M, F, Y, or W
  VARIANT 16: note = R, H, K, D, E, S, T, N, Q, C, G, P, A, V, I, L, M, F, Y, or W
  VARIANT 20: note = R, H, K, D, E, S, T, N, Q, C, G, P, A, V, I, L, M, F, Y, or W
  VARIANT 23: note = R, H, K, D, E, S, T, N, Q, C, G, P, A, V, I, L, M, F, Y, or W
  VARIANT 24: note = R, H, K, D, E, S, T, N, Q, C, G, P, A, V, I, L, M, F, Y, or W
  VARIANT 25: note = R, H, K, D, E, S, T, N, Q, C, G, P, A, V, I, L, M, F, Y, or W
  VARIANT 26: note = R, H, K, D, E, S, T, N, Q, C, G, P, A, V, I, L, M, F, Y, or W
XXCCPCYWGGCPWGXXCYPXGCXXXX

SEQ ID NO: 37 - protein, 27 aa, synthetic construct
  REGION 1..27: note = Description of Artificial Sequence: Synthetic polypeptide
  VARIANT 1: note = H, K, D, E, S, T, N, Q, C, G, P, V, I, L, M, F, Y, or W
  VARIANT 2: note = R, H, K, D, E, S, T, N, Q, C, G, P, A, V, I, L, M, F, Y, or W
  VARIANT 15: note = R, H, K, D, E, S, T, N, Q, C, G, P, A, V, I, L, M, F, Y, or W
  VARIANT 16: note = R, H, K, D, E, S, T, N, Q, C, G, P, A, V, I, L, M, F, Y, or W
  VARIANT 20: note = R, H, K, D, E, S, T, N, Q, C, G, P, A, V, I, L, M, F, Y, or W
  VARIANT 23: note = R, H, K, D, E, S, T, N, Q, C, G, P, A, V, I, L, M, F, Y, or W
  VARIANT 24: note = R, H, K, D, E, S, T, N, Q, C, G, P, A, V, I, L, M, F, Y, or W
  VARIANT 25: note = R, H, K, D, E, S, T, N, Q, C, G, P, A, V, I, L, M, F, Y, or W
  VARIANT 26: note = R, H, K, D, E, S, T, N, Q, C, G, P, A, V, I, L, M, F, Y, or W
  VARIANT 27: note = R, H, K, D, E, S, T, N, Q, C, G, P, A, V, I, L, M, F, Y, W or absent
XXCCPCYWGGCPWGXXCYPXGCXXXXX

SEQ ID NO: 38 - protein, 26 aa, synthetic construct
  REGION 1..26: note = Description of Artificial Sequence: Synthetic polypeptide
  VARIANT 1: note = R, H, K, D, E, S, T, N, Q, C, G, P, V, I, L, M, F, Y, or W
  VARIANT 2: note = R, H, K, D, E, S, T, N, Q, C, G, P, A, V, I, L, M, F, Y, or W
  VARIANT 15: note = R, H, K, D, E, S, T, N, Q, C, G, P, A, V, I, L, M, F, Y, or W
  VARIANT 16: note = R, H, K, D, E, S, T, N, Q, C, G, P, A, V, I, L, M, F, Y, or W
  VARIANT 20: note = R, H, K, D, E, S, T, N, Q, C, G, P, A, V, I, L, M, F, Y, or W
  VARIANT 23: note = R, H, K, D, E, S, T, N, Q, C, G, P, A, V, I, L, M, F, Y, or W
  VARIANT 24: note = R, H, K, D, E, S, T, N, Q, C, G, P, A, V, I, L, M, F, Y, or W
  VARIANT 25: note = R, H, K, D, E, S, T, N, Q, C, G, P, A, V, I, L, M, F, Y, or W
  VARIANT 26: note = R, H, K, D, E, S, T, N, Q, C, G, P, A, V, I, L, M, F, Y, or W
XXCCPCYWGGCPWGXXCYPXGCXXXX